(19) United States
(12) Patent Application Publication
(10) Pub. No.: US 2024/0083904 A1
(43) Pub. Date: Mar. 14, 2024

(54) ANTAGONISTS OF THE ADENOSINE A2A RECEPTOR

(71) Applicant: AdoRx Therapeutics Limited, Edinburgh (GB)

(72) Inventors: Clive McCarthy, Edinburgh (GB); Benjamin Moulton, Alderley Edge (GB)

(21) Appl. No.: 18/266,428

(22) PCT Filed: Dec. 10, 2021
(86) PCT No.: PCT/GB21/53252

§ 371 Date: Jun. 9, 2023

(30) Foreign Application Priority Data
Dec. 11, 2020 (GB) 2019622.6

(51) Int. Cl. C07D 487/04; A61K 45/06

(52) U.S. Cl. CPC C07D 487/04; A61K 45/06

(57) ABSTRACT

The present invention relates to compounds of formula I shown below:

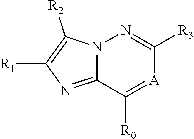

wherein R₀, R₁, R₂, R₃ and A are each as defined in the application. The present invention also relates to processes for the preparation of these compounds, to pharmaceutical compositions comprising them, and to their use in the treatment of diseases or conditions in which adenosine A2a receptor activity is implicated, such as, for example, cancer.

### INTRODUCTION

The present invention relates to certain compounds that function as antagonists of the adenosine A2a receptor. Additionally, some of the compounds are also antagonists of the A2b receptor. The present invention also relates to processes for the preparation of these compounds, to pharmaceutical compositions comprising them, and to their use in the treatment of diseases or conditions in which adenosine A2a receptor activity is implicated, such as, for example, cancer.

### BACKGROUND OF THE INVENTION

A number of immunosuppressive pathways are active in the tumour microenvironment which enable tumour cells to evade elimination by cytotoxic T cells and can diminish the clinical response of patients to immunotherapy with anti-checkpoint antibodies. The anti-PD-1 antibodies pembrolizumab and nivolumab and anti-PD-L1 antibodies durvalumab, avelumab and atezolizumab are approved for the treatment of number of solid tumours including non-small cell lung cancer, head and neck squamous cancer and urothelial cancer. However, only 20-30% of patients respond to checkpoint blockade and the side effects of such treatments are significant (Sukari et al, 2016). Consequently, other approaches to enhance the cytotoxic potential of the tumour microenvironment are actively being investigated. This includes agents that could be used as monotherapies or, more likely, used in combination with checkpoint inhibitors and cytotoxic agents to enhance their efficacy.

One approach that has attracted attention is to interfere with the production and/or action of adenosine in the tumour microenvironment (Vijayan et al, 2017). Adenosine has immunosuppressive properties and is present in the tumour microenvironment at high concentrations. Recent studies estimate the concentration of adenosine to be about 10 μM in human tumours compared to <1 μM in normal tissue (Houthuys et al 2017). Adenosine is formed at both intracellular and extracellular sites by two distinct pathways that involve two different substrates. Intracellular adenosine is derived from AMP and S-adenosyl homocysteine whilst the high extracellular adenosine concentrations observed during metabolic stress are associated with the release and degradation of precursor adenine nucleotides (ATP, ADP and AMP) by the concerted action of CD39 and CD73 (Vijayan et al, 2017).

CD39 and CD73 are upregulated in the tumour microenvironment in response to hypoxia. CD73 represents a putative patient stratification method for adenosine antagonists as its expression on tumour cells is also associated with poor overall prognosis in many different cancer types suggesting that adenosine production contributes to the undesirable immunosuppressive phenotype of the tumour microenvironment (Gao et al 2014; Loi et al, 2013;). CD73 expression by tumour-infiltrating immune cells is also important in promoting tumour immune suppression as CD73 negative Treg cells fail to suppress effector T cell functions (Deaglio et al, 2007; Reinhardt et al, (2017). Furthermore, patients resistant to anti-PD1 treatment have elevated levels of CD73 (Reinhardt et al, 2017).

Adenosine regulates cell function via occupancy of specific GPCRs on the cell surface of the P1 purinoceptor subtypes. The P1 receptor family is further subdivided into A1, A2a, A2b and A3.

A2 receptors are subdivided into A2a and A2b, based on high and low affinity for adenosine, respectively. A2a is expressed by lymphocytes and activation of A2a leads to suppression of cytokine production and other effector functions. Tumour growth is inhibited by genetic ablation of A2a in syngeneic mouse models and this effect has been demonstrated to be due to enhanced lymphocyte activation and cytotoxic function (Ohta et al, 2006; Waickman et al 2012; Beavis et al, 2013; Mittal et al, 2014; Cekic et al, 2014). A2a−/− mice show an increased response to inhibition of checkpoint pathways such as PD-1, with an improvement in both tumour free survival and overall survival. Adenosine-mediated A2a activation also limits the efficacy of anti-CTLA4 treatment (Iannone et al, 2014).

The effects of genetic deficiency of A2a in mouse models is mimicked by pharmacological blockade of A2a. A2a antagonists have been shown to enhance the cytotoxic CD8+ T cells and to enhance the ability of NK cells prevent metastasis of CD73-expressing tumours (Beavis et al, 2013). Importantly, A2a antagonists enhance the efficacy of anti-PD1 antibodies (Beavis et al, 2015).

These findings have prompted the development of selective A2a antagonists for use in cancer immunotherapy and clinical trials are ongoing with CPI-444, the first selective A2a antagonist to be evaluated in cancer, being used as both as a monotherapy and in combination with the anti-PDL1 antibody atezolizumab. The preliminary data indicated that the compound was well tolerated and showed early indications of reducing tumour size and enhancing CD8+T infiltration into tumour tissue.

However, there remains a need for second generation compounds that are potent adenosine A2a antagonists. In particular, there is a need for compounds that are potent and selective adenosine A2a antagonists and, in some cases, potent and selective adenosine A2a and A2b antagonists. There is also a need for compounds that are potent adenosine A2a antagonists or adenosine A2a and A2b antagonists that retain activity in the presence of the high concentrations of adenosine that are present in the tumour microenvironment.

### SUMMARY OF THE INVENTION

According to a first aspect of the present invention, there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of antagonising adenosine A2a receptors (and in some cases A2b receptors) in vitro or in vivo, said method comprising contacting a cell with an effective amount of a compound or a pharmaceutically acceptable salt, hydrate or solvate thereof as defined herein.

According to a further aspect of the present invention, there is provided a method of selectively antagonising adenosine A2a receptors (and in some cases A2b receptors) in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder associated with adenosine A2a receptor activity in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an adenosine A2a antagonist. In an embodiment, the compounds of the invention are selective adenosine A2a antagonists. In an alternative embodiment, certain compounds of the invention are selective adenosine A2a and adenosine A2b antagonists.

According to a further aspect of the present invention, there is provided a compound, or a pharmaceutically acceptable salt, hydrate or solvate thereof, as defined herein for use in the treatment of a disease or disorder in which adenosine A2a is implicated.

According to a further aspect of the present invention, there is provided the use of a compound, or a pharmaceutically acceptable salt, hydrate or solvate thereof, as defined herein in the manufacture of a medicament for the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for the treatment of a disease or disorder in which adenosine A2a is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

The term “(m-nC)cycloalkyl” means a hydrocarbon ring containing from m to n carbon atoms, for example “(3-6C)cycloalkyl” means a hydrocarbon ring containing from 3 to 6 carbon atoms, for example, cyclopropyl, cyclobutyl, cyclopentyl, cyclohexyl. The term “(m-nC)-cycloalkyl” also encompasses non-aromatic saturated or partially saturated monocyclic, fused, bridged, or spiro bicyclic carbocyclic ring system(s). The term “(m-nC)cycloalkyl” includes both monovalent species and divalent species. Monocyclic “(m-nC)cycloalkyl” rings contain from about 3 to 12 (suitably from 3 to 8, most suitably from 5 to 6) ring carbon atoms. Bicyclic “(m-nC) cycloalkyl” contain from 7 to 17 ring carbon atoms, suitably 7 to 12 ring carbon atoms. Bicyclic “Cₘ₋ₙcycloalkyl” rings may be fused, spiro, or bridged ring systems.

“(3-8C)cycloalkyl” means a hydrocarbon ring or bridged ring system containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

A “carbon-linked heterocyclyl” means a heretocycle group as defined above that is connected via a carbon atom, rather than a heteroatom such as nitrogen.

By “spirocyclic ring systems” it is meant a compound which at least two rings which have only one atom in common and are not linked by a bridge.

By “fused ring systems” it is meant a compound in which two rings share two adjacent atoms. In other words, the rings share one covalent bond.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include κ-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular Examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular Examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For Example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention relates to compounds, or pharmaceutically acceptable salts, hydrates or solvates thereof, having the structural formula I shown below:

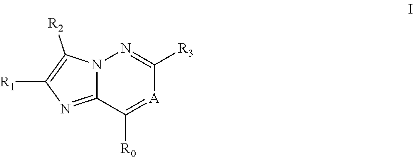

wherein:
R₀ is hydrogen or deuterium;
R₁ is selected from aryl or heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, (CH₂)_{q}1N(R_{1C})C(O)R_{1B}, (CH₂)_{q1}S(O)ₚR_{1B} (where p is 0, 1 or 2), (CH₂)_{q1}SO₂N(R_{1C})R_{1B}, or (CH₂)_{q}1N(R_{1C})SO₂R_{1B},
   and wherein q1 is 0, 1, 2 or 3 and R_{1B} and R_{1C} are each independently selected from hydrogen, (1-4C)alkyl, (3-6C)cycloalkyl or (3-6C)cycloalkyl(1-2C)alkyl;
R₂ is selected from hydrogen, cyano, halo, (1-4C)alkyl, (1-4C)haloalkyl, C(O)OR_{2A}, C(O)NR_{2A}R_{2B}, aryl, heterocyclyl, heteroaryl, (2-6C)alkenyl, (2-6C)alkynyl or (1-4C)alkanoyl;
   wherein R_{2A} and R_{2B} are each independently selected from hydrogen, (1-4C)alkyl, (1-4C)alkoxy, (3-6C)cycloalkyl or (3-6C)cycloalkyl(1-2C)alkyl, or, in the CONR_{2A}R_{2B} group, R_{2A} and R_{2B} are linked such that, together with the nitrogen atom to which they are attached, they form a heterocyclic ring, and
   wherein any alkyl, alkenyl, alkynyl, alkanoyl, aryl, heteroaryl or heterocyclyl group (formed by R_{2A} and R_{2B}) is optionally substituted by one or more substituents independently selected from oxo, (1-4C)alkyl, halo, (1-4C)haloalkyl, (1-4C)haloalkoxy, amino, (1-4C)aminoalkyl, cyano, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{2D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D}, wherein q2 is 0, 1, 2 or 3; and wherein R_{2D} and R_{2E} are each independently selected from hydrogen, (1-4C)alkyl, (3-6C)cycloalkyl or (3-6C)cycloalkyl(1-2C)alkyl;
R₃ is selected from hydrogen, halo, cyano or a group of the formula:

-L-Y-L_{q}-Q
wherein:
   L is absent or (1-4C)alkylene optionally substituted by one or more substituents selected from (1-2C)alkyl or oxo;
   Y is absent or O, S, SO, SO₂, N(Rₐ), C(O), C(O)O, OC(O), C(O)N(Rₐ), N(Rₐ)C(O), C(O)N(Rₐ)—O—, N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein
   R_{c}, R_{d} and Rₑ are each independently selected from hydrogen, (1-6C)alkyl, (3-6C)cycloalkyl or (3-6C)cycloalkyl(1-2C)alkyl; or
   R_{c} and R_{d} are linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy; and/or
   Q is optionally substituted by one or more group(s) of the formula:

-L₁-L_{Q1}-W₁
wherein:
   L₁ is absent or (1-3C)alkylene optionally substituted by one or more substituents selected from (1-2C)alkyl or oxo;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
   W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
   and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups; or
   Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
A is selected from CR₄ and N,
   wherein R₄ is hydrogen, halo or (1-4C)alkyl optionally substituted by one or more substituents selected from halo, (1-4C)haloalkyl, (1-4C)haloalkoxy, (1-4C)aminoalkyl, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and R_{4A} and R_{4B} are each independently selected from hydrogen, (1-6C)alkyl, (3-6C)cycloalkyl or (3-6C)cycloalkyl(1-2C)alkyl;
and wherein any tertiary amine in a compound of formula I is optionally in the form of a N-oxide and any nitrogen atom in a heteroaryl ring is optionally in the form of an N-oxide;
and wherein any S atoms present in the heterocyclic ring may optionally be present as S(═O), S(═O)₂ or S(═O)(═NR_{z}) wherein R_{z} is selected from hydrogen, (1-3C)alkyl or (2-3C)alkanoyl.

Particular compounds of the invention include, for example, compounds of the formula I, or pharmaceutically acceptable salts, hydrates and/or solvates thereof, wherein, unless otherwise stated, each of Rₒ, R₁, R₂, R₃ and A have any of the meanings defined hereinbefore or in any of paragraphs (1) to (54) hereinafter:—
(1) R₀ is hydrogen;
(2) R₀ is deuterium;
(3) R₁ is selected from aryl or heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, (CH₂)_{q1}N(R_{1C})C(O)R_{1B}, (CH₂)_{q1}S(O)ₚR_{1B} (where p is 0, 1 or 2), (CH₂)_{q1}SO₂N(R_{1C})R_{1B}, or (CH₂)_{q1}N(R_{1C})SO₂R_{1B},
   and wherein q1 is 0, 1, 2 or 3 and R_{1B} and R_{1C} are each independently selected from hydrogen, (1-2C)alkyl, (3-4C)cycloalkyl or (3-4C)cycloalkyl(1-2C)alkyl;
(4) R₁ is selected from aryl or heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
   q1 is 0, 1 or 2; and
   R_{1B} and R_{1C} are each independently selected from hydrogen, (1-4C)alkyl, (3-6C)cycloalkyl or (3-6C)cycloalkyl(1-2C)alkyl;
(5) R₁ is selected from aryl or heteroaryl,
   wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
   q1 is 0, 1 or 2; and R_{1B} and R_{1C} are each independently selected from hydrogen, (1-2C)alkyl or (3-4C)cycloalkyl;
(5a) R₁ is selected from aryl or heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, (CH₂)_{q}1N(R_{1C})C(O)R_{1B}, (CH₂)_{q}1S(O)ₚR_{1B} (where p is 0, 1 or 2), (CH₂)_{q}1SO₂N(R_{1C})R_{1B}, or (CH₂)_{q}1N(R_{1C})SO₂R_{1B} and wherein:
   q1 is 0, 1 or 2; and
   R_{1B} and R_{1C} are each independently selected from hydrogen, (1-4C)alkyl, (3-6C)cycloalkyl or (3-6C)cycloalkyl(1-2C)alkyl;
(6) R₁ is selected from phenyl or a 5 or 6 membered heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
   q1 is 0, 1 or 2; and
   R_{1B} and R_{1C} are each independently selected from hydrogen, (1-4C)alkyl, (3-6C)cycloalkyl or (3-6C)cycloalkyl(1-2C)alkyl;
(7) R₁ is selected from aryl or heteroaryl,
   wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
   q1 is 0, 1 or 2; and
   R_{1B} and R_{1C} are each independently selected from hydrogen, (1-2C)alkyl or (3-4C)cycloalkyl;
(8) R₁ is selected from aryl or heteroaryl,
   wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, or (CH₂)_{q1}N(R_{1C})C(O)R_{1B},
   and wherein q1 is 0, 1, 2 or 3 and R_{1B} and R_{1C} are each independently selected from hydrogen or (1-2C)alkyl;
(9) R₁ is selected from aryl or heteroaryl,
   wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, (CH₂)_{q1}OR_{1B}, or (CH₂)_{q1}C(O)R_{1B},
   and wherein q1 is 0, 1, 2 or 3 and R_{1B} and R_{1C} are each independently selected from hydrogen or (1-2C)alkyl;
(10) R₁ is selected from phenyl or a 5- or 6-membered heteroaryl,
   wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
   q1 is 0, 1 or 2; and
   R_{1B} and R_{1C} are each independently selected from hydrogen or (1-2C)alkyl;
(11) R₁ is phenyl, which is optionally substituted by one or more R_{1z} substituents defined in any one of paragraphs (1) to (8) above.
(12) R₁ is a 5 or 6-membered heteroaryl, which is optionally substituted by one or more R_{1z} substituents defined in any one of paragraphs (1) to (8) above.
(13) R₁ is selected from phenyl, furyl, pyridyl, oxazolyl, thiazolyl, isoxazolyl or oxazolin-2-yl, wherein a phenyl, furyl, pyridyl or oxazolyl ring is optionally substituted by halo, (1-2C)alkyl, (1-2C)alkoxy or cyano.
(14) R₁ is selected from phenyl, furyl, pyridyl or oxazolyl, wherein a phenyl, furyl, pyridyl or oxazolyl ring is optionally substituted by one or more of halo, C₁₋₂alkoxy or cyano.
(15) R₁ is selected from phenyl, furyl, pyridyl or oxazolyl, wherein a phenyl, furyl, pyridyl or oxazolyl ring is optionally substituted by halo or cyano.
(16) R₁ is selected from 3-cyanophenyl, furyl, or oxazolyl, thiazolyl, isoxazolyl or oxazolin-2-yl.
(17) R₁ is 3-cyanophenyl.
(18) R₂ is selected from hydrogen, cyano, halo, (1-4C)alkyl, (1-4C)haloalkyl, C(O)OR_{2A}, C(O)NR_{2A}R_{2B}, aryl, heterocyclyl, heteroaryl, (2-6C)alkenyl, (2-6C)alkynyl or (1-4C)alkanoyl;
   wherein R_{2A} and R_{2B} are each independently selected from hydrogen, (1-4C)alkyl, (1-4C)alkoxy, (3-6C)cycloalkyl or (3-6C)cycloalkyl(1-2C)alkyl,
   or, in the CONR_{2A}R_{2B} group, R_{2A} and R_{2B} are linked such that, together with the nitrogen atom to which they are attached, they form a 4-7 membered heterocyclic ring, and
   wherein any alkyl, alkenyl, alkynyl, alkanoyl, aryl, heteroaryl or heterocyclyl group (formed by R_{2A} and R_{2B}) is optionally substituted by one or more substituents independently selected from (1-4C)alkyl, halo, (1-2C)haloalkyl, (1-2C)haloalkoxy, cyano, (CH₂)_{q2}NR_{2S}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{12D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D},
   wherein q2 is 0, 1, or 2; and
   wherein R_{2D} and R_{2E} are each independently selected from hydrogen, (1-2C)alkyl, (3-4C)cycloalkyl or (3-4C)cycloalkyl(1-2C)alkyl;
(19) R₂ is selected from hydrogen, cyano, halo, (1-4C)alkyl, (1-4C)haloalkyl, C(O)OR_{2A}, C(O)NR_{2A}R_{2B}, phenyl, a 5 or 6-membered heteroaryl, a bicyclic heteroaryl, a 5 or 6-membered heterocyclyl, (2-4C)alkenyl or (1-4C)alkanoyl,
   wherein R_{2A} and R_{2B} are each independently selected from hydrogen, (1-4C)alkyl or (3-6C)cycloalkyl or (3-6C)cycloalkyl(1-2C)alkyl, and
   wherein any alkyl, alkenyl, alkanoyl, phenyl or heteroaryl group is optionally substituted by one or more substituents independently selected from (1-4C)alkyl, halo, (1-2C)haloalkyl, (1-2C)haloalkoxy, cyano, oxo, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{12D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D},
   wherein q2 is 0, 1, or 2;
   and wherein R_{2D} and R_{2E} are each independently selected from hydrogen, (1-2C)alkyl, (3-4C)cycloalkyl or (3-4C)cycloalkyl(1-2C)alkyl;
(20) R₂ is selected from hydrogen, cyano, halo, (1-2C)alkyl, (1-2C)haloalkyl, C(O)OR_{2A}, C(O)NR_{2A}R_{2B}, phenyl, a 5 or 6-membered heteroaryl, a bicyclic heteroaryl, a 5 or 6-membered heterocyclyl, or (1-4C)alkanoyl,
   wherein R_{2A} and R_{2B} are each independently selected from hydrogen or (1-4C)alkyl, and
   wherein any alkyl, alkenyl, alkanoyl, phenyl or heteroaryl group is optionally substituted by one or more substituents independently selected from (1-4C)alkyl, halo, (1-2C)haloalkyl, (1-2C)haloalkoxy, cyano, oxo, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{12D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D},
   wherein q2 is 0, 1, or 2; and
   wherein R_{2D} and R_{2E} are each independently selected from hydrogen, (1-2C)alkyl, (3-4C)cycloalkyl or (3-4C)cycloalkyl(1-2C)alkyl;
(21) R₂ is selected from cyano, halo, methyl, CF₃, C(O)OR_{2A}, C(O)NR_{2A}R_{2B}, a 5 or 6-membered heteroaryl, a 5 or 6-membered heterocyclyl, a bicyclic heteroaryl or (2-4C)alkanoyl,
   wherein R_{2A} and R_{2B} are each independently selected from hydrogen or (1-4C)alkyl, wherein any phenyl or heteroaryl group is optionally substituted by one or more substituents independently selected from (1-2C)alkyl, halo, (1-2C)haloalkyl, (1-2C)haloalkoxy, cyano, oxo, (CH₂)_{q2}NR_{2D}R_{2E}, (CH₂)_{q2}OR_{2D}, OR_{2D}, C(O)R_{2D}, C(O)OR_{2D}, OC(O)R_{2D}, C(O)N(R_{2E})R_{2D}, N(R_{2E})C(O)R_{12D}, S(O)ₚR_{2D} (where p is 0, 1 or 2), SO₂N(R_{2E})R_{2D}, or N(R_{2E})SO₂R_{2D}, wherein q2 is 0 or 1; and wherein R_{2D} and R_{2E} are each independently selected from hydrogen or (1-2C)alkyl;
(22) R₂ is selected from cyano, a 5 or 6-membered heteroaryl or a bicyclic heteroaryl, wherein the 5 or 6-membered heteroaryl or bicyclic heteroaryl is optionally substituted as defined above in any one of paragraphs (18) to (22);
(23) R₂ is selected from cyano, a 5 or 6-membered heteroaryl (e.g. pyridin4-yl) or a bicyclic heteroaryl, wherein the 5 or 6-membered heteroaryl or bicyclic heteroaryl is optionally substituted by one or more substituents independently selected from (1-2C)alkyl, halo, (1-2C)haloalkyl, (1-2C)alkoxy, (1-2C)haloalkoxy, (1-2C)hydroxyalkyl, (1-2C)alkanoyl or cyano;
(24) R₂ is a 5 or 6-membered heteroaryl (e.g. pyridin4-yl) or a bicyclic heteroaryl, wherein the 5 or 6-membered heteroaryl or bicyclic heteroaryl is optionally substituted by one or more substituents independently selected from (1-2C)alkyl, halo, (1-2C)haloalkyl, (1-2C)alkoxy, (1-2C)haloalkoxy, (1-2C)hydroxyalkyl, (2C)alkanoyl or cyano;
(25) R₂ is a 5 or 6-membered heteroaryl (e.g. pyridin4-yl) or a bicyclic heteroaryl, wherein the 5 or 6-membered heteroaryl or bicyclic heteroaryl is optionally substituted by one or more substituents independently selected from (1-2C)alkyl, (1-2C)hydroxyalkyl, or halo;
(26) R₂ is a 6-membered heteroaryl (e.g. pyridin4-yl) which is optionally substituted by one or more substituents independently selected from (1-2C)alkyl, halo, (1-2C)haloalkyl, (1-2C)alkoxy, (1-2C)haloalkoxy, (1-2C)hydroxyalkyl, (2C)alkanoyl or cyano;
(26a) R₂ is a 6-membered heteroaryl (e.g. pyridin4-yl) or a bicyclic heteroaryl (e.g. quinazolin-6-yl), wherein the 5 or 6-membered heteroaryl or bicyclic heteroaryl is optionally substituted by one or more substituents independently selected from methyl (including CD₃), methoxy, acetyl, difluoromethyl, trifluoromethyl, hydroxymethyl, or cyano;
(27) R₂ is a 6-membered nitrogen containing heteroaryl (e.g. pyridin4-yl) which is optionally substituted by one or more substituents independently selected from (1-2C)alkyl or halo;
(28) R₂ is a 6-membered nitrogen containing heteroaryl (e.g. pyridin4-yl) which is optionally substituted by one or more substituents independently selected from methyl (including CD₃) or chloro;
(29) R₂ is either:
A)

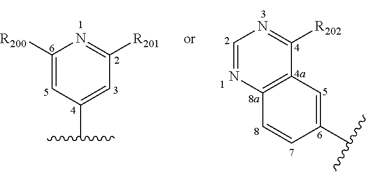

wherein:
(i) R₂₀₀ and R₂₀₁ are each independently selected from (1-2C)alkyl, halo, (1-2C)haloalkyl, (1-2C)alkoxy, (1-2C)haloalkoxy, (1-2C)hydroxyalkyl, (1-2C)alkanoyl or cyano; and
   R₂₀₂ is selected from (1-2C)alkyl, halo, (1-2C)haloalkyl, (1-2C)alkoxy, (1-2C)haloalkoxy, (1-2C)hydroxyalkyl, (1-2C)alkanoyl or cyano;
(ii) R₂₀₀ and R₂₀₁ are each independently selected from methyl (including CD₃), halo, di-fluoromethyl, trifluoromethyl, methoxy, hydroxymethyl, acetyl or cyano; and
   R₂₀₂ is selected from methyl (including CD₃), halo, di-fluoromethyl, trifluoromethyl, methoxy, hydroxymethyl, acetyl or cyano;
(iii) R₂₀₀ is methyl (including CD₃) or chloro and R₂₀₁ is selected from methyl (including CD₃), halo, di-fluoromethyl, trifluoromethyl, methoxy, hydroxymethyl, acetyl or cyano; and
   R₂₀₂ is methyl or chloro
(iv) R₂₀₀ is methyl (including CD₃) or chloro and R₂₀₁ is as defined in any of options (i) to (iii) above; and
   R₂₀₀ is methyl (including CD₃);
or
B)

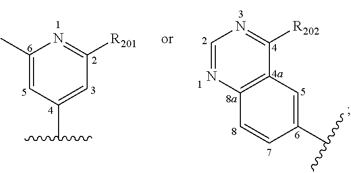

wherein:
(i) R₂₀₁ is (1-2C)alkyl, halo, (1-2C)haloalkyl, (1-2C)alkoxy, (1-2C)haloalkoxy, (1-2C)alkanoyl or cyano; and
   R₂₀₂ is (1-2C)alkyl, halo, (1-2C)haloalkyl, (1-2C)alkoxy, (1-2C)haloalkoxy, (1-2C)alkanoyl or cyano;
(ii) R₂₀₁ is methyl (including CD₃), halo, di-fluoromethyl, trifluoromethyl, methoxy, acetyl or cyano;
   R₂₀₂ is methyl (including CD₃), halo, di-fluoromethyl, trifluoromethyl, methoxy, acetyl or cyano;
(iii) R₂₀₁ is methyl (including CD₃) or chloro;
   R₂₀₂ is methyl (including CD₃) or chloro;
(iv) R₂₀₁ is methyl (including CD₃);
   R₂₀₂ is methyl (including CD₃); or
(v) R₂₀₁ is chloro
   R₂₀₂ is chloro;
(29a) R₂ is:

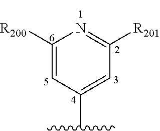

wherein:
(i) R₂₀₀ and R₂₀₁ are each independently selected from (1-2C)alkyl, halo, (1-2C)haloalkyl, (1-2C)alkoxy, (1-2C)haloalkoxy, (1-2C)hydroxyalkyl, (1-2C)alkanoyl or cyano;
(ii) R₂₀₀ and R₂₀₁ are each independently selected from methyl (including CD₃), halo, di-fluoromethyl, trifluoromethyl, methoxy, hydroxymethyl, acetyl or cyano;
(iii) R₂₀₀ is methyl or chloro and R₂₀₁ is selected from methyl (including CD₃), halo, di-fluoromethyl, trifluoromethyl, methoxy, hydroxymethyl, acetyl or cyano;
(iv) R₂₀₀ is methyl (including CD₃) or chloro and R₂₀₁ is as defined in any of options (i) to (iii) above;
or

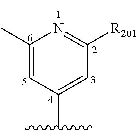

wherein:
(i) R₂₀₁ is (1-2C)alkyl, halo, (1-2C)haloalkyl, (1-2C)alkoxy, (1-2C)haloalkoxy, (1-2C)alkanoyl or cyano;
(ii) R₂₀₁ is methyl (including CD₃), halo, di-fluoromethyl, trifluoromethyl, methoxy, acetyl or cyano;
(iii) R₂₀₁ is methyl (including CD₃) or chloro;
(iv) R₂₀₁ is methyl (including CD₃); or
(v) R₂₀₁ is chloro;
(30) R₂ is:
   bromo;
   2-acetyl-6-methylpyridin-4-yl;
   2,6-dimethylpyridin-4-yl;
   2-Chloro-6-methylpyridin-4-yl
   2-methyl-6-(trifluoromethyl)pyridine-4-yl;
   2-methoxy-6-methyl-4-pyridyl;
   2-(difluoromethyl)-6-methyl-4-pyridyl;
   2-chloro-6-methyl-4-pyridyl;
   2-chloro-6-methylpyridin-4-yl or 2,6-dimethylpyridin-4-yl;
   2,6-bis(trideuteriomethyl)pyridyl;
   4-methylquinazolin-6-yl;
   2-(hydroxymethyl)-6-methyl-4-pyridyl
(30a) R₂ is:

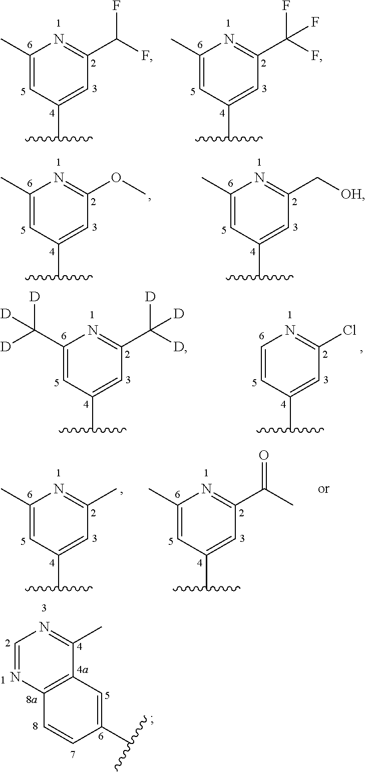

(30b) R₂ is 2-chloro-6-methylpyridin-4-yl or 2,6-dimethylpyridin-4-yl, i.e.

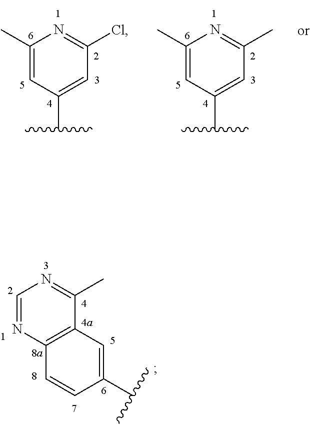

(30c) R₂ is 2-chloro-6-methylpyridin-4-yl or 2,6-dimethylpyridin-4-yl, i.e.

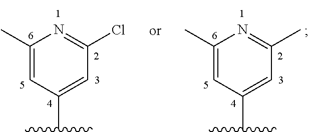

(31) R₃ is selected from hydrogen, halo, cyano or a group of the formula:

-L-Y-L_{q}-Q
wherein:
   L is absent or (1-4C)alkylene;
   Y is absent or O, S, SO, SO₂, N(Rₐ), C(O), C(O)O, OC(O), C(O)N(Rₐ), C(O)N(Rₐ)O, N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein
   R_{c} and R_{d} are each independently selected from hydrogen, (1-6C)alkyl, (3-6C)cycloalkyl or (3-6C)cycloalkyl(1-2C)alkyl; or R_{c} and R_{d} are linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy; and/or
   Q is optionally substituted by one or more group(s) of the formula:

-L₁-L_{Q1}-W₁
wherein:
   L₁ is absent or (1-3C)alkylene;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
   W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
   and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups; or
   Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
(32) R₃ is selected from hydrogen, halo, cyano or a group of the formula:

-L-Y-L_{q}-Q
wherein:
   L is absent or (1-4C)alkylene;
   Y is absent or O, S, SO, SO₂, N(Rₐ), C(O), C(O)O, OC(O), C(O)N(Rₐ), C(O)N(Rₐ)O, N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen, (1-6C)alkyl, (3-6C)cycloalkyl or (3-6C)cycloalkyl(1-2C)alkyl; and/or
   Q is optionally substituted by one or more group(s) of the formula:

-L₁-L_{Q1}-W₁
wherein:
   L₁ is absent or (1-3C)alkylene;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
   W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
(33) R₃ is selected from hydrogen, halo, cyano or a group of the formula:

-L-Y-L_{q}-Q
wherein:
   L is absent or (1-2C)alkylene;
   Y is absent or O, S, SO, SO₂, N(Rₐ), C(O), C(O)O, OC(O), C(O)N(Rₐ), C(O)N(Rₐ)O, N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, aryl, (3-8)cycloalkyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen, (1-6C)alkyl, (3-6C)cycloalkyl or (3-6C)cycloalkyl(1-2C)alkyl; and/or
   Q is optionally substituted by one or more group(s) of the formula:

-L₁-L_{Q1}-W₁
wherein:
   L₁ is absent or (1-2C)alkylene;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
   W₁ is hydrogen, (1-6C)alkyl, aryl, aryl(1-2C)alkyl, (3-8C)cycloalkyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen or (1-4C)alkyl;
(34) R₃ is selected from hydrogen, halo, cyano or a group of the formula:

-L-Y-L_{q}-Q
wherein:
   L is absent or (1-2C)alkylene;
   Y is absent or O, N(Rₐ), C(O), C(O)O, C(O)N(Rₐ), N(Rₐ)C(O), N(Rₐ)C(O)N(R_{b}), C(O)N(Rₐ)O, N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, aryl, (3-8)cycloalkyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen or (1-6C)alkyl; and/or
   Q is optionally substituted by one or more group(s) of the formula:

-L₁-L_{Q1}-W₁
wherein:
   L₁ is absent or (1-2C)alkylene;
   L_{Q1} is absent; and
   W₁ is hydrogen, (1-6C)alkyl, aryl, aryl(1-2C)alkyl, (3-8C)cycloalkyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), wherein Rₕ and Rᵢ are each independently selected from hydrogen or (1-4C)alkyl;
(35) R₃ is a group of the formula:

-L-Y-L_{q}-Q
wherein:
   L is absent;
   Y is N(Rₐ) or C(O)N(Rₐ);
   L_{q} is absent; and
   Q is (1-6C)alkyl or (3-8)cycloalkyl;
   wherein Q is optionally further substituted by one or more substituent groups independently selected from halo, cyano, NR_{c}R_{d}, OR_{c}, C(O)R_{c}, C(O)OR_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen or (1-6C)alkyl;
(36) R₃ is a group of the formula:

-L-Y-L_{q}-Q
wherein:
   L is absent;
   Y is N(Rₐ) or C(O)N(Rₐ);
   L_{q} is absent; and
   Q is (1-6C)alkyl;
   wherein Q is optionally further substituted by one or more substituent groups independently selected from halo, cyano, NR_{c}R_{d}, OR_{c}, C(O)OR_{c}, S(O)ₚR_{c} (where p is 0, 1 or 2), SO₂N(R_{d})R_{c}; wherein R_{c} and R_{d} are each independently selected from hydrogen or (1-6C)alkyl;
(37) R₃ is a group of the formula:

-L-Y-L_{q}-Q
wherein:
   L is absent;
   Y is N(Rₐ) or C(O)N(Rₐ);
   L_{q} is absent; and
   Q is (1-6C)alkyl;
   wherein Q is optionally further substituted by one or more OR_{c}; wherein R_{c} is selected from hydrogen or (1-4C)alkyl;
(38) R₃ is a group of the formula:

-L-Y-L_{q}-Q
wherein:
   L is absent;
   Y is N(Rₐ) or C(O)N(Rₐ);
   L_{q} is absent; and
   Q is (1-6C)alkyl;
   wherein Q is optionally further substituted by one or more OH;
(39) R₃ is a group of the formula:

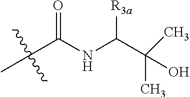

wherein R₃ₐ is hydrogen or methyl;
(40) R₃ is selected from halo or a group of the formula:

-L-Y-Q
wherein:
   L is absent;
   Y is absent or O, S, SO, SO₂, N(Rₐ), C(O), C(O)O, OC(O), C(O)N(Rₐ), C(O)N(Rₐ)O, N(Rₐ)C(O), S(O)₂N(Rₐ), N(Rₐ)SO₂N(R_{b}), or N(Rₐ)SO₂, wherein Rₐ and R_{b} are each independently selected from hydrogen or (1-2C)alkyl; and
   Q is (1-4C)alkyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c}, R_{d} and Rₑ are each independently selected from hydrogen, (1-4C)alkyl or (3-6C)cycloalkyl; or
   R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy; and/or
   Q is optionally substituted by a group of the formula:

-L₁-L_{Q1}-W₁
wherein:
L₁ is absent or (1-3C)alkylene optionally substituted by one or more substituents selected from (1-2C)alkyl or oxo;
L_{Q1} is absent or selected from or O, S, SO, SO₂, N(R_{f}), C(O), C(O)O, OC(O), C(O)N(R_{f}), N(R_{f})C(O), N(R_{f})C(O)O, OC(O)N(R_{f}), S(O)₂N(R_{f}), or N(R_{f})SO₂, wherein R_{f} and R_{g} are each independently selected from hydrogen or (1-2C)alkyl; and
W₁ is hydrogen, (1-4C)alkyl, aryl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl or (3-6C)cycloalkyl; and wherein any alkyl, alkoxy, aryl, heretoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups;
and wherein any tertiary amine present in a R₃ group is optionally in the form of a N-oxide;
(41) R₃ is selected from halo or a group of the formula:

-Q
wherein:
   Q is heteroaryl or heterocyclyl;
   wherein Q is optionally further substituted by one or more substituent groups independently selected from oxo, (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c}, R_{d} and Rₑ are each independently selected from hydrogen, (1-4C)alkyl or (3-6C)cycloalkyl; or
   R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy; and/or
   Q is optionally substituted by a group of the formula:

-L₁-L_{Q1}-W₁
wherein:
L₁ is absent or (1-3C)alkylene optionally substituted by one or more substituents selected from (1-2C)alkyl or oxo;
L_{Q1} is absent or selected from or O, S, SO, SO₂, N(R_{f}), C(O), C(O)O, OC(O), C(O)N(R_{f}), N(R_{f})C(O), N(R_{f})C(O)O, OC(O)N(R_{f}), S(O)₂N(R_{f}), or N(R_{f})SO₂, wherein R_{f} and R_{g} are each independently selected from hydrogen or (1-2C)alkyl; and
W₁ is hydrogen, (1-4C)alkyl, aryl, heteroaryl or heterocyclyl;
wherein W₁ is optionally substituted by one or more substituents selected from oxo, (1-4C)alkyl, halo, (1-4C)haloalkyl, (1-4C)haloalkoxy, (1-4C)alkoxy, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl or (3-6C)cycloalkyl;
or Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups;
and wherein any tertiary amine present in a R₃ group is optionally in the form of a N-oxide;
(42) R₃ is a heterocyclyl which is optionally further substituted by one or more substituent groups independently selected from oxo, (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c}, R_{d} and Rₑ are each independently selected from hydrogen, (1-4C)alkyl or (3-6C)cycloalkyl; or
   R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy; and/or
   R₃ is optionally substituted by a group of the formula:

-L₁-L_{Q1}-W₁
wherein:
L₁ is absent or (1-3C)alkylene optionally substituted by one or more substituents selected from (1-2C)alkyl or oxo;
L_{Q1} is absent or selected from or O, S, SO, SO₂, N(R_{f}), C(O), C(O)O, OC(O), C(O)N(R_{f}), N(R_{f})C(O), N(R_{f})C(O)O, OC(O)N(R_{f}), S(O)₂N(R_{f}), or N(R_{f})SO₂, wherein R_{f} and R_{g} are each independently selected from hydrogen or (1-2C)alkyl; and
W₁ is hydrogen, (1-4C)alkyl, aryl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl or (3-6C)cycloalkyl; or Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups;
and wherein any tertiary amine present in a R₃ group is optionally in the form of a N-oxide;
(43) R₃ is a nitrogen-linked heterocycle selected from a 4-7 membered heterocylic ring system, a 9-15 membered bicyclic ring system or a 9-15 membered spirocyclic ring system;
   wherein R₃ optionally further substituted by one or more substituent groups independently selected from oxo, (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c}, R_{d} and Rₑ are each independently selected from hydrogen, (1-4C)alkyl or (3-6C)cycloalkyl; or
      R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy; and/or
   R₃ is optionally substituted by a group of the formula:

-L₁-L_{Q1}-W₁
wherein:
L₁ is absent or (1-3C)alkylene optionally substituted by one or more substituents selected from (1-2C)alkyl or oxo;
L_{Q1} is absent or selected from or O, S, SO, SO₂, N(R_{f}), C(O), C(O)O, OC(O), C(O)N(R_{f}), N(R_{f})C(O), N(R_{f})C(O)O, OC(O)N(R_{f}), S(O)₂N(R_{f}), or N(R_{f})SO₂, wherein R_{f} and R_{g} are each independently selected from hydrogen or (1-2C)alkyl; and
W₁ is hydrogen, (1-4C)alkyl, aryl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl or (3-6C)cycloalkyl; or Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups;
and wherein any tertiary amine present in a R₃ group is optionally in the form of a N-oxide.
(44) R₃ is a heterocyclyl selected from piperazinyl, piperidinyl, pyrrolidinyl, oxetanyl, morpholinyl, diazepanyl, azetidinyl, each of which may be optionally further substituted by one or more R₆ groups; or R₃ has one of the following structures:

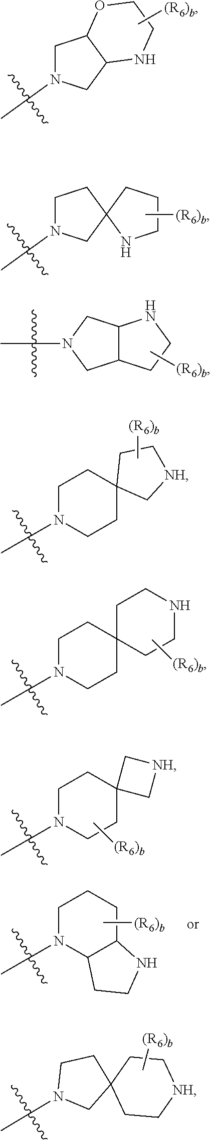

wherein b is an integer selected from 0, 1, 2, 3 or 4;
wherein each R₆ group is independently selected from (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3) or a group of the formula:

-L₁-L_{Q1}-W₁
wherein R_{c}, R_{d} and Rₑ are each independently selected from hydrogen, (1-4C)alkyl or (3-6C)cycloalkyl; or R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy; and
wherein:
   L₁ is absent or (1-3C)alkylene optionally substituted by one or more substituents selected from (1-2C)alkyl or oxo;
   L_{Q1} is absent or selected from or O, S, SO, SO₂, N(R_{f}), C(O), C(O)O, OC(O), C(O)N(R_{f}), N(R_{f})C(O), N(R_{f})C(O)O, OC(O)N(R_{f}), S(O)₂N(R_{f}), or N(R_{f})SO₂, wherein R_{f} and R_{g} are each independently selected from hydrogen or (1-2C)alkyl; and
   W₁ is hydrogen, (1-4C)alkyl, aryl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl or (3-6C)cycloalkyl; or Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
   and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups;
and wherein any tertiary amine present in a R₃ group is optionally in the form of a N-oxide.
(45) R₃ is a heterocyclyl selected from piperazinyl, piperidinyl, pyrrolidinyl, oxetanyl, morpholinyl, diazepanyl, azetidinyl, or one of the following structures:

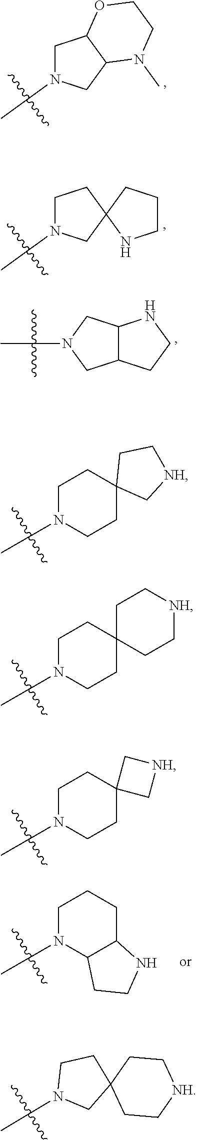

(46) R₃ is a nitrogen-linked heterocycle selected from a 4-7 membered heterocylic ring system, a 9-15 membered bicyclic ring system or a 9-15 membered spirocyclic ring system;
   wherein R₃ optionally further substituted by one or more substituent groups independently selected from oxo, (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3);
   wherein R_{c}, R_{d} and Rₑ are each independently selected from hydrogen, (1-4C)alkyl or (3-6C)cycloalkyl; or R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy.
(47) R₃ is a heterocyclyl selected from piperazinyl, piperidinyl, pyrrolidinyl, oxetanyl, morpholinyl, diazepanyl, azetidinyl, each of which may be optionally further substituted by one or more R₆ groups; or R₃ has one of the following structures:

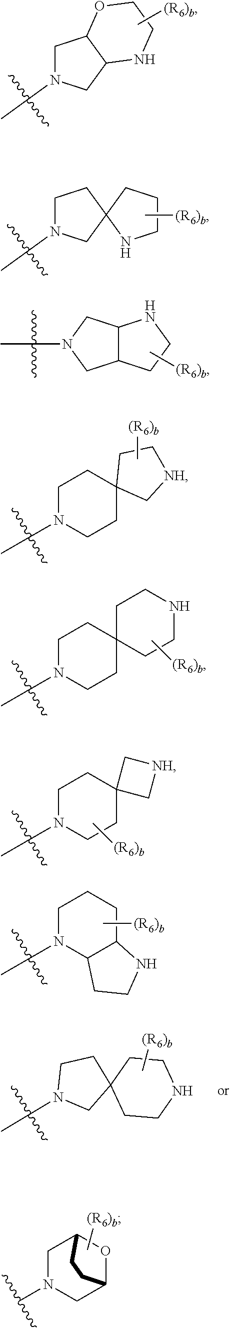

wherein b is an integer selected from 0, 1, 2, 3 or 4;
wherein each R₆ group is independently selected from (1-4C)alkyl, halo, (1-4C)haloalkyl, (1-4C)haloalkoxy, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3);
wherein R_{c} and R_{d} are each independently selected from hydrogen, (1-4C)alkyl or (3-6C)cycloalkyl; or R_{c} and R_{d} are linked such that, together with the nitrogen atom to which they are attached, they form a 4-6 membered heterocyclic ring which is optionally substituted by one or more substituents selected from (1-2C)alkyl, halo, (1-2C)haloalkyl, (1-2C)haloalkoxy, (1-2C)alkoxy, (1-2C)alkylamino, di-[(1-2C)alkyl]amino, amino, cyano or hydroxy;
and wherein any tertiary amine present in a R₃ group is optionally in the form of a N-oxide.
(48) R₃ is a heterocyclyl selected from piperazinyl, piperidinyl, pyrrolidinyl, oxetanyl, morpholinyl, diazepanyl, azetidinyl, or one of the following structures:

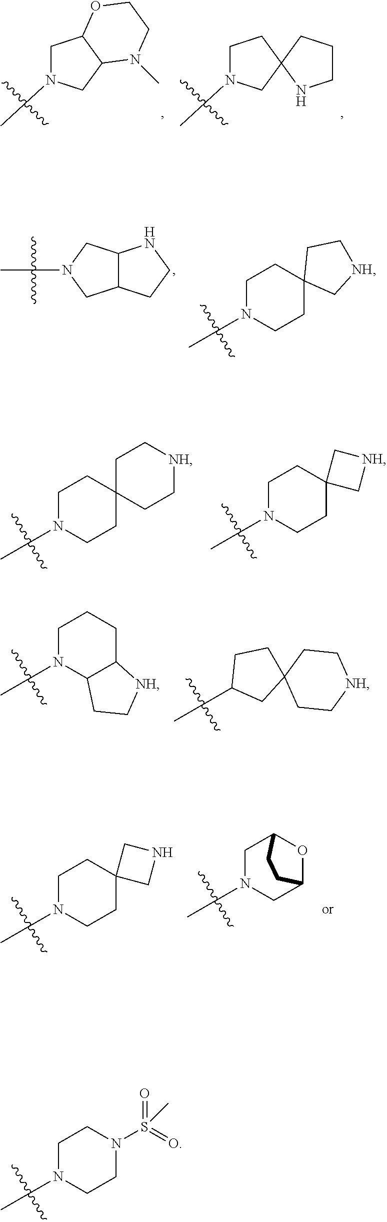

(49) A is selected from CR₄ and N,
   wherein R₄ is hydrogen, halo or (1-2C)alkyl optionally substituted by one or more substituents selected from halo, (1-2C)haloalkyl, (1-2C)haloalkoxy, amino, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and wherein R_{4A} and R_{4B} are each independently selected from hydrogen, (1-4C)alkyl, (3-4C)cycloalkyl or (3-4C)cycloalkyl(1-2C)alkyl;
(50) A is selected from CR₄ and N,
   wherein R₄ is hydrogen, halo or (1-2C)alkyl optionally substituted by one or more substituents selected from halo;
(51) A is selected from CR₄ and N,
   wherein R₄ is hydrogen, methyl or halo;
(52) A is from CR₄ and R₄ is hydrogen, methyl, fluoro or chloro;
(53) A is CH;
(54) A is N.

Suitably, a heteroaryl or heterocyclyl group as defined herein is a monocyclic heteroaryl or mono, bicyclic or bridged heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5-, 6-, 7- or 8-membered heterocyclyl ring comprising one, two or three heteroatoms selected from N, O or S. Most suitably, a heterocyclyl group is a 5-, 6- or 7-membered ring comprising one, two or three heteroatoms selected from N, O or S [e.g. morpholinyl (e.g. 4-morpholinyl), pyridinyl, piperazinyl, homopiperazinyl or pyrrolidinonyl].

Suitably, an aryl group is phenyl.

Suitably, R₀ is as defined in paragraphs (1) or (2) above. In an embodiment, R₀ is hydrogen. In an another embodiment, R₀ is deuterium.

Suitably, R₁ is as defined in any one of paragraphs (3) to (17) above. Most suitably, R₁ is as defined in paragraph (13) or (17) above.

Suitably, R₂ is as defined in any one of paragraphs (18) to (30c). More suitably, R₂ is as defined in any one of paragraphs (25) to (30c). Most suitably, R₂ is as defined in paragraph (25) or (30c).

Suitably, R₃ is as defined in any one of paragraphs (31) to (48). More suitably, R₃ is as defined in any one of paragraphs (34) to (39). Most suitably, R₃ is as defined in paragraph (37), (38) or (39).

Suitably, A is as defined in any one of paragraphs (49) to (54). Most suitably, A is as defined in paragraph (52) or (53).

In a particular group of compounds of Formula I above, R₁ is as defined in any one of paragraphs (3), (4), (5), (5a), (10), (13) or (17) and R₀, R₂, R₃ and A each have any one of the definitions herein.

In a particular group of compounds of Formula I above, R₂ is as defined in any one of paragraphs (18), (19), (20), (25), (26), (26a), (29), (29a), (30), (30a), (30b) or (30c) and R₀, R₁, R₃ and A each have any one of the definitions herein.

In a particular group of compounds of Formula I above, R₃ is as defined in any one of paragraphs (31), (32), (33), (34), (37), (38) or (39) and R₀, R₁, R₂ and A each have any one of the definitions herein.

In a particular group of compounds of Formula I above, A is as defined in any one of paragraphs (49), (50), (52) or (53) and R₀, R₁, R₂ and R₃ each have any one of the definitions herein.

In a particular group of compounds of the invention, the compounds have the structural formula Ib [a sub-definition of formula (I)] shown below, or a pharmaceutically acceptable salt, hydrate and/or solvate thereof:

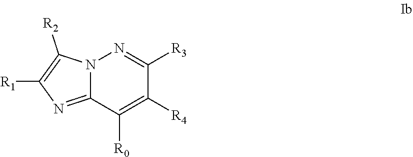

wherein R₀, R₁, R₂ and R₃ are each as defined hereinbefore and R₄ is hydrogen, methyl, fluoro or chloro.

In an embodiment of the compounds of formula Ib:
R₀ is as defined in either paragraph (1) or (2);
R₁ is as defined in any one of paragraphs (3) to (17) above;
R₂ is as defined in any one of paragraphs (18) to (30c) above;
R₃ is as defined in any one of paragraphs (31) to (48) above; and
R₄ is hydrogen, methyl, fluoro or chloro.

In an embodiment of the compounds of formula Ib:
R₀ is as defined in either paragraph (1) or (2);
R₁ is as defined in any one of paragraphs (3) to (17) above;
R₂ is as defined in any one of paragraphs (18) to (30c) above;
R₃ is as defined in any one of paragraphs (31) to (39) above; and
R₄ is hydrogen, methyl, fluoro or chloro.

In another embodiment of the compounds of formula Ib:
R₀ is as defined in paragraph (1) above;
R₁ is as defined in paragraph (13) above;
R₂ is as defined in paragraph (25) above;
R₃ is as defined in paragraph (34) above; and
R₄ is hydrogen.

In another embodiment of the compounds of formula Ib:
R₀ is as defined in paragraph (1) above;
R₁ is as defined in paragraph (13) above;
R₂ is as defined in paragraph (25) above;
R₃ is as defined in paragraph (32), (33) or (34) above; and
R₄ is hydrogen.

In another embodiment of the compounds of formula Ib:
R₀ is as defined in paragraph (1) above;
R₁ is as defined in paragraph (17) above;
R₂ is as defined in paragraph (30), (30a), (30b) or (30c) above;
R₃ is as defined in paragraph (39) above; and
R₄ is hydrogen.

In another embodiment of the compounds of formula Ib:
R₀ is as defined in paragraph (1) above;
R₁ is as defined in paragraph (17) above;
R₂ is as defined in paragraph (30c) above;
R₃ is as defined in paragraph (39) above; and
R₄ is hydrogen.

In a particular group of compounds of the invention, the compounds have the structural formula Ic [a sub-definition of formula (I)] shown below, or a pharmaceutically acceptable salt, hydrate and/or solvate thereof:

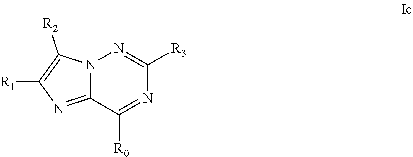

wherein R₀, R₁, R₂ and R₃ are each as defined hereinbefore.

In an embodiment of the compounds of formula Ic:
R₀ is as defined in either paragraph (1) or (2);
R₁ is as defined in any one of paragraphs (3) to (17) above;
R₂ is as defined in any one of paragraphs (18) to (30c) above; and
R₃ is as defined in any one of paragraphs (31) to (48) above.

In an embodiment of the compounds of formula Ic:
R₀ is as defined in either paragraph (1) or (2);
R₁ is as defined in any one of paragraphs (3) to (17) above;
R₂ is as defined in any one of paragraphs (18) to (30c) above; and
R₃ is as defined in any one of paragraphs (31) to (39) above.

In another embodiment of the compounds of formula Ic:
R₀ is as defined in paragraph (1) above;
R₁ is as defined in paragraph (13) above;
R₂ is as defined in paragraph (25) above; and
R₃ is as defined in paragraph (34) above.

In another embodiment of the compounds of formula Ic:
R₁ is as defined in paragraph (13) above;
R₂ is as defined in paragraph (26) or (26a) above;
R₃ is as defined in paragraph (34) above; and
A is as defined in paragraph (50) above.

In another embodiment of the compounds of formula Ic:
R₀ is as defined in paragraph (1) above;
R₁ is as defined in paragraph (17) above;
R₂ is as defined in paragraph (30c) above; and
R₃ is as defined in paragraph (37) or (38) above.

In another embodiment of the compounds of formula Ic:
R₀ is as defined in paragraph (1) above;
R₁ is as defined in paragraph (17) above;
R₂ is as defined in paragraph (30c) above; and
R₃ is as defined in paragraph (39) above.

In another embodiment of the compounds of formula Ic:
R₀ is as defined in paragraph (1) above;
R₁ is as defined in paragraph (17) above;
R₂ is as defined in paragraph (30), (30a), (30b) or (30c) above; and
R₃ is as defined in paragraph (39) above.

In a particular group of compounds of the invention, the compounds have the structural formula Id [a sub-definition of formula (I)] shown below, or a pharmaceutically acceptable salt, hydrate and/or solvate thereof:

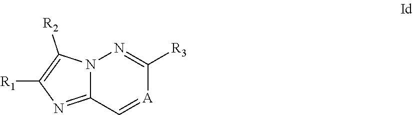

wherein A, R₁, R₂ and R₃ are each as defined hereinbefore.

In an embodiment of the compounds of formula Id:
R₁ is as defined in any one of paragraphs (3) to (17) above;
R₂ is as defined in any one of paragraphs (18) to (30c) above;
R₃ is as defined in any one of paragraphs (31) to (48) above; and
A is as defined in any one of paragraphs (49) to (54) above.

In an embodiment of the compounds of formula Id:
R₁ is as defined in any one of paragraphs (3) to (17) above;
R₂ is as defined in any one of paragraphs (18) to (30c) above;
R₃ is as defined in any one of paragraphs (31) to (39) above; and
A is as defined in any one of paragraphs (49) to (54) above.

In another embodiment of the compounds of formula Id:
R₁ is as defined in paragraph (13) above;
R₂ is as defined in paragraph (25) above;
R₃ is as defined in paragraph (34) above; and
A is as defined in paragraph (50) above.

In another embodiment of the compounds of formula Id:
R₁ is as defined in paragraph (13) above;
R₂ is as defined in paragraph (26) or (26a) above;
R₃ is as defined in paragraph (34) above; and
A is as defined in paragraph (50) above.

In another embodiment of the compounds of formula Id:
R₁ is as defined in paragraph (17) above;
R₂ is as defined in paragraph (30c) above; and
R₃ is as defined in paragraph (37) or (38) above; and
A is as defined in paragraph (52) or (53) above.

In another embodiment of the compounds of formula Id:
R₁ is as defined in paragraph (17) above;
R₂ is as defined in paragraph (30), (30a), (30b) or (30c) above;
R₃ is as defined in paragraph (39) above; and
A is as defined in paragraph (52) or (53) above.

In another embodiment of the compounds of formula Id:
R₁ is as defined in paragraph (17) above;
R₂ is as defined in paragraph (30c) above; and
R₃ is as defined in paragraph (39) above; and
A is as defined in paragraph (52) or (53) above.

In a particular group of compounds of the invention, the compounds have the structural formula Ie [a sub-definition of formula (I)] shown below, or a pharmaceutically acceptable salt, hydrate and/or solvate thereof:

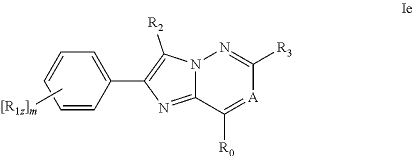

wherein A, R₀, R₂, R₃ and R_{1Z} are each as defined hereinbefore and m is 0, 1 or 2.

In an embodiment of the compounds of formula Ie:
R₀ is as defined in either paragraph (1) or (2) above;
R_{1z} is as defined in any one of paragraphs (3) to (11) above;
m is 0, 1 or 2;
R₂ is as defined in any one of paragraphs (18) to (30c) above;
R₃ is as defined in any one of paragraphs (31) to (48) above; and
A is as defined in any one of paragraphs (49) to (54) above.

In an embodiment of the compounds of formula Ie:
R₀ is as defined in either paragraph (1) or (2) above;
R_{1z} is as defined in any one of paragraphs (3) to (11) above;
m is 0, 1 or 2;
R₂ is as defined in any one of paragraphs (18) to (30c) above;
R₃ is as defined in any one of paragraphs (31) to (39) above; and
A is as defined in any one of paragraphs (49) to (54) above.

In another embodiment of the compounds of formula Ie:
R₀ is as defined in paragraph (1) above;
R_{1z} is halo or cyano;
m is 0 or 1;
R₂ is as defined in paragraph (25) above;
R₃ is as defined in paragraph (34) above; and
A is as defined in paragraph (50) above.

In another embodiment of the compounds of formula Ie:
R₀ is as defined in paragraph (1) above;
R_{1z} is cyano;
m is 1;
R₁ is as defined in paragraph (17) above;
R₂ is as defined in paragraph (30c) above;
R₃ is as defined in paragraph (37) or (38) above; and
A is as defined in paragraph (52) or (53) above.

In another embodiment of the compounds of formula Ie:
R₀ is as defined in paragraph (1) above;
R_{1z} is cyano;
m is 1;
R₁ is as defined in paragraph (17) above;
R₂ is as defined in paragraph (30), (30a), (30b) or (30c) above;
R₃ is as defined in paragraph (39) above; and
A is as defined in paragraph (52) or (53) above.

In another embodiment of the compounds of formula Ie:
R₀ is as defined in paragraph (1) above;
R_{1z} is cyano;
m is 1;
R₁ is as defined in paragraph (17) above;
R₂ is as defined in paragraph (30c) above;
R₃ is as defined in paragraph (39) above; and
A is as defined in paragraph (52) or (53) above.

In another embodiment of the compounds of formula Ie:
R₀ is as defined in paragraph (1) above;
R_{1z} is halo or cyano;
m is 1;
R₂ is as defined in paragraph (26) or (26a) above;
R₃ is as defined in paragraph (35) above; and
A is as defined in paragraph (50) above.

In a particular group of compounds of the invention, the compounds have the structural formula If [a sub-definition of formula (I)] shown below, or a pharmaceutically acceptable salt, hydrate and/or solvate thereof:

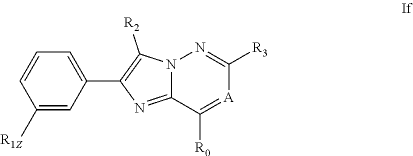

wherein A, R₀, R₂, R₃ and R_{1z} are each as defined hereinbefore.

In an embodiment of the compounds of formula If:
R₀ is as defined in either paragraph (1) or (2) above;
R_{1z} is as defined in any one of paragraphs (3) to (11) above;
R₂ is as defined in any one of paragraphs (18) to (30c) above;
R₃ is as defined in any one of paragraphs (31) to (48) above; and
A is as defined in any one of paragraphs (49) to (54) above.

In an embodiment of the compounds of formula If:
R₀ is as defined in either paragraph (1) or (2) above;
R_{1z} is as defined in any one of paragraphs (3) to (11) above;
R₂ is as defined in any one of paragraphs (18) to (30c) above;
R₃ is as defined in any one of paragraphs (31) to (39) above; and
A is as defined in any one of paragraphs (49) to (54) above.

In another embodiment of the compounds of formula If:
R₀ is as defined in paragraph (1) above;
R_{1z} is halo or cyano;
R₂ is as defined in paragraph (25) above;
R₃ is as defined in paragraph (34) above; and
A is as defined in paragraph (50) above.

In another embodiment of the compounds of formula If:
R₀ is as defined in paragraph (1) above;
R_{1z} is halo or cyano;
R₂ is as defined in paragraph (26) or (26a) above;
R₃ is as defined in paragraph (35) above; and
A is as defined in paragraph (50) above.

In another embodiment of the compounds of formula If:
R₀ is as defined in paragraph (1) above;
R_{1z} is cyano;
R₂ is as defined in paragraph (30), (30a), (30b) or (30c) above;
R₃ is as defined in paragraph (37) or (38) above; and
A is as defined in paragraph (52) or (53) above.

In another embodiment of the compounds of formula If:
R₀ is as defined in paragraph (1) above;
R_{1z} is cyano;
R₂ is as defined in paragraph (30c) above;
R₃ is as defined in paragraph (37) or (38) above; and
A is as defined in paragraph (52) or (53) above.

In another embodiment of the compounds of formula If:
R₀ is as defined in paragraph (1) above;
R_{1z} is cyano;
R₂ is as defined in paragraph (30) above;
R₃ is as defined in paragraph (39) above; and
A is as defined in paragraph (52) or (53) above.

In a particular group of compounds of the invention, the compounds have the structural formula Ig [a sub-definition of formula (I)] shown below, or a pharmaceutically acceptable salt, hydrate and/or solvate thereof:

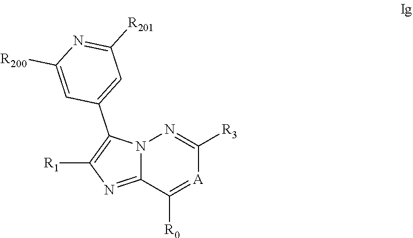

wherein A, R₀, R₁ and R₃ are each as defined hereinbefore; and R₂₀₀ and R₂₀₁ are each independently selected from hydrogen, methyl, halo, trifluoromethyl, difluoromethyl, methoxy or acetyl.

In an embodiment of the compounds of formula Ig:
R₀ is as defined in either paragraph (1) or (2) above;
R₁ is as defined in any one of paragraphs (3) to (17) above;
R₃ is as defined in any one of paragraphs (31) to (48) above;
A is as defined in any one of paragraphs (49) to (54) above; and
R₂₀₀ and R₂₀₁ are each independently selected from hydrogen, methyl (including CD₃) or halo.

In an embodiment of the compounds of formula Ig:
R₀ is as defined in either paragraph (1) or (2) above;
R₁ is as defined in any one of paragraphs (3) to (17) above;
R₃ is as defined in any one of paragraphs (31) to (39) above;
A is as defined in any one of paragraphs (49) to (54) above; and
R₂₀₀ and R₂₀₁ are each independently selected from hydrogen, methyl (including CD₃) or halo.

In another embodiment of the compounds of formula Ig:
R₀ is as defined in paragraph (1) above;
R₁ is as defined in paragraph (13) above;
R₃ is as defined in paragraph (34) above;
A is as defined in paragraph (50) above; and
R₂₀₀ and R₂₀₁ are each independently selected from methyl (including CD₃) or chloro.

In another embodiment of the compounds of formula Ig:
R₀ is as defined in paragraph (1) above;
R₁ is as defined in paragraph (17) above;
R₃ is as defined in paragraph (37) or (38) above;
A is as defined in paragraph (52) or (53) above; and
R₂₀₀ is chloro and R₂₀₁ is methyl (including CD₃).

In another embodiment of the compounds of formula Ig:
R₀ is as defined in paragraph (1) above;
R₁ is as defined in paragraph (17) above;
R₃ is as defined in paragraph (39) above;
A is as defined in paragraph (52) or (53) above; and
R₂₀₀ is chloro and R₂₀₁ is methyl (including CD₃).

In a particular group of compounds of the invention, the compounds have the structural formula Ih [a sub-definition of formula (I)] shown below, or a pharmaceutically acceptable salt, hydrate and/or solvate thereof:

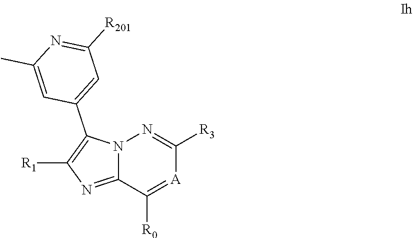

wherein A, R₀, R₁ and R₃ are each as defined hereinbefore and R₂₀₁ is selected from hydrogen, methyl (including CD₃), halo, trifluoromethyl, difluoromethyl, methoxy or acetyl.

In an embodiment of the compounds of formula Ih:
R₀ is as defined in either paragraph (1) or (2) above;
R₁ is as defined in any one of paragraphs (3) to (17) above;
R₃ is as defined in any one of paragraphs (31) to (48) above;
A is as defined in any one of paragraphs (49) to (54) above; and
R₂₀₁ is selected from methyl (including CD₃), methoxy or halo.

In another embodiment of the compounds of formula Ih:
R₀ is as defined in paragraph (1) above;
R₁ is as defined in paragraph (13) above;
R₃ is as defined in paragraph (34) above;
A is as defined in paragraph (50) above; and
R₂₀₁ is selected from methyl (including CD₃) or halo.

In another embodiment of the compounds of formula Ih:
R₀ is as defined in paragraph (1) above;
R₁ is as defined in paragraph (17) above;
R₃ is as defined in paragraph (37) or (38) above;
A is as defined in paragraph (52) or (53) above; and
R₂₀₁ is selected from methyl (including CD₃) or chloro.

In another embodiment of the compounds of formula Ih:
R₀ is as defined in paragraph (1) above;
R₁ is as defined in paragraph (17) above;
R₃ is as defined in paragraph (39) above;
A is as defined in paragraph (52) or (53) above; and
R₂₀₁ is selected from methyl (including CD₃) or chloro.

In a particular group of compounds of the invention, the compounds have the structural formula Ih [a sub-definition of formula (I)] shown below, or a pharmaceutically acceptable salt, hydrate and/or solvate thereof:

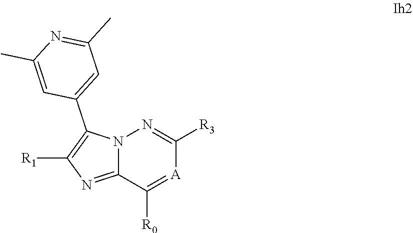

wherein A, R₀, R₁ and R₃ are each as defined hereinbefore.

In an embodiment of the compounds of formula Ih2:
R₀ is as defined in either paragraph (1) or (2) above;
R₁ is as defined in any one of paragraphs (3) to (17) above;
R₃ is as defined in any one of paragraphs (31) to (48) above; and
A is as defined in any one of paragraphs (49) to (54).

In another embodiment of the compounds of formula Ih2:
R₀ is as defined in paragraph (1) above;
R₁ is as defined in paragraph (13) above;
R₃ is as defined in paragraph (34) above; and
A is as defined in paragraph (50).

In another embodiment of the compounds of formula Ih2:
R₀ is as defined in paragraph (1) above;
R₁ is as defined in paragraph (17) above;
R₃ is as defined in paragraph (37) or (38) above; and
A is as defined in paragraph (52) or (53).

In another embodiment of the compounds of formula Ih2:
R₀ is as defined in paragraph (1) above;
R₁ is as defined in paragraph (17) above;
R₃ is as defined in paragraph (39) above; and
A is as defined in paragraph (52) or (53).

In a particular group of compounds of the invention, the compounds have the structural formula Ii [a sub-definition of formula (I)] shown below, or a pharmaceutically acceptable salt, hydrate and/or solvate thereof:

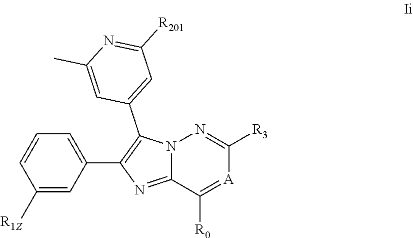

wherein A, R₀, R₃ and R_{1Z} are each as defined hereinbefore and R₂₀₁ is selected from hydrogen, methyl, halo, trifluoromethyl, difluoromethyl, methoxy or acetyl.

In an embodiment of the compounds of formula Ii:
R₀ is as defined in either paragraph (1) or (2) above;
R_{1z} is as defined in any one of paragraphs (3) to (11) above;
R₃ is as defined in any one of paragraphs (31) to (48) above;
A is as defined in any one of paragraphs (49) to (54) above; and
R₂₀₁ is selected from methyl (including CD₃), halo, trifluoromethyl, difluoromethyl, methoxy or acetyl.

In another embodiment of the compounds of formula Ii:
R₀ is as defined in paragraph (1) above;
R_{1z} is halo or cyano;
R₃ is as defined in paragraph (34) above;
A is as defined in paragraph (50) above; and
R₂₀₁ is selected from methyl (including CD₃), halo or methoxy.

In another embodiment of the compounds of formula Ii:
R₀ is as defined in paragraph (1) above;
R_{1z} is cyano;
R₃ is as defined in paragraph (37) or (38) above;
A is as defined in paragraph (52) or (53) above; and
R₂₀₁ is selected from methyl (including CD₃) or chloro.

In another embodiment of the compounds of formula Ii:
R₀ is as defined in paragraph (1) above;
R_{1z} is cyano;
R₃ is as defined in paragraph (39) above;
A is as defined in paragraph (52) or (53) above; and
R₂₀₁ is selected from methyl (including CD₃) or chloro.

In a particular group of compounds of the invention, the compounds have the structural formula Ii [a sub-definition of formula (I)] shown below, or a pharmaceutically acceptable salt, hydrate and/or solvate thereof:

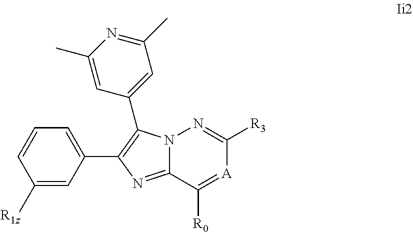

wherein A, R₀, R₃ and R_{1Z} are each as defined hereinbefore.

In an embodiment of the compounds of formula Ii2:
R₀ is as defined in either paragraph (1) or (2) above;
R_{1z} is as defined in any one of paragraphs (3) to (11) above;
R₃ is as defined in any one of paragraphs (31) to (38) above; and
A is as defined in any one of paragraphs (40) to (45).

In another embodiment of the compounds of formula Ii2:
R₀ is as defined in paragraph (1) above;
R_{1z} is halo or cyano;
R₃ is as defined in paragraph (34) above; and
A is as defined in paragraph (41).

In another embodiment of the compounds of formula Ii2:
R₀ is as defined in paragraph (1) above;
R_{1z} is cyano;
R₃ is as defined in paragraph (37) or (38) above; and
A is as defined in paragraph (44) or (45) above.

In a particular group of compounds of the invention, the compounds have the structural formula Ig [a sub-definition of formula (I)] shown below, or a pharmaceutically acceptable salt, hydrate and/or solvate thereof:

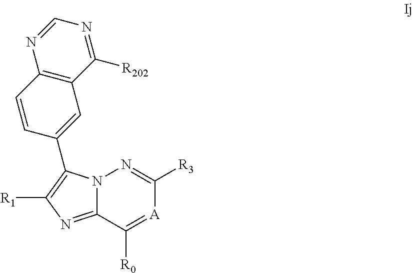

wherein A, R₀, R₁ and R₃ are each as defined hereinbefore; and R₂₀₀ and R₂₀₁ are each independently selected from hydrogen, methyl, halo, trifluoromethyl, difluoromethyl, methoxy or acetyl.

In an embodiment of the compounds of formula Ig:
R₀ is as defined in either paragraph (1) or (2) above;
R₁ is as defined in any one of paragraphs (3) to (17) above;
R₃ is as defined in any one of paragraphs (31) to (48) above;
A is as defined in any one of paragraphs (49) to (54) above; and
R₂₀₂ is selected from hydrogen, methyl (including CD₃) or halo.

In an embodiment of the compounds of formula Ig:
R₀ is as defined in either paragraph (1) or (2) above;
R₁ is as defined in any one of paragraphs (3) to (17) above;
R₃ is as defined in any one of paragraphs (31) to (39) above;
A is as defined in any one of paragraphs (49) to (54) above; and
R₂₀₂ is selected from hydrogen, methyl (including CD₃) or halo.

In another embodiment of the compounds of formula Ig:
R₀ is as defined in paragraph (1) above;
R₁ is as defined in paragraph (13) above;
R₂ is as defined in paragraph (25) above;
R₃ is as defined in paragraph (34) above;
A is as defined in paragraph (50) above; and
R₂₀₂ is selected selected from methyl (including CD₃) or chloro.

In another embodiment of the compounds of formula Ig:
R₀ is as defined in paragraph (1) above;
R₁ is as defined in paragraph (17) above;
R₂ is as defined in paragraph (30) above;
R₃ is as defined in paragraph (37) or (38) above;
A is as defined in paragraph (52) or (53) above; and
R₂₀₂ is methyl (including CD₃).

In another embodiment of the compounds of formula Ig:
R₀ is as defined in paragraph (1) above;
R₁ is as defined in paragraph (17) above;
R₂ is as defined in paragraph (30) above;
R₃ is as defined in paragraph (39) above;
A is as defined in paragraph (52) or (53) above; and
R₂₀₂ is methyl (including CD₃).

Particular compounds of the present invention include any of the compounds described in the example section of the present application, or a pharmaceutically acceptable salt or solvate thereof, and, in particular, any of the following:
3-Bromo-2-(3-cyanophenyl)-N-(2-hydroxy-2-methyl-propyl)imidazo[1,2-b]pyridazine-6-carboxamide;
3-(2-Acetyl-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-3-(2,6-dimethyl-4-pyridyl)-N-(2-hydroxy-2-methyl-propyl)imidazo[1,2-b]pyridazine-6-carboxamide;
3-(2-Chloro-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-N-(2-hydroxy-2-methyl-propyl)-3-[2-methyl-6-(trifluoromethyl)-4-pyridyl]imidazo[1,2-b]pyridazine-6-carboxamide;
2-(3-Cyanophenyl)-N-(2-hydroxy-2-methyl-propyl)-3-(2-methoxy-6-methyl-4-pyridyl)imidazo[1,2-b]pyridazine-6-carboxamide;
2-(3-Cyanophenyl)-3-[2-(difluoromethyl)-6-methyl-4-pyridyl]-N-[(1S)-2-hydroxy-1,2-dimethyl-propyl]imidazo[1,2-b]pyridazine-6-carboxamide;
2-(3-Cyanophenyl)-N-[(1S)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1R)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1S)-2-hydroxy-1,2-dimethyl-propyl]-3-(2-methoxy-6-methyl-4-pyridyl)imidazo[1,2-b]pyridazine-6-carboxamide;
3-(2-Chloro-6-methyl-4-pyridyl)-2-(3-cyanophenyl)-N-[(1S)-2-hydroxy-1,2-dimethyl-propyl]imidazo[1,2-b]pyridazine-6-carboxamide;
2-(3-Cyanophenyl)-3-(2,6-dimethyl-4-pyridyl)-N-[(1S)-2-hydroxy-1,2-dimethyl-propyl]imidazo[1,2-b]pyridazine-6-carboxamide;
3-[2,6-Bis(trideuteriomethyl)-4-pyridyl]-2-(3-cyanophenyl)-N-[(1S)-2-hydroxy-1,2-dimethyl-propyl]imidazo[1,2-b]pyridazine-6-carboxamide;
2-(3-cyanophenyl)-N-[(1S)-2-hydroxy-1,2-dimethyl-propyl]-3-(4-methylquinazolin-6-yl)imidazo[1,2-b]pyridazine-6-carboxamide;
2-(3-cyanophenyl)-N-[(1S)-2-hydroxy-1,2-dimethyl-propyl]-3-[2-(hydroxymethyl)-6-methyl-4-pyridyl]imidazo[1,2-b]pyridazine-6-carboxamide.

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid addition—salt of a compound of the invention which is sufficiently basic, for example, an acid addition—salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are nonsuperimposable mirror images of each other are termed “enantiomers”. When a compound has an asymmetric center-, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarized light and designated as dextrorotatory or -levorotatory- (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are wellknown in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For Example, H may be in any isotopic form, including 1H, 2H(D), and 3H (T); C may be in any isotopic form, including 12C, 13C, and 14C; and O may be in any isotopic form, including 16O and 18O; and the like. For example, a methyl group encompasses CH₃ and CD₃.

It is also to be understood that certain compounds of the formula (I) may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

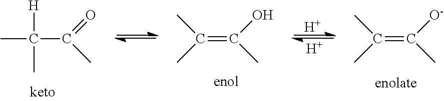

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular Examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a prodrug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula (I) as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988); f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C1-6alkyl esters such as methyl, ethyl and tert-butyl, C1-6alkoxymethyl esters such as methoxymethyl esters, C1-6alkanoyloxymethyl esters such as pivaloyloxymethyl esters, 3-phthalidyl esters, C3-8cycloalkylcarbonyloxy-C1-6alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C1-6alkoxycarbonyloxy-C1-6alkyl esters such as methoxycarbonyloxymethyl and 1-methoxycarbonyloxyethyl esters.

A suitable pharmaceutically acceptable pro-drug of a compound of the Formula (I) that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C1-10alkanoyl groups such as acetyl, benzoyl, phenylacetyl and substituted benzoyl and phenylacetyl groups, C1-10alkoxycarbonyl groups such as ethoxycarbonyl, N,N—(C1-6)2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C1-4alkylamine such as methylamine, a (C1-4alkyl)2amine such as dimethylamine, N-ethyl-N-methylamine or diethylamine, a C1-4alkoxy-C2-4alkylamine such as 2-methoxyethylamine, a phenyl-C1-4alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C1-10alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of the compounds of the invention are described in the Example section below.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of Example, a suitable protecting group for an amino or alkylamino group is, for example, an acyl group, for example an alkanoyl group such as acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladiumoncarbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladiumoncarbon.

A suitable protecting group for a carboxy group is, for example, an esterifying group, for example a methyl or an ethyl group which may be removed, for example, by hydrolysis with a base such as sodium hydroxide, or for example a tbutyl group which may be removed, for example, by treatment with an acid, for example an organic acid such as trifluoroacetic acid, or for example a benzyl group which may be removed, for example, by hydrogenation over a catalyst such as palladiumoncarbon.

Resins may also be used as a protecting group.

The methodology employed to synthesise a compound of formula (I) will vary depending on the nature of A, R₁, R₂ and R₃ and any substituent groups associated therewith. Suitable processes for their preparation are described further in the accompanying Examples.

Once a compound of formula (I) has been synthesised by any one of the processes defined herein, the processes may then further comprise one or more of the additional steps of:
(i) removing any protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An Example of (ii) above is when a compound of formula (I) is synthesised and then one or more of the groups of A, R₁, R₂ or R₃, may be further reacted to change the nature of the group and provide an alternative compound of formula (I).

The resultant compounds of formula (I) can be isolated and purified using techniques well known in the art.

Certain compounds of formula I defined herein may be prepared by:
(i) reacting a compound of formula IIa

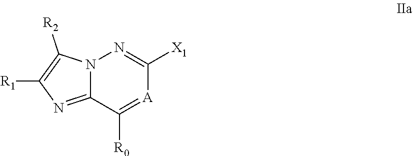

wherein A, R₀, R₁ and R₂ are each as defined hereinbefore, and X₁ is a suitable leaving group (e.g. bromo, chloro, iodo, —SMe, —S(O)Me or —S(O)₂Me);
with a group [R₃—X₂]—H, wherein X₂ is N, S or O and [R₃—X₂] together represent a group R₃ as defined hereinbefore that is linked through a X₂ atom;
and optionally thereafter, the process may further comprise one or more of the additional steps of:
(i) removing any protecting groups that may be present;
(ii) converting the compound formula (I) into another compound of formula (I) (e.g. converting a R₃ substituent into another R₃ substituent group as defined herein);
(iii) forming a pharmaceutically acceptable salt, hydrate or solvate of the compound of formula I; and/or
(iv) forming a prodrug of the compound of formula I.

For the avoidance of any doubt, the X₂ atom is a heteroatom present in a group R₃, i.e. [R₃—X₂] is a R₃ group comprising a X₂ heteroatom.

It will be appreciated that, in the above reaction, if a compound of formula II is reacted with a [R₃—X₂]—H group, the X₁ group is displaced along with the H atom of the [R₃—X₂]—H group and the R₃ substituent group is bound to the compound of formula II via the X₂ atom.

A person skilled in the art, will be able to readily select suitable reaction conditions for the reaction between a compound of formula II and a [R₃—X₂]—H group. Examples of suitable reaction conditions are described in the accompanying example section herein.

Compounds of formula II can be prepared by suitable techniques known in the art, as will be evident from the accompany example section. Particular examples of the preparation of compounds of formula II are described in the accompanying example section herein.

Compounds of formula I may also be prepared by Suzuki-Miyaura or Stille coupling reactions. For example, certain compounds of formula I defined herein may also be prepared by:
(i) reacting a compound of formula III:

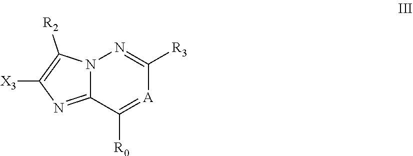

wherein A, R₀, R₂ and R₃ are each as defined hereinbefore and X₃ is a halo atom (e.g. bromo, chloro or iodo);
with a group of the formula:

R₁-M
wherein M is a coupling reagent (e.g. a boron coupling agent or a tin coupling agent as defined herein) and R₁ is as defined hereinbefore; or
(ii) reacting a compound of formula IV:

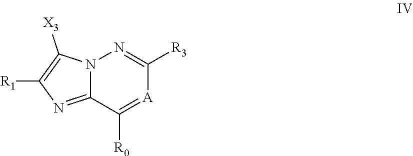

wherein A, R₀, R₁ and R₃ are each as defined hereinbefore and X₃ is a halo atom (e.g. bromo, chloro or iodo);
with a group of the formula:

R₂-M
wherein M is a coupling reagent (e.g. a boron coupling agent or a tin coupling agent as defined herein) and R₂ is as defined hereinbefore; or
(iii) reacting a compound of formula V:

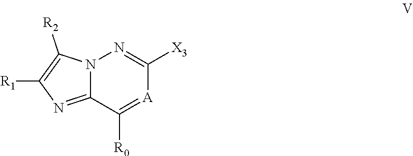

wherein A, R₀, R₁ and R₂ are each as defined hereinbefore and X₃ is a halo atom (e.g. bromo, chloro or iodo);
with a group of the formula:

R₃-M
wherein M is a coupling reagent (e.g. a boron coupling agent or a tin coupling agent as defined herein) and R₃ is as defined hereinbefore; or
and optionally thereafter, the process may further comprise one or more of the additional steps of:
(i) removing any protecting groups that may be present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

The group M may be a suitable boron coupling reagent known in the art for Suzuki-Miyaura coupling reactions. Examples of suitable boron agents include: boronic acid, boronic esters (e.g. catechol boronic acid ester, pinacol boronic acid ester, triisopropyl boronate, MIDA boronate, cyclctriol boronate), boranes (e.g. 9-BBN borane), organotrifluoroborate or boronamides (e.g. 1,8-diaminonaphthyl boronamide). Particular examples are —B(OH)₂ or —B(OCH₃)₂.

Suzuki-Miyaura coupling reactions are well known and a person skilled in the art will be able to readily select suitable reaction conditions for this reaction.

In Stille coupling reactions, M is a tin coupling agent, suitably a tin coupling agent of the formula —Sn[(1-6C)alkyl]₃, for example —Sn(butyl)₃.

Stille coupling reactions are well known and a person skilled in the art will be ale to readily select suitable reaction conditions for this reaction. Such reactions are typically carried out in the presence of a palladium catalyst.

### Biological Activity

The biological assays described in the example section (Biological Examples 1 to 3) may be used to measure the pharmacological effects of the compounds of the present invention.

Although the pharmacological properties of the compounds of formula I vary with structural change, as expected, the compounds of the invention were found to be active in the assays described in Biological Examples 1, 2 and 3.

In general, in terms of adenosine A2a antagonism, the compounds of the invention demonstrate an IC₅₀ of 1 μM or less in the assay described in Biological Example 1, with preferred compounds of the invention demonstrating an IC₅₀ of 200 nM or less and the most preferred compounds of the invention demonstrating an IC₅₀ of 50 nM or less.

Suitably the IC₅₀ at the adenosine A1, A2b or A3 receptors of the compounds of the invention in the assay described in Biological Example 1 is at least two-fold higher than the IC₅₀ at the adenosine A2a receptor, and more suitably it is at least 5-fold higher, and even more suitably it is at least 10-fold higher.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For Example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as antagonists of adenosine A2 receptors, especially adenosine A2a receptors.

According to a further aspect of the present invention, there is provided a method of antagonising adenosine A2a receptors in vitro or in vivo, said method comprising contacting a cell with an effective amount of a compound or a pharmaceutically acceptable salt, hydrate or solvate thereof as defined herein.

According to a further aspect of the present invention, there is provided a method of selectively antagonising adenosine A2a receptors in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder in which adenosine A2a receptor activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an adenosine A2a antagonist. In an embodiment, the compounds of the invention are selective adenosine A2a antagonists. In an alternative embodiment, certain compounds of the invention are selective adenosine A2a and adenosine A2b antagonists.

According to a further aspect of the present invention, there is provided a compound, or a pharmaceutically acceptable salt, hydrate or solvate thereof, as defined herein for use in the treatment of a disease or disorder in which adenosine A2a is implicated.

According to a further aspect of the present invention, there is provided the use of a compound, or a pharmaceutically acceptable salt, hydrate or solvate thereof, as defined herein in the manufacture of a medicament for the treatment of a proliferative condition.

Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for use as an adenosine A2a antagonist.

According to a further aspect of the present invention, there is provided a use of a compound, or a pharmaceutically acceptable salt, hydrate or solvate thereof, as defined herein in the manufacture of a medicament for the treatment of a disease or disorder in which adenosine A2a receptor activity is implicated.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compounds of the present invention have particular application in the treatment of human cancers (by virtue of their adenosine A2a antagonist activity).

More specifically, there is provided a compound of general formula (I) for use in the treatment of cancer, particularly solid tumours, for example non-small cell lung cancer, head and neck squamous cancer and urothelial cancer.

There is also provided the use of a compound of general formula (I) in the manufacture of a medicament for the treatment of cancer, particularly solid tumours, for example non-small cell lung cancer, head and neck squamous cancer and urothelial cancer.

The invention further provides a method for the treatment of cancer, particularly solid tumours, for example non-small cell lung cancer, head and neck squamous cancer and urothelial cancer, the method comprising administering to a patient in need of such treatment an effective amount of a compound of general formula (I).

The patient to be treated is suitably a mammal and more suitably a human.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of formula I are useful for the treatment and/or prophylaxis of proliferative disorders, such as, for example, cancer. A compound of formula I defined herein may be used in combination with one or more additional antiproliferative/anticancer therapies, such as, for example, chemotherapy with one or more additional antiproliferative/anticancer agents, radiotherapy and/or conventional surgery.

An additional antiproliferative/anticancer agent may be included in the pharmaceutical composition with a compound of formula (I) as defined herein or, alternatively, it may be administered separately, either at the same time as the compound of formula (I) or at an earlier or later time.

Therefore, in a further aspect of the present invention there is provided a product comprising a compound of general formula (I) and an additional agent useful in the treatment or prevention of cancer as a combined preparation for simultaneous, sequential or separate use in the treatment of cancer.

The present invention also provides a compound of general formula (I) in combination with one or more additional antiproliferative/anticancer agents for use in the treatment of cancer as a combined preparation for simultaneous, sequential or separate use in the treatment of treatment of cancer.

In particular, the combination therapy defined herein is suitable for the treatment of solid tumours for example non-small cell lung cancer, head and neck squamous cancer and urothelial cancer.

Suitable additional antiproliferative/anti-cancer agents that may be used in combination with a compound of formula I defined herein [either separately or as part of a combined pharmaceutical composition or a combined preparation with the compounds of general formula (I)] include:
1) other forms of cancer immunotherapy and anti-cancer chemotherapeutic agents;
2) adenosine pathway modulators, including, but not limited to A2b antagonists, CD73 inhibitors and CD39 inhibitors; (suitably A2b antagonists);
3) anti-PD-1 and PDL-1 antibodies including, but not limited to, pembrolizumab, nivolumab, durvalumab, avelumab and atezolizumab; and
4) anti-CTLA4 antibodies including, but not limited to, ipilimumab.

The compounds of of formula I defined herein are particularly suited to use in combination with anti-PD-1 and PDL-1 antibodies including, but not limited to, pembrolizumab, nivolumab, durvalumab, avelumab and atezolizumab.

Suitably, the anti-PD1 antibody is one of the antibodies disclosed in U.S. Publication No. 2019/0225689 or U.S. Publication No. 2017/0121409 (incorporated herein by reference in their entireties), such as cetrelimab. Cetrelimab (JNJ-63723283, CET) is a fully human immunoglobulin (Ig) G4 kappa monoclonal antibody that binds to programmed death receptor-1 (PD-1) with high affinity and specificity. Cetrelimab has shown activity in solid tumors. Rutkowski P, et al. Journal of Clinical Oncology. 2019; 37(8):31.

The compounds of of formula I defined herein are particularly suited to use in combination with adenosine pathway modulators, including, but not limited to A2b antagonists, CD73 inhibitors and CD39 inhibitors.

The A2a antagonists of general formula (I) can also be used in combination with cell-based immunotherapy and cancer vaccines that include, but are not limited to CAR-T cell therapy.

Examples of the additional antiproliferative/anticancer chemotherapeutic agents include, but are not limited to, any one or more of the following:
MEK (e.g. MEK1, MEK2, or MEK1 and MEK2) inhibitors (e.g. XL518, CI-1040, PD035901, selumetinib/AZD6244, GSK1 120212/trametinib, GDC-0973, ARRY-162, ARRY-300, AZD8330, PD0325901, U0126, PD98059, TAK-733, PD3 18088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, arboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-I, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin Ill derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fiudarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatinA; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzyl guanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-basedimmune modulator; protein kinase C inhibitor; protein kinase C inhibitors, microalgal; proteintyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAPinhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stemcell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin 2 (including recombinant interleukin 2, or rIL.sub.2), interferon alfa-2a; interferon alfa-2b;
interferon alfa-nI; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-3 10705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin AI (i.e. BTO-956 and DF E), Fijianolide B, Laulimalide, Narcosine (also known as NSC-5366), Nascapine, Hemiasterlin, Vanadocene acetylacetonate, Monsatrol, Inanocine (i.e. NSC-698666), Eleutherobins (such as Desmethyleleutherobin, Desaetyleleutherobin, Isoeleutherobin A, and ZEleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-F£R2, anti-CD52, anti-ULA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to In, 0Y, or I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), japatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

As indicated above, the combination therapy of the present invention may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and one or more additional antiproliferative/anticancer agents.

According to this aspect of the invention there is also provided a combination for use in the treatment of a proliferative condition, such as cancer (for example a cancer involving a solid tumour), comprising a compound of the invention as defined hereinbefore, or a pharmaceutically acceptable salt, hydrate or solvate thereof, and one or more additional antiproliferative/anticancer agents selected from those listed above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### EXAMPLE SECTION

### General Conditions:

Mass spectra were run on LC-MS systems using electrospray ionization. These were run using either a Waters Acquity H-Class UPLC with PDA and QDa mass detection, an Acquity UPLC (binary pump/PDA detector)+ZQ Mass Spectrometer or Acquity i-Class (quaternary pump/PDA detector)+Quattro Micro Mass Spectrometer, a Waters Acquity uPLC system with Waters PDA and ELS detectors or a Shimadzu LCMS-2010EV system. [M+H]+refers to mono-isotopic molecular weights.

NMR spectra were run on either a Bruker Ultrashield 500 MHz NMR spectrometer, a Bruker Avance III HD 400 MHz NMR spectrometer, a Bruker Avance DPX 300 MHz NMR spectrometer a Bruker Avance III HD 500 MHz or a Bruker Avance III HD 250 MHz. Spectra were recorded at 298K and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

### Abbreviation

app apparent
br broad
d doublet
dd doublet of doublets
DCM dichloromethane
DIPEA diisopropylethylamine
DMF N,N-dimethylformamide
EtOAc ethyl acetate
HPLC high pressure liquid chromatography
IMS industrial methylated spirit
LC-MS liquid chromatography and mass spectrometry
MeOH MeOH
MeCN acetonitrile
MS mass spectrometry
m multiplet
mins minute(s)
mL milliliter(s)
m/z mass to charge ratio
NMR nuclear magnetic resonance
ppm parts per million
Rt retention time
s singlet
t triplet
TBAF tetra-n-butylammonium fluoride
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

If not indicated otherwise, the analytical HPLC conditions are as follows:

### Method 2B

| | |
|---|---|
| Column: | Acquity UPLC BEH C18 2.1 × 50 mm, 1.7 μm |
| Column Temp: | 50° C. |
| Eluents: | A: H₂O, 0.1% formic acid B: MeCN |
| Flow Rate: | 0.8 mL/min |
| Gradient: | 0.0-1.8 min 2-98% B, |
| | 1.8-2.1 min 98% B, |
| | 2.1-2.5 min 98% A |

### Method 3A

| | |
|---|---|
| Column: | Acquity UPLC CSH C18 2.1 × 50 mm, 1.7 μm |
| Column Temp: | 50° C. |
| Eluents: | A: H₂O, B: MeCN, 0.1% formic acid |
| Flow Rate: | 1 mL/min |
| Gradient: | 0.2-2.5 min 2-98% B, 2.5-3.0 min 98% B |

### Method 3B

| | |
|---|---|
| Column: | Acquity UPLC BEH C18 2.1 × 50 mm, 1.7 μm |
| Column Temp: | 50° C. |
| Eluents: | A: H₂O, B: MeCN, 0.1% ammonia |
| Flow Rate: | 1 mL/min |
| Gradient: | 0.2-2.5 min 2-98% B, 2.5-3.0 min 98% B |

### Method 3.5B

| | |
|---|---|
| Column: | Acquity UPLC BEH C18 2.1 × 50 mm 1.7 μm |
| Column Temp: | 40° C. |
| Eluents: A: | H₂O, 0.1% ammonia B: MeCN |
| Flow Rate: | 0.6 mL/min |
| Gradient: | 0.2-2.5 min 2-98% B, 2.5-3.3 min 98% B |

### Method 5A

| | |
|---|---|
| Column: | YMC-Triart C18 2 × 50 mm, 5 μm. |
| Flow rate: | 0.8 mL/min. |
| Eluents: | A: H₂O, B: MeCN, C: 50% H₂O/50% |
| | MeCN + 1.0% formic acid |
| Gradient: | 0.0-4.0 min 0-95% B, |
| | 5% C; 4.0-4.4 min 95% B, |
| | 5% C; 4.4-4.5 min 95% A, 5% B |

### Method 5B

| | |
|---|---|
| Column: | YMC-Triart C18 2 × 50 mm, 5 μm. |
| Flow rate: | 0.8 mL/min. |
| Eluents: | A: H₂O, B: MeCN, C: 50% H₂O / 50% MeCN + |
| | 1.0% ammonia (aq.) |
| Gradient: | 0.0-4.0 min 0-95% B, 5% C; 4.0-4.4 min 95% B, |
| | 5% C; 4.4-4.5 min 95% A, 5% B |

### Method 8B

| | |
|---|---|
| Column: | Acquity UPLC BEH C18 2.1 × 100 mm, 1.7 μm |
| Column Temp: | 50° C. |
| Eluents: | A: H₂O, B: MeCN, 0.1% ammonia |
| Flow Rate: | 0.6 mL/min |
| Gradient: | 0.5-6.5 min 2-98% B 6.5-7.5 min 98% B |

### Example 1

### 3-Bromo-2-(3-cyanophenyl)-N-(2-hydroxy-2-methyl-propyl)imidazo[1,2-b]pyridazine-6-carboxamide

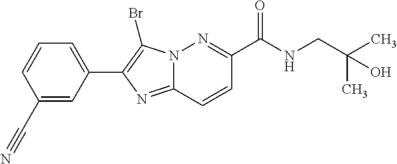 [SMILES: CC(C)(O)CNC(=O)c1ccc2nc(-c3cccc(C#N)c3)c(Br)n2n1]

T3P® (50% in DMF) (808 μL, 1.15 mmol) was added dropwise to a stirred solution of 3-bromo-2-(3-cyanophenyl)imidazo[1,2-b]pyridazine-6-carboxylic acid (Intermediate B) (197 mg, 0.57 mmol), 1-amino-2-methyl-propan-2-ol (80 μL, 0.86 mmol) and DIPEA (700 μL, 4.02 mmol) in anhydrous DMF (3 mL) under an atmosphere of nitrogen atmosphere and the mixture was stirred at room temperature for 70 mins. The resulting mixture was added to stirring water (20 mL), the suspension stirred for 10 mins and then filtered. The solids were washed with water (2×20 mL) and vacuum dried to afford the title compound as a cream solid.

LC-MS (Method 8B): Rt 3.92 mins; MS m/z 414.1/416.1=[M+H]+

¹H NMR (500 MHz, DMSO) δ 8.51 (s, 1H), 8.49 (d, J=8.5 Hz, 1H), 8.37 (d, J=9.4 Hz, 1H), 8.27 (t, J=6.2 Hz, 1H), 7.96 (d, J=7.7 Hz, 1H), 7.86 (d, J=9.4 Hz, 1H), 7.80 (apr t, J=7.8 Hz, 1H), 4.76 (s, 1H), 3.36 (d, J=6.2 Hz, 2H), 1.16 (s, 6H).

### Example 2

### 3-(2-Acetyl-6-methyl-4-pyridyl)-2-(3-cyanophenyl)-N-(2-hydroxy-2-methyl-propyl)imidazo[1,2-b]pyridazine-6-carboxamide

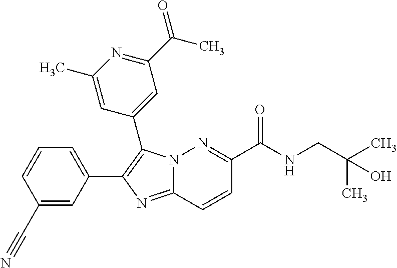 [SMILES: CC(=O)c1cc(-c2c(-c3cccc(C#N)c3)nc3ccc(C(=O)NCC(C)(C)O)nn23)cc(C)n1]

A solution of K₂CO₃ (75 mg, 0.54 mmol) in water (0.8 mL) was added to a solution of 3-bromo-2-(3-cyanophenyl)-N-(2-hydroxy-2-methyl-propyl)imidazo[1,2-b]pyridazine-6-carboxamide (Example 1/Intermediate C)(75 mg, 0.18 mmol) in 1,4-dioxane (3.2 mL) and the resulting mixture was de-oxygenanted via nitrogen sparging for 10 mins. Xphos Pd G3 (15 mg, 0.02 mmol) and 1-[6-methyl-4-(4,4,5,5-tetramethyl-1,3,2-dioxaborolan-2-yl)-2-pyridyl]ethanone (71 mg, 0.27 mmol) were added and the reaction mixture was stirred under an atmosphere of nitrogen at 50° C. for 1 h. After cooling to room temperature, the mixture was diluted with water (10 mL) and extracted with EtOAc (3×10 mL). The combined organic layers were dried over Na₂SO₄ and concentrated in vacuo. Purification of the crude material by chromatography on silica eluting with a gradient of 0 to 4% MeOH in DCM followed by further purification by C18 reverse phase chromatography eluting with 5 to 25% MeCN in water (+0.1% ammonium hydroxide modifier) afforded the product as a glassy syrup. Trituration with diethyl ether (10 mL) afforded the title compound as a yellow solid.

LC-MS (Method 8B): Rt 4.13 mins; MS m/z 469.3=[M+H]+

1H NMR (500 MHz, DMSO) δ 8.43 (d, J=9.4 Hz, 1H), 8.12-8.03 (m, 3H), 7.91 (dd, J=7.9, 1.7 Hz, 2H), 7.88-7.82 (m, 2H), 7.63 (t, J=7.8 Hz, 1H), 4.64 (s, 1H), 3.27 (d, J=5.9 Hz, 2H), 2.66 (s, 3H), 2.64 (s, 3H), 1.12 (s, 6H).

The compounds of the following tabulated Examples (Table Ex2) were prepared analogously to Example 2 according to the following scheme from the appropriate Intermediate and commercially available boronate ester.

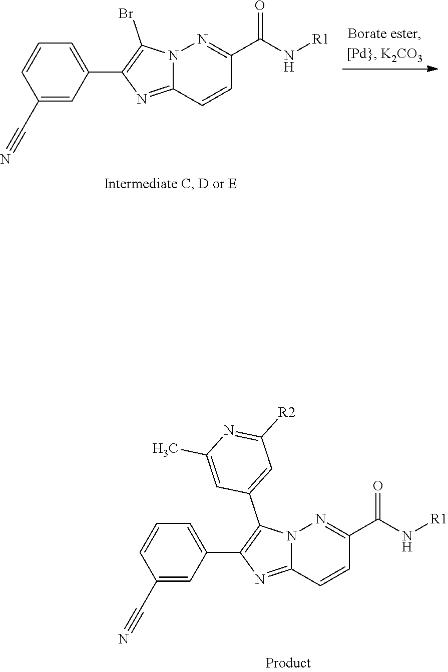

TABLE Ex2

| Ex. | Product | Retention Time, [M + H]+, ¹H NMR |
|---|---|---|
| 2.1 | 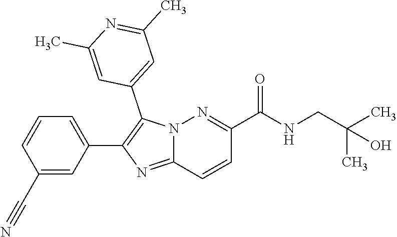 [SMILES: Cc1cc(-c2c(-c3cccc(C#N)c3)nc3ccc(C(=O)NCC(C)(C)O)nn23)cc(C)n1] | LC-MS (Method 8B): Rt 3.87 mins; MS m/z 441.3 = [M + H]+ ¹H NMR (500 MHz, DMSO-d6) δ 8.41 (d, J = 9.5 Hz, 1H), 8.09 (d, J = 1.6 Hz, 1H), 8.02 (t, J = 5.9 Hz, 1H), 7.93 (dd, J = 8.0, 1.7 Hz, 1H), 7.92-7.88 (m, 1H), 7.85 (d, J = 9.4 Hz, 1H), 7.65 (t, J = 7.8 Hz, 1H), 7.36 (s, 2H), 4.67 (s, 1H), 3.28 (d, J = 5.9 Hz, 2H), 2.47 (s, 6H), 1.15 (s, 6H). |
| | 2-(3-Cyanophenyl)-3-(2,6-dimethyl- | |
| | 4-pyridyl)-N-(2-hydroxy-2-methyl- | |
| | propyl)imidazo[1,2-b]pyridazine-6- | |
| | carboxamide | |
| | Prepared from Intermediate C | |
| 2.2 | 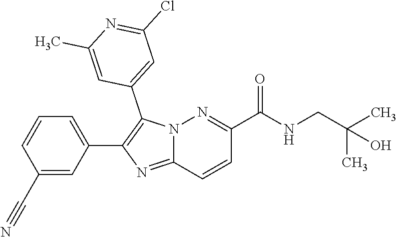 [SMILES: Cc1cc(-c2c(-c3cccc(C#N)c3)nc3ccc(C(=O)NCC(C)(C)O)nn23)cc(Cl)n1] | LC-MS (Method 8B): Rt 4.13 mins; MS m/z 461.2 = [M + H]+ ¹H NMR (500 MHz, DMSO-d6) δ 8.44 (d, J = 9.4 Hz, 1H), 8.17-8.10 (m, 2H), 7.97-7.90 (m, 2H), 7.87 (d, J = 9.4 Hz, 1H), 7.70-7.64 (m, 2H), 7.55 (s, 1H), 4.65 (s, 1H), 3.30-3.28 (m, 2H), 2.52- 2.48 (m, 3H), 1.15 (s, 6H) |
| | 3-(2-Chloro-6-methyl-4-pyridyl)-2-(3- | |
| | cyanophenyl)-N-(2-hydroxy-2- | |
| | methyl-propyl)imidazo[1,2- | |
| | b]pyridazine-6-carboxamide | |
| | Prepared from Intermediate C | |
| 2.3 | 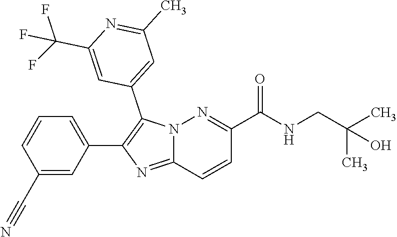 [SMILES: Cc1cc(-c2c(-c3cccc(C#N)c3)nc3ccc(C(=O)NCC(C)(C)O)nn23)cc(C(F)(F)F)n1] | LC-MS (Method 8B): Rt 4.42 mins; MS m/z 495.2 = [M + H]+ ¹H NMR (500 MHz, DMSO-d6) δ 8.45 (d, J = 9.4 Hz, 1H), 8.16 (t, J = 6.1 Hz, 1H), 8.10 (t, J = 1.5 Hz, 1H), 8.05 (s, 1H), 7.93 (tt, J = 7.9, 1.5 Hz, 2H), 7.88 (d, J = 9.4 Hz, 1H), 7.81 (s, 1H), 7.66 (t, J = 7.9 Hz, 1H), 4.62 (s, 1H), 3.28 (d, J = 6.1 Hz, 2H), 2.63 (s, 3H), 1.13 (s, 6H). |
| | 2-(3-Cyanophenyl)-N-(2-hydroxy-2- | |
| | methyl-propyl)-3-[2-methyl-6- | |
| | (trifluoromethyl)-4-pyridyl] | |
| | imidazo[1,2-b]pyridazine-6- | |
| | carboxamide | |
| | Prepared from Intermediate C | |
| 2.4 | 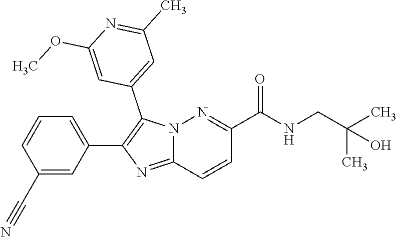 [SMILES: COc1cc(-c2c(-c3cccc(C#N)c3)nc3ccc(C(=O)NCC(C)(C)O)nn23)cc(C)n1] | LC-MS (Method 8B): Rt 4.25 mins; MS m/z 457.3 = [M + H]+ ¹H NMR (500 MHz, DMSO-d₆) δ 8.40 (d, J = 9.4 Hz, 1H), 8.09 (t, J = 1.7 Hz, 1H), 8.02 (t, J = 6.0 Hz, 1H), 7.92 (d, J = 7.7 Hz, 1H), 7.89 (d, J = 7.9 Hz, 1H), 7.83 (d, J = 9.4 Hz, 1H), 7.65 (t, J = 7.8 Hz, 1H), 7.21 (s, 1H), 6.82 (s, 1H), 4.64 (s, 1H), 3.86 (s, 3H), 3.28 (d, J = 6.0 Hz, 2H), 2.45 (s 3H), 1.13 (s, 6H). |
| | 2-(3-Cyanophenyl)-N-(2-hydroxy-2- | |
| | methyl-propyl)-3-(2-methoxy-6- | |
| | methyl-4-pyridyl)imidazo[1,2- | |
| | b]pyridazine-6-carboxamide | |
| | Prepared from Intermediate C | |
| 2.5 | 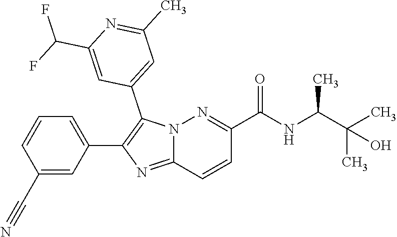 [SMILES: Cc1cc(-c2c(-c3cccc(C#N)c3)nc3ccc(C(=O)N[C@@H](C)C(C)(C)O)nn23)cc(C(F)F)n1] | LC-MS (Method 8B): Rt 4.29 mins; MS m/z 491.3 = [M + H]+ ¹H NMR (500 MHz, DMSO-d₆) δ 8.43 (d, J = 9.4 Hz, 1H), 8.09 (t, J = 1.7 Hz, 1H), 7.97-7.84 (m, 5H), 7.69-7.62 (m, 2H), 6.92 (t, J = 54.9 Hz, 1H), 4.63 (s, 1H), 3.94-3.84 (m, 1H), 2.60 (s, 3H), 1.17-1.08 (m, 9H). |
| | 2-(3-Cyanophenyl)-3-[2- | |
| | (difluoromethyl)-6-methyl-4-pyridyl]- | |
| | N-[(1S)-2-hydroxy-1,2-dimethyl- | |
| | propyl]imidazo[1,2-b]pyridazine-6- | |
| | carboxamide | |
| | Prepared from Intermediate D | |
| 2.6 | 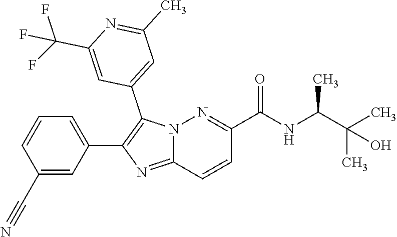 [SMILES: Cc1cc(-c2c(-c3cccc(C#N)c3)nc3ccc(C(=O)N[C@@H](C)C(C)(C)O)nn23)cc(C(F)(F)F)n1] | LC-MS (Method 8B): Rt 4.58 mins; MS m/z 509.2 = [M + H]+ ¹H NMR (500 MHz, DMSO-d6) δ 8.45 (d, J = 9.4 Hz, 1H), 8.11 (s, 1H), 8.01 (s, 1H), 7.97-7.90 (m, 3H), 7.90- 7.85 (m, 2H), 7.67 (t, J = 7.8 Hz, 1H), 4.59 (s, 1H), 3.95-3.87 (m, 1H), 2.62 (s, 3H), 1.20-1.05 (m, 9H). |
| | 2-(3-Cyanophenyl)-N-[(1S)-2- | |
| | hydroxy-1,2-dimethyl-propyl]-3-[2- | |
| | methyl-6-(trifluoromethyl)-4- | |
| | pyridyl]imidazo[1,2-b]pyridazine-6- | |
| | carboxamide | |
| | Prepared from Intermediate D | |
| 2.7 | 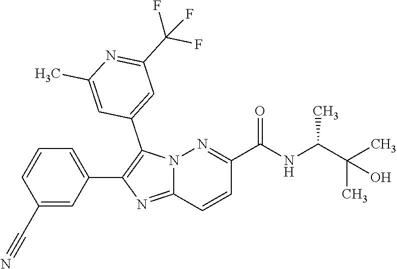 [SMILES: Cc1cc(-c2c(-c3cccc(C#N)c3)nc3ccc(C(=O)N[C@H](C)C(C)(C)O)nn23)cc(C(F)(F)F)n1] | LC-MS (Method 8B): Rt 4.58 mins; MS m/z 509.2 = [M + H]+ ¹H NMR (500 MHz, DMSO) δ 8.44 (d, J = 9.4 Hz, 1H), 8.11 (br s, 1H), 8.01 (br s, 1H), 7.98-7.90 (m, 3H), 7.89-7.85 (m, 2H), 7.67 (apr t, J = 7.8 Hz, 1H), 4.60 (s, 1H), 3.95-3.88 (m, 1H), 2.62 (s, 3H), 1.15 (s, 3H), 1.13 (d, J = 6.8 Hz, 3H), 1.10 (s, 3H). |
| | 2-(3-Cyanophenyl)-N-[(1R)-2- | |
| | hydroxy-1,2-dimethyl-propyl]-3-[2- | |
| | methyl-6-(trifluoromethyl)-4- | |
| | pyridyl]imidazo[1,2-b]pyridazine-6- | |
| | carboxamide | |
| | Prepared from Intermediate E | |
| 2.8 | 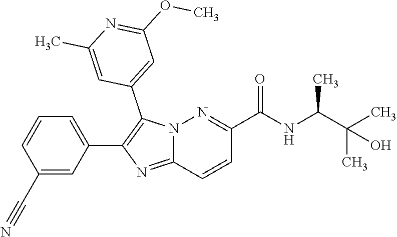 [SMILES: COc1cc(-c2c(-c3cccc(C#N)c3)nc3ccc(C(=O)N[C@@H](C)C(C)(C)O)nn23)cc(C)n1] | LC-MS (Method 8B): Rt 4.43 mins; MS m/z 471.3 = [M + H]+ ¹H NMR (500 MHz, DMSO-d6) δ 8.40 (d, J = 9.4 Hz, 1H), 8.10 (t, J = 1.5 Hz, 1H), 7.93 (dt, J = 7.8, 1.5 Hz, 1H), 7.89 (dt, J = 7.8, 1.5 Hz, 1H), 7.85 (d, J = 9.1 Hz, 1H), 7.83 (d, J = 9.4 Hz, 1H), 7.65 (t, J = 7.8 Hz, 1H), 7.24 (s, 1H), 6.79 (s, 1H), 4.63 (s, 1H), 3.92-3.87 (m, 1H), 3.86 (s, 3H), 2.46 (s, 3H), 1.19-1.10 (m, 9H). |
| | 2-(3-Cyanophenyl)-N-[(1S)-2- | |
| | hydroxy-1,2-dimethyl-propyl]-3-(2- | |
| | methoxy-6-methyl-4- | |
| | pyridyl)imidazo[1,2-b]pyridazine-6- | |
| | carboxamide | |
| | Prepared from Intermediate D | |
| 2.9 | 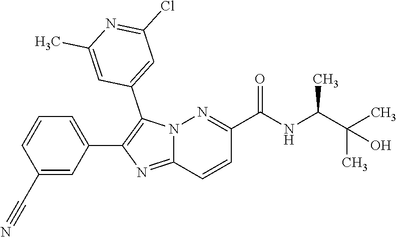 [SMILES: Cc1cc(-c2c(-c3cccc(C#N)c3)nc3ccc(C(=O)N[C@@H](C)C(C)(C)O)nn23)cc(Cl)n1] | LC-MS (Method 8B): Rt 4.35 mins; MS m/z 475.3 = [M + H]+ ¹H NMR (500 MHz, Methanol-d₄) δ 8.30 (d, J = 9.4 Hz, 1H), 8.09 (s, 1H), 8.00 (d, J = 9.4 Hz, 1H), 7.91 (dt, J = 7.9, 1.5 Hz, 1H), 7.82 (d, J = 7.9 Hz, 1H), 7.63 (t, J = 7.9 Hz, 1H), 7.59 (s, 1H), 7.54 (s, 1H), 4.06 (q, J = 6.8 Hz, 1H), 2.56 (s, 3H), 1.35-1.17 (m, 9H). 2 x exchangeable protons not observed. |
| | 3-(2-Chloro-6-methyl-4-pyridyl)-2-(3- | |
| | cyanophenyl)-N-[(1S)-2-hydroxy-1,2- | |
| | dimethyl-propyl]imidazo[1,2- | |
| | b]pyridazine-6-carboxamide | |
| | Prepared from Intermediate D | |

### Example 3

### 2-(3-Cyanophenyl)-3-(2,6-dimethyl-4-pyridyl)-N-[(1S)-2-hydroxy-1,2-dimethyl-propyl]imidazo[1,2-b]pyridazine-6-carboxamide

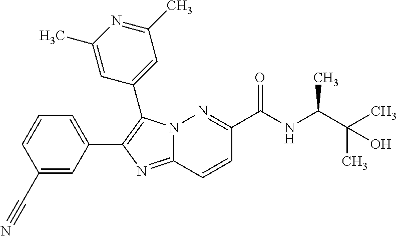 [SMILES: Cc1cc(-c2c(-c3cccc(C#N)c3)nc3ccc(C(=O)N[C@@H](C)C(C)(C)O)nn23)cc(C)n1]

To a degassed solution of 3-bromo-2-(3-cyanophenyl)-N-[(1S)-2-hydroxy-1,2-dimethyl-propyl]imidazo[1,2-b]pyridazine-6-carboxamide (Intermediate D)(75 mg, 0.18 mmol), 2,6-dimethyl-4-(4,4,5,5-tetramethyl-1,3,2-dioxaborolan-2-yl)pyridine (82 mg, 0.35 mmol) and K₂CO₃ (97 mg, 0.7 mmol) in 1,4-dioxane (2 mL) and water (0.5 mL) was added Pd(t-Bu₃P)₂ (9 mg, 0.02 mmol) and the mixture stirred at 50° C. for 1 h. The resulting mixture was allowed to cool to room temperature and concentrated in vacuo. Purification by chromatography on silica eluting with a gradient of 0 to 4% MeOH in DCM afforded a solid. The solid was triturated in diethyl ether (2 mL), filtered washing with diethyl ether (2×1 mL) and dried under vacuum to afford the title compound as a yellow solid.

LC-MS (Method 8B): Rt 4.05 mins; MS m/z 455.3=[M+H]+

¹H NMR (500 MHz, DMSO-d₆) δ 8.40 (d, J=9.4 Hz, 1H), 8.09 (s, 1H), 7.93 (d, J=8.0 Hz, 1H), 7.91-7.85 (m, 2H), 7.83 (d, J=9.4 Hz, 1H), 7.65 (t, J=7.8 Hz, 1H), 7.35 (s, 2H), 4.67 (s, 1H), 3.91-3.84 (m, 1H), 2.47 (s, 6H), 1.18-1.10 (m, 9H).

### Example 4

### 3-[2,6-Bis(trideuteriomethyl)-4-pyridyl]-2-(3-cyanophenyl)-N-[(1S)-2-hydroxy-1,2-dimethyl-propyl]imidazo[1,2-b]pyridazine-6-carboxamide

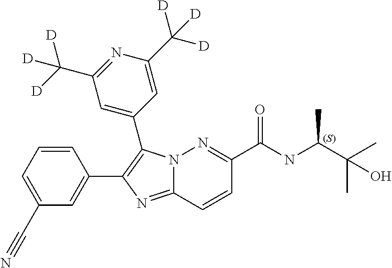

A solution of K₂CO₃ (30 mg, 2.24 mmol) in water (1 mL) was added to a solution of 3-bromo-2-(3-cyanophenyl)-N-[(1S)-2-hydroxy-1,2-dimethyl-propyl]imidazo[1,2-b]pyridazine-6-carboxamide (Intermediate D)(240 mg, 0.56 mmol) and crude 4-(4,4,5,5-tetramethyl-1,3,2-dioxaborolan-2-yl)-2,6-bis(trideuteriomethyl)pyridine (Intermediate F)(268 mg, 1.12 mmol) in 1,4-dioxane (4 mL) and the resulting mixture de-oxygenated via nitrogen sparging for 10 mins. Pd(^{t}Bu₃P)₂ (29 mg, 0.06 mmol) was added, the vessel evacuated and backfilled with nitrogen (3× cycles) and the mixture stirred at 50° C. for 17 hours. The resulting mixture was cooled to room temperature and partitioned between water (25 mL) and DCM (25 mL). The layers were separated and the aqueous layer was further extracted with DCM (25 mL). The combined organic fractions were dried over Na₂SO₄, filtered and concentrated in vacuo. Purification by chromatography on silica eluting with a gradient of 0 to 3% MeOH in DCM afforded a pale orange glass. This material was suspended in Et₂O (5 mL) and the resulting solid collected by filtration, washed with additional Et₂O (3×1 mL) then dried to afford the title compound as a yellow solid.

LC-MS (Method 8A): Rt 2.27 mins; MS m/z 461.5=[M+H]+

¹H NMR (400 MHz, DMSO) δ 8.40 (d, J=9.4 Hz, 1H), 8.09 (br t, J=1.5 Hz, 1H), 7.93 (br dt, J=7.9, 1.5 Hz, 1H), 7.91-7.85 (m, 2H), 7.83 (d, J=9.4 Hz, 1H), 7.65 (apr t, J=7.8 Hz, 1H), 7.35 (s, 2H), 4.66 (s, 1H), 3.87 (dq, J=9.1, 6.7 Hz, 1H), 1.16 (s, 3H), 1.15-1.11 (m, 6H).

### Example 5

### 2-(3-Cyanophenyl)-N-[(1S)-2-hydroxy-1,2-dimethyl-propyl]-3-(4-methylquinazolin-6-yl)imidazo[1,2-b]pyridazine-6-carboxamide

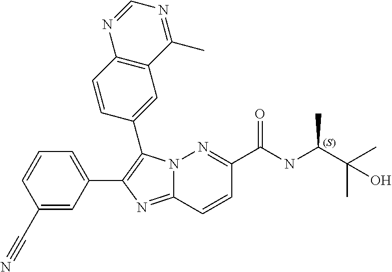

A solution of K₂CO₃ (310 mg, 2.24 mmol) in water (1 mL) was added to a solution of 3-bromo-2-(3-cyanophenyl)-N-[(1S)-2-hydroxy-1,2-dimethyl-propyl]imidazo[1,2-b]pyridazine-6-carboxamide (Intermediate D) (240 mg, 0.56 mmol) and 4-methyl-6-(4,4,5,5-tetramethyl-1,3,2-dioxaborolan-2-yl)quinazoline (Intermediate G)(303 mg, 1.12 mmol) in 1,4-dioxane (4 mL) and the resulting mixture de-oxygenated via nitrogen sparging for 10 mins. Pd(tBu₃P)₂ (29 mg, 0.06 mmol) was added, the vessel evacuated and backfilled with nitrogen (3× cycles) and the mixture was stirred at 50° C. for 1.75 hours. The mixture was cooled to room temperature before being diluted with EtOAc (40 mL), THF (40 mL) and MeOH (5 mL) and washed with water (40 mL). The organic layer was separated and the aqueous layer further extracted with a mixture of EtOAc (40 mL) and MeOH (5 mL). The combined organic fractions were dried over Na₂SO₄ and concentrated in vacuo. Purification by chromatography on silica eluting with a gradient of 0 to 5% MeOH in DCM afforded a yellow-green solid. The crude product was suspended in warm MeCN (5 mL) and the solids collected by filtration before being washed with additional MeCN (3×1 mL). The crude product was suspended in 1:1 DCM/EtOAc (2 mL) and the solids collected by filtration, washed with additional DCM (2×1 mL) and dried to afford a yellow solid. Finally, the crude product was suspended in a boiling mixture of MeCN (10 mL), toluene (2 mL) and MeOH (1 mL) for 30 mins before being left to cool to room temperature and stand overnight. The resulting mixture was filtered and the collected solids washed with acetone (2 mL) and dried to afford the title compound as a yellow solid.

LC-MS-1 (Method 8B): Rt 3.07 mins; MS m/z 492.5=[M+H]+

¹H NMR (400 MHz, DMSO) δ 9.20 (s, 1H), 8.78 (s, 1H), 8.44 (d, J=9.2 Hz, 1H), 8.11 (br t, J=1.5 Hz, 1H), 8.08 (br s, 2H), 7.92 (br dt, J=8.0, 1.5 Hz, 1H), 7.89-7.85 (m, 2H), 7.83 (d, J=9.2 Hz, 1H), 7.60 (apr t, J=7.9 Hz, 1H), 4.57 (s, 1H), 3.86 (dq, J=8.9, 6.7 Hz, 1H), 2.91 (s, 3H), 1.10-1.05 (m, 6H), 1.03 (s, 3H).

### Example 6

### 2-(3-Cyanophenyl)-N-[(1S)-2-hydroxy-1,2-dimethyl-propyl]-3-[2-(hydroxymethyl)-6-methyl-4-pyridyl]imidazo[1,2-b]pyridazine-6-carboxamide

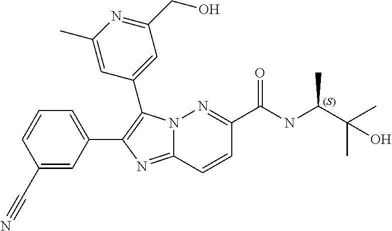

### Step 1: 3-[2-[[tert-Butyl(dimethyl)silyl]oxymethyl]-6-methyl-4-pyridyl]-2-(3-cyanophenyl)-N-[(1 S)-2-hydroxy-1,2-dimethyl-propyl]imidazo[1,2-b]pyridazine-6-carboxamide

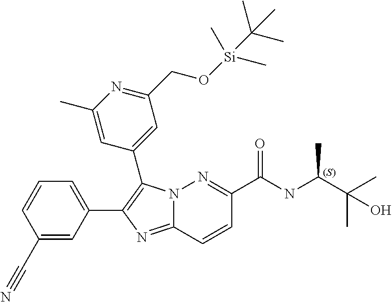

A solution of K₂CO₃ (129 mg, 0.93 mmol) in water was added to a mixture of 3-bromo-2-(3-cyanophenyl)-N-[(1S)-2-hydroxy-1,2-dimethyl-propyl]imidazo[1,2-b]pyridazine-6-carboxamide (Intermediate D)(100 mg, 0.23 mmol) and tert-butyl-dimethyl-[[6-methyl-4-(4,4,5,5-tetramethyl-1,3,2-dioxaborolan-2-yl)-2-pyridyl]methoxy]silane (Intermediate H)(261 mg, 0.47 mmol) in 1,4-dioxane (2 mL) and the resulting mixture de-oxygenated via nitrogen sparging for 10 mins. Pd(tBu₃P)₂ (12 mg, 0.02 mmol) was added and the vessel evacuated and backfilled with nitrogen (3× cycles) before the mixture was stirred at 50° C. for 1 h. The mixture was cooled to room temperature and partitioned between EtOAc (10 mL) and water (10 mL). The organic layer was separated and the aqueous layer further extracted with EtOAc (2×10 mL). The combined organic fractions were dried over Na₂SO₄ and concentrated in vacuo. Purification by chromatography on silica eluting with a gradient of 1 to 4% MeOH in DCM afforded the title compound as a yellow solid.

LC-MS (Method 3B): Rt 2.23 mins; MS m/z 585.4=[M+H]+

¹H NMR (500 MHz, DMSO) δ 8.40 (d, J=9.4 Hz, 1H), 8.01 (t, J=1.7 Hz, 1H), 7.93 (dt, J=7.9, 1.5 Hz, 1H), 7.91-7.86 (m, 2H), 7.84 (d, J=9.4 Hz, 1H), 7.83 (d, J=1.5 Hz, 1H), 7.64 (t, J=7.8 Hz, 1H), 7.17 (s, 1H), 4.68 (s, 2H), 4.67 (s, 1H), 3.88 (dt, J=9.0, 6.7 Hz, 1H), 2.58 (s, 3H), 1.21-1.11 (m, 9H), 0.71 (s, 9H), −0.04 (d, J=2.0 Hz, 6H).

### Step 2: 2-(3-Cyanophenyl)-N-[(1S)-2-hydroxy-1,2-dimethyl-propyl]-3-[2-(hydroxymethyl)-6-methyl-4-pyridyl]imidazo[1,2-b]pyridazine-6-carboxamide

1M TBAF in THF (0.44 mL, 0.4400 mmol) was added to a solution of 3-[2-[[tert-butyl(dimethyl)silyl]oxymethyl]-6-methyl-4-pyridyl]-2-(3-cyanophenyl)-N-[(1S)-2-hydroxy-1,2-dimethyl-propyl]imidazo[1,2-b]pyridazine-6-carboxamide (step 1)(164 mg, 0.22 mmol) in THF (2 mL) and the mixture stirred at room temperature for 20 mins. Purification by chromatography on silica eluting with a gradient of 1 to 5% MeOH in DCM afforded the title compound as a yellow solid.

LC-MS (Method 8B): Rt 3.69 mins; MS m/z 471.4=[M+H]+

¹H NMR (500 MHz, DMSO) δ 8.41 (d, J=9.4 Hz, 1H), 8.10 (t, J=1.7 Hz, 1H), 7.95 (dt, J=8.0, 1.4 Hz, 1H), 7.90 (dt, J=7.7, 1.4 Hz, 1H), 7.87-7.81 (m, 2H), 7.65 (t, J=7.8 Hz, 1H), 7.55 (d, J=1.5 Hz, 1H), 7.45 (d, J=1.5 Hz, 1H), 5.31 (t, J=5.8 Hz, 1H), 4.73 (s, 1H), 4.56 (d, J=6.1 Hz, 2H), 3.89 (dq, J=9.1, 6.7 Hz, 1H), 2.53 (s, 3H), 1.16 (s, 3H), 1.15-1.11 (m, 6H).

### Preparation of Intermediates

### Intermediate A

### Methyl 3-bromo-2-(3-cyanophenyl)imidazo[1,2-b]pyridazine-6-carboxylate

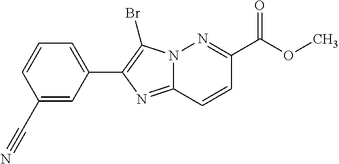 [SMILES: COC(=O)c1ccc2nc(-c3cccc(C#N)c3)c(Br)n2n1]

### Step 1: Methyl 2-(3-cyanophenyl)imidazo[1,2-b]pyridazine-6-carboxylate

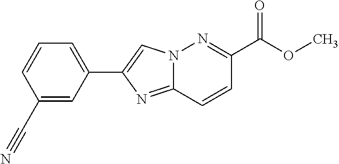 [SMILES: COC(=O)c1ccc2nc(-c3cccc(C#N)c3)cn2n1]

IMS (280 mL) was added to methyl 6-aminopyridazine-3-carboxylate (6.3 g, 41.14 mmol) and the mixture briefly stirred to yield a fine semi-suspension. NaHCO₃ (5.18 g, 61.71 mmol) was added followed by 3-(2-bromoacetyl)benzonitrile (11.06 g, 49.37 mmol) and the reaction mixture was heated at reflux (80° C.) for 5 h. After cooling to room temperature, the resulting suspension was filtered and the solids washed with IMS (2×150 mL), water (3×150 mL), IMS (2×150 mL) and diethyl ether (2×150 mL). The solid was dried in vacuo to afford the title compound as a cream solid.

LC-MS (Method 5B): Rt 2.73 mins; MS m/z 279.1=[M+H]+

¹H NMR (500 MHz, DMSO) δ 9.16 (s, 1H), 8.50 (br t, J=1.5 Hz, 1H), 8.41 (br dt, J=7.9, 1.5 Hz, 1H), 8.31 (d, J=9.5 Hz, 1H), 7.87 (br dt, J=7.8, 1.5 Hz, 1H), 7.77 (d, J=9.5 Hz, 1H), 7.72 (apr t, J=7.8 Hz, 1H), 3.97 (s, 3H).

### Step 2: Methyl 3-bromo-2-(3-cyanophenyl)imidazo[1,2-b]pyridazine-6-carboxylate

N-Bromosuccinimide (8.7 g, 48.89 mmol) was added portion-wise over 3 minutes to a stirred suspension of methyl 2-(3-cyanophenyl)imidazo[1,2-b]pyridazine-6-carboxylate (step 1)(12.37 g, 44.45 mmol) in DMF (178 mL) and the resulting mixture stirred at room temperature for 30 mins. Further portions of DMF (40 mL+80 mL) were added to aid mixing and stirring continued for a total of 2 h 30 mins. The mixture was poured into vigorously stirring water (1200 mL) and the resulting suspension stirred for 30 mins, then filtered. The solid was washed with water (3×300 mL), IMS (300 mL), diethyl ether (2×300 mL) and dried in vacuo to afford the title compound as a pale yellow solid.

LC-MS (Method 5B): Rt 3.07 mins; MS m/z 356.9/358.9=[M+H]+

¹H NMR (500 MHz, DMSO) δ 8.49 (s, 1H), 8.47 (d, J=8.3 Hz, 1H), 8.37 (d, J=9.5 Hz, 1H), 7.96 (d, J=7.8 Hz, 1H), 7.86 (d, J=9.5 Hz, 1H), 7.79 (apr t, J=7.9 Hz, 1H), 3.99 (s, 3H)

### Intermediate B

### 3-Bromo-2-(3-cyanophenyl)imidazo[1,2-b]pyridazine-6-carboxylic acid

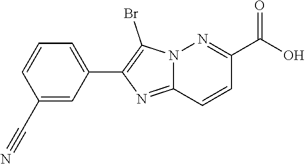 [SMILES: N#Cc1cccc(-c2nc3ccc(C(=O)O)nn3c2Br)c1]

To a solution of ethyl 3-bromo-2-(3-cyanophenyl)imidazo[1,2-b]pyridazine-6-carboxylate (Intermediate A)(248 mg, 0.67 mmol) in THF (6 mL) was added a solution of LiOH (48 mg, 2.01 mmol) in water (2 mL) and the mixture was stirred at room temperature for 45 mins. The resulting mixture was partitioned between water (20 mL) and EtOAc (20 mL). The organic layer was separated and the aqueous portion was acidified to pH 1 with 2M HCl. The mixture was concentrated in vacuo to afford the title compound as a yellow solid.

LC-MS (Method 5A): Rt 1.12 mins; MS m/z 343.0/345.0=[MH]+

¹H NMR (500 MHz, DMSO-d₆) δ 8.51 (s, 1H), 8.49 (d, J=8.5 Hz, 1H), 8.35 (d, J=9.5 Hz, 1H), 7.97 (d, J=7.5 Hz, 1H), 7.86 (d, J=9.5 Hz, 1H), 7.80 (t, J=7.9 Hz, 1H). Exchangeable proton not observed.

### Intermediate C

### 3-Bromo-2-(3-cyanophenyl)-N-(2-hydroxy-2-methyl-propyl)imidazo[1,2-b]pyridazine-6-carboxamide

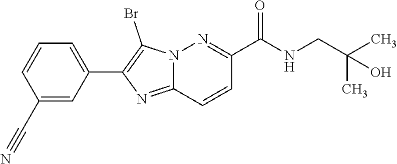 [SMILES: CC(C)(O)CNC(=O)c1ccc2nc(-c3cccc(C#N)c3)c(Br)n2n1]

The preparation of the title compound is described in Example 1.

### Intermediate D

### 3-Bromo-2-(3-cyanophenyl)-N-[(1S)-2-hydroxy-1,2-dimethyl-propyl]imidazo[1,2-b]pyridazine-6-carboxamide

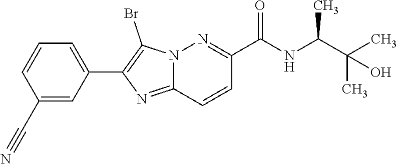 [SMILES: C[C@H](NC(=O)c1ccc2nc(-c3cccc(C#N)c3)c(Br)n2n1)C(C)(C)O]

To a mixture of 3-bromo-2-(3-cyanophenyl)imidazo[1,2-b]pyridazine-6-carboxylic acid (Intermediate B) (400 mg, 1.17 mmol) and (3S)-3-amino-2-methyl-butan-2-ol hydrochloride (163 μL, 1.75 mmol) in DMF (5 mL) was added DIPEA (1015 μL, 5.83 mmol) followed by dropwise addition of T3P® (50% in DMF) (1.64 mL, 2.33 mmol). The reaction mixture was stirred at room temperature for 2 h and then poured into water (40 mL). The resulting suspension was collected by filtration, dissolved in acetone and dried in vacuo to afford the title compound as a yellow solid.

LC-MS (Method 3.5B): Rt 1.87 mins; MS m/z 427.9/430.3=[M+H]+

¹H NMR (500 MHz, DMSO-d6) δ 8.51 (s, 1H), 8.49 (d, J=7.8 Hz, 1H), 8.37 (d, J=9.4 Hz, 1H), 8.06 (d, J=9.1 Hz, 1H), 7.96 (d, J=7.8 Hz, 1H), 7.85 (d, J=9.4 Hz, 1H), 7.80 (t, J=7.8 Hz, 1H), 4.77 (s, 1H), 3.96 (dq, J=9.1, 6.6 Hz, 1H), 1.22-1.12 (m, 9H).

### Intermediate E

### 3-Bromo-2-(3-cyanophenyl)-N-[(1R)-2-hydroxy-1,2-dimethyl-propyl]imidazo[1,2-b]pyridazine-6-carboxamide

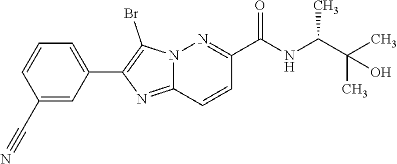 [SMILES: C[C@@H](NC(=O)c1ccc2nc(-c3cccc(C#N)c3)c(Br)n2n1)C(C)(C)O]

T3P® (50% in DMF) (1.64 mL, 2.33 mmol) was added dropwise to a stirred suspension of 3-bromo-2-(3-cyanophenyl)imidazo[1,2-b]pyridazine-6-carboxylic acid (Intermediate B) (400 mg, 1.17 mmol), (3R)-3-amino-2-methyl-butan-2-ol (163 μL, 1.75 mmol) and DIPEA (1.42 mL, 8.16 mmol) in anhydrous DMF (5 mL) under nitrogen atmosphere and the mixture stirred at room temperature for 1 h 45 mins. The resulting mixture was added to stirring water (40 mL) and the suspension stirred for 1 h. The solids were collected by filtration, washed with water (3×15 mL) and dried in vacuo to afford the title compound as a cream solid.

LC-MS (Method 3A): Rt 1.76 mins; MS m/z 428.2/430.2=[M+H]+

¹H NMR (500 MHz, DMSO) δ 8.52-8.48 (m, 2H), 8.37 (d, J=9.5 Hz, 1H), 8.06 (d, J=9.2 Hz, 1H), 7.96 (d, J=7.7 Hz, 1H), 7.85 (d, J=9.5 Hz, 1H), 7.80 (apr t, J=7.8 Hz, 1H), 4.77 (s, 1H), 3.99-3.93 (m, 1H), 1.22-1.17 (m, 6H), 1.15 (s, 3H).

### Intermediate F

### 4-(4,4,5,5-Tetramethyl-1,3,2-dioxaborolan-2-yl)-2,6-bis(trideuteriomethyl)pyridine

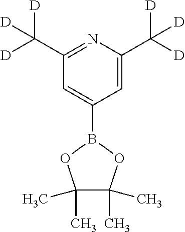 [SMILES: [2H]C([2H])([2H])c1cc(B2OC(C)(C)C(C)(C)O2)cc(C([2H])([2H])[2H])n1]

### Step 1: 2,6-Bis(trideuteriomethyl)pyridine

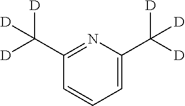 [SMILES: [2H]C([2H])([2H])c1cccc(C([2H])([2H])[2H])n1]

2,6-Dichloropyridine (5.0 g, 33.79 mmol) and NiCl₂(dppp) (916 mg, 1.69 mmol) were placed under nitrogen and anhydrous THF (68 mL) was added via syringe. The mixture was cooled to 0° C. and treated dropwise with a solution of methyl-d3-magnesium iodide (1M in Et₂O) (100 mL, 100 mmol) over 20 mins. The reaction mixture was heated at reflux for 1.5 h and allowed to cool to room temperature. The reaction was quenched by cautious addition of MeOH (15 mL) followed by 1M aqueous HCl (125 mL). The resulting bi-phasic mixture was stirred vigorously for 5 mins until all of the solids had dissolved and effervescence had fully ceased. The mixture was diluted with Et₂O (125 mL) and the layers separated. The organic layer was extracted with water (125 mL) and the combined aqueous layers adjusted to pH 10 with 10% aqueous NaOH. The resulting mixture was extracted with EtOAc (2×300 mL) and the combined organic extracts were washed with brine (200 mL), dried over Na₂SO₄, and cautiously concentrated at 35-38° C. and 170-190 mbar then briefly at 40° C. and 100 mbar to afford the crude material as a pale orange-brown oil. The oil was purified by vacuum distillation (120-140° C. under low vacuum) to afford the title compound as a clear, colourless oil.

LC-MS (Method 2B): Rt 1.05 mins; MS m/z 114.0=[M+H]+

1H NMR (500 MHz, DMSO-d6) δ 7.54 (t, J=7.6 Hz, 1H), 7.02 (d, J=7.6 Hz, 2H)

### Step 2: 4-(4,4,5,5-Tetramethyl-1,3,2-dioxaborolan-2-yl)-2,6-bis(trideuteriomethyl)pyridine

Methoxy(cyclooctadiene)iridium(I) dimer (789 mg, 1.19 mmol), 4,4′-di-tert-butyl-2,2′-dipyridyl (639 mg, 2.38 mmol) and bis(pinacolato)diboron (10.07 g, 39.67 mmol) were placed in a sealed flask and the contents evacuated and backfilled with nitrogen (3× cycles). Freshly de-oxygenated anhydrous cyclohexane (50 mL) was added and the mixture was stirred vigorously for 1 h at room temperature before a solution of 2,6-bis(trideuteriomethyl)pyridine (step 1) (4.49 g, 39.67 mmol) in de-oxygenated anhydrous cyclohexane (50 mL) was added via syringe under nitrogen. The resulting mixture was stirred at room temperature for 19.5 h. Additional bis(pinacolato)diboron (2.01 g, 7.93 mmol) in de-oxygenated anhydrous cyclohexane (10 mL) was added via syringe under nitrogen and the mixture was stirred at room temperature for 2 h. Additional methoxy(cyclooctadiene)iridium(I) dimer (263 mg, 0.40 mmol) and 4,4′-di-tert-butyl-2,2′-dipyridyl (213 mg, 0.79 mmol) were added and stirring continued under nitrogen at room temperature for a further 17.5 h after which time the mixture was concentrated in vacuo to afford the title compound as a dark brown solid.

LC-MS (Method 2B): Rt 0.92 mins; MS m/z 240.1=[M+H]+

¹H NMR (500 MHz, DMSO-d6) δ 7.22 (s, 2H), 1.29 (s, 12H).

### Intermediate G

### 4-Methyl-6-(4,4,5,5-tetramethyl-1,3,2-dioxaborolan-2-yl)quinazoline

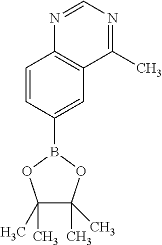

A solution of 6-bromo-4-methyl-quinazoline (500 mg, 2.24 mmol) and bis(pinacolato)diboron (683 mg, 2.69 mmol) in 1,4-dioxane (10 mL) was sparged with nitrogen for 10 mins before Pd(dppf)Cl₂ (164.01 mg, 0.2200 mmol) and KOAc (549.94 mg, 5.6 mmol) were added. The vessel was evacuated and backfilled with nitrogen (3× cycles) and the mixture stirred at 80° C. for 18 hours. The mixture was cooled to room temperature, diluted with EtOAc (10 mL) and filtered through Celite® (filter material) with the combined filtrate concentrated in vacuo to afford the crude title compound as a dark brown residue.

LC-MS (Method 2B): Rt 0.68 mins; MS m/z 271.3=[M+H]+

¹H NMR (500 MHz, DMSO) δ 9.14 (s, 1H), 8.52 (s, 1H), 8.16 (dd, J=8.4, 1.4 Hz, 1H), 7.96 (d, J=8.4 Hz, 1H), 2.96 (s, 3H), 1.36 (s, 12H).

### Intermediate H

### tert-Butyl-dimethyl-[[6-methyl-4-(4,4,5,5-tetramethyl-1,3,2-dioxaborolan-2-yl)-2-pyridyl]methoxy]silane

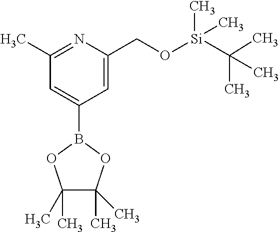 [SMILES: Cc1cc(B2OC(C)(C)C(C)(C)O2)cc(CO[Si](C)(C)C(C)(C)C)n1]

### Step 1: (4-Bromo-6-methyl-2-pyridyl)methoxy-tert-butyl-dimethyl-silane

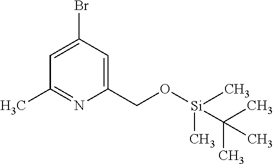 [SMILES: Cc1cc(Br)cc(CO[Si](C)(C)C(C)(C)C)n1]

To a solution of (4-bromo-6-methyl-2-pyridyl)methanol (2.0 g, 9.9 mmol) in DMF (15 mL) at 0° C. was added imidazole (0.03 mL, 12.87 mmol) followed by tert-butyldimethylsilyl chloride (1.11 mL, 10.89 mmol) and the reaction mixture was stirred at room temperature for 1 h. The resulting mixture was partitioned between EtOAc (150 mL) and water (150 mL) and the layers separated. The aqueous layer was further extracted with EtOAc (100 mL) and the combined organic extracts were washed with sat. NaHCO₃ solution (150 mL), brine (150 mL), dried over MgSO₄ and concentrated in vacuo to afford the title compound as a yellow oil which slowly crystallised to give a yellow solid.

LC-MS (Method 3B): Rt 2.52 mins; MS m/z 316.1/318.1=[M+H]+

¹H NMR (500 MHz, DMSO-d6) δ 7.45 (d, J=0.8 Hz, 1H), 7.38 (s, 1H), 4.70 (s, 2H), 2.43 (s, 3H), 0.91 (s, 9H), 0.10 (s, 6H).

### Step 2: tert-Butyl-dimethyl-[[6-methyl-4-(4,4,5,5-tetramethyl-1,3,2-dioxaborolan-2-yl)-2-pyridyl]methoxy]silane

A solution of (4-bromo-6-methyl-2-pyridyl)methoxy-tert-butyl-dimethyl-silane (step 1) (2.96 g, 9.35 mmol), bis(pinacolato)diboron (3.56 g, 14.02 mmol), potassium acetate (5.16 g, 37.39 mmol) and Pd(dppf)Cl₂ (889 mg, 1.22 mmol) in 1,4-dioxane (45 mL) was heated to 80° C. overnight. The resulting mixture was concentrated in vacuo and the residue was dissolved in hexane (adding a small volume of DCM to aid dissolution). The mixture was filtered through Celite® (filter material) and concentrated in vacuo to afford the title compound as a brown solid.

¹H NMR (500 MHz, DMSO-d6) δ 7.46 (s, 1H), 7.31 (s, 1H), 4.71 (s, 2H), 2.44 (s, 3H), 1.30 (s, 12H), 0.91 (s, 9H), 0.09 (s, 6H).

### BIOLOGICAL EXAMPLES

### Biological Example 1—Adenosine Receptor Time-Resolved Fluorescence Resonance Energy Transfer (TRFRET) Binding Assay

All FRET binding experiments were conducted at room temperature in white 384-well plates, in assay binding buffer containing 1× LabMed (Cisbio, France), 100 μg/mL saponin, 1% DMSO and 0.02% pluronic acid. Binding of the fluorescently labelled Adenosine receptor antagonist XAC (CA200645, FRET acceptor) to terbium-labelled A1, A2a, A2b and A3 adenosine receptors (FRET donors) was detected by time-resolved FRET due to the close proximity of the donor and acceptor in a binding event. To investigate the ability of unlabelled test compounds to bind to Adenosine A1, A2a, A2b and A3 receptors, dose response curves were constructed that determined the ability of a range of concentrations to inhibit the binding of 30 nM CA200645 to the A2b receptor and 100 nM CA200645 to the A1, A2a, and A3 receptor.

Serial dilution (1:3 dilutions) of test compounds in neat DMSO and transfer of a 400 nL sample of test compound into the assay plate was carried out using the Mosquito (TTP Labtech, UK). The compound samples were incubated for 2 hours at room temperature with a fixed concentration of CA200645 defined for each receptor (see above) and CHO cell membranes containing the human Adenosine A1 (0.5 μg/well), A2a (0.3 μg/well), A2b (1 μg/well) or A3 (1 μg/well) receptor in 40 μL of assay buffer. Total and non-specific binding of CA200645 was determined in the absence and presence of 10 μM XAC, respectively. Following 2 hours incubation, the level of CA200645 binding was detected on a Pherastar FSX (BMG Labtech, Germany) using standard TR-FRET settings. The terbium donor was excited with three laser flashes at a wavelength of 337 nm, and donor and acceptor emission was detected at 620 nm and 665 nm wavelengths, respectively. FRET ratios were obtained by multiplying the acceptor/donor ratio value by 10,000. Specific binding was determined by subtracting the non-specific binding FRET ratio from the total binding FRET ratio. Compound IC50 curves were analysed using GraphPad Prism 7.0 (GraphPad, USA) and Ki affinity values were determined from the obtained IC50 values using the method of Cheng and Prusoff. The results are presented in Table 1.

TABLE 1

| Example | A2a Ki | | A2b Ki | |
|---|---|---|---|---|
| Number | (nM) | A1 Ki (nM) | (nM) | A3 Ki (nM) |
| 1.0 | 10.9 | 4000 | 500 | >1000 |
| 2.0 | 3.9 | 1826 | 1579 | >1000 |
| 2.1 | 0.79 | 667 | 212 | >1000 |
| 2.2 | 0.50 | 363 | 27 | >1000 |
| 2.3 | 0.46 | 95 | 18 | >1000 |
| 2.4 | 2.3 | 286 | 156 | >1000 |
| 2.5 | 0.32 | 78 | 34 | >1000 |
| 2.6 | 0.17 | 30 | 18 | >1000 |
| 2.7 | 0.34 | 324 | 79 | >1000 |
| 2.8 | 0.19 | 243 | 285 | >1000 |
| 2.9 | 0.20 | 96 | 20 | >1000 |
| 3.0 | 0.16 | 71 | 31 | >1000 |
| 6 | 0.68 | 120 | 49 | >1000 |

### Biological Example—CD3/CD28 Stimulated IL-2 Release NECA Reversal Assay in Human PBMCs

Blood is drawn from healthy volunteers using sodium citrate as the anticoagulant (0.3% final concentration). After centrifugation of the blood over Histopaque-1077, PBMCs are collected from the Histopaque/plasma interface and washed twice in PBS (300 g for 10 mins at room temp). Cells are plated at 50,000 cells/well in 150 μl RPMI/10% FCS in 96-well cell culture plates that have been precoated with 1 ug/ml CD3 antibody. 50 μl diluted compound mix is added to the cells, to obtain final concentrations of 1 μg/ml CD28 antibody, 1 uM NECA and 0.003-10 μM adenosine receptor antagonist. Assay plates are incubated for 24 hours at 37° C. in a humidified incubator. Culture supernatant is tested for IL-2 levels using the human IL-2 Tissue Culture Kit (Meso Scale Discovery). Data for dose-response curves is calculated as % inhibition with 100% inhibition defined from no agonist control wells (+CD3/28-NECA)

TABLE 2

| Example | IL-2 IC50 (μM) |
|---|---|
| 2.1 | 0.0138 |
| 2.2 | 0.039 |
| 2.3 | 0.0073 |
| 2.4 | 0.0135 |
| 2.5 | 0.0011 |
| 2.6 | 0.0006 |
| 2.7 | 0.0105 |
| 2.8 | 0.0046 |
| 2.9 | 0.0053 |
| 3 | 0.0017 |

### Biological Example—Measurement of pCREB in CD8+ T Cells in Human Whole Blood

Heparinised human whole blood was pre-incubated at 37° C. with serial dilutions of A2a antagonists for 20 min. and the phosphodiesterase inhibitor rolipram to amplify the pCREB response. The adenosine receptor agonist NECA is then added at a final concentration of 3 μM and following a 60 min incubation the blood is fixed and red blood cells lysed. White blood cells are isolated, permeabilized and stained with directly conjugated fluorescent antibodies to phospho-CREB (Alexa Fluor 488) and CD8 (Alexa Fluor 647) and the level of phospho-CREB in CD8+ T cells is measured by FACS using a BD Accuri C6 Flow Cytometer.

TABLE 3

| | pCREB |
|---|---|
| Example | IC50 (μM) |
| 2.1 | 0.122 |
| 2.2 | 0.496 |
| 2.3 | 0.282 |
| 2.4 | 2.038 |
| 2.5 | 0.105 |
| 2.6 | 0.058 |
| 2.7 | 0.499 |
| 2.8 | 0.472 |
| 2.9 | 0.168 |
| 3 | 0.061 |

### REFERENCES

1. Sukari A Nagasaka M AI-Hadidi A and Lum L G (2016). Anticancer Res. 36(11):5593-5606.
2. Vijayan D, Young A, Teng M W L, and Smyth M J (2017), Nat Rev Cancer. 17(12):709-724.
3. Houthuys, E, Marillier R, Deregnaucourt, T, Brouwer, M, Pirson, R, Marchante, J, et al (2016). SITC 2017 Conference, Maryland.
4. Gao Z W, Dong K, Zhang H Z (2014). “The roles of CD73 in cancer”. Biomed Res Int: 2014:460654.
5. Loi S, Pommey S, Haibe-Kains B, Beavis P A, Darcy P K, Smyth M J, et al. (2013), “CD73 promotes anthracycline resistance and poor prognosis in triple negative breast cancer” Proc Natl Acad Sci USA.;110(27):11091-6.
6. Deaglio S, Dwyer K M, Gao W, Friedman D, Usheva A, Erat A et al (2007). J. Exp Med. 204, No. 6, Jun. 11, 2007 1257-1265

### NUMBERED PARAGRAPHS

The following numbered paragraphs serve to define particular aspects and embodiments of the invention.

1. A compound, or pharmaceutically acceptable salt thereof, having the structural formula Ia shown below:

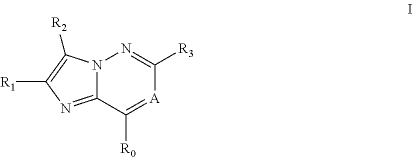

wherein:
R₀ is hydrogen or deuterium;
R₁ is selected from aryl or heteroaryl,
   wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, (CH₂)_{q}1N(R_{1C})C(O)R_{1B}, (CH₂)_{q}1S(O)ₚR_{1B} (where p is 0, 1 or 2), (CH₂)_{q1}SO₂N(R_{1C})R_{1B}, or (CH₂)_{q1}N(R_{1C})SO₂R_{1B},
   and wherein q1 is 0, 1, 2 or 3 and R_{1B} and R_{1C} are each independently selected from hydrogen, (1-4C)alkyl, (3-6C)cycloalkyl or (3-6C)cycloalkyl(1-2C)alkyl;
R₂ is selected from hydrogen, cyano, halo, (1-4C)alkyl, (1-4C)haloalkyl, C(O)OR_{2A}, C(O)NR_{2A}R_{2B}, aryl, heterocyclyl, heteroaryl, (2-6C)alkenyl, (2-6C)alkynyl or (1-4C)alkanoyl;
   wherein R_{2A} and R_{2B} are each independently selected from hydrogen, (1-4C)alkyl, (1-4C)alkoxy, (3-6C)cycloalkyl or (3-6C)cycloalkyl(1-2C)alkyl, or, in the CONR_{2A}R_{2B} group, R_{2A} and R_{2B} are linked such that, together with the nitrogen atom to which they are attached, they form a heterocyclic ring, and
   wherein any alkyl, alkenyl, alkynyl, alkanoyl, aryl, heteroaryl or heterocyclyl group is optionally substituted by one or more substituents independently selected from oxo, (1-4C)alkyl, halo, (1-4C)haloalkyl, (1-4C)haloalkoxy, amino, (1-4C)aminoalkyl, cyano, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{2D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D}, wherein q2 is 0, 1, 2 or 3; and wherein R_{2D} and R_{2E} are each independently selected from hydrogen, (1-4C)alkyl, (3-6C)cycloalkyl or (3-6C)cycloalkyl(1-2C)alkyl;
R₃ is selected from hydrogen, halo, cyano or a group of the formula:

-L-Y-L_{q}-Q
wherein:
   L is absent or (1-4C)alkylene optionally substituted by one or more substituents selected from (1-2C)alkyl or oxo;
   Y is absent or O, S, SO, SO₂, N(Rₐ), C(O), C(O)O, OC(O), C(O)N(Rₐ), N(Rₐ)C(O), C(O)N(Rₐ)—O—, N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein
   R_{c}, R_{d} and Rₑ are each independently selected from hydrogen, (1-6C)alkyl, (3-6C)cycloalkyl or (3-6C)cycloalkyl(1-2C)alkyl; or R_{c} and R_{d} are linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy; and/or
   Q is optionally substituted by one or more group(s) of the formula:

-L₁-L_{Q1}-W₁
wherein:
   L₁ is absent or (1-3C)alkylene optionally substituted by one or more substituents selected from (1-2C)alkyl or oxo;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
   W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
   and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups; or
   Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
A is selected from CR₄ and N,
   wherein R₄ is hydrogen, halo or (1-4C)alkyl optionally substituted by one or more substituents selected from halo, (1-4C)haloalkyl, (1-4C)haloalkoxy, (1-4C)aminoalkyl, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and R_{4A} and R_{4B} are each independently selected from hydrogen, (1-6C)alkyl, (3-6C)cycloalkyl or (3-6C)cycloalkyl(1-2C)alkyl;

and wherein any tertiary amine in a compound of formula I is optionally in the form of a N-oxide and any nitrogen atom in a heteroaryl ring is optionally in the form of an N-oxide;

and wherein any S atoms present in the heterocyclic ring may optionally be present as S(═O), S(═O)₂ or S(═O)(═NR_{z}) wherein R_{z} is selected from hydrogen, (1-3C)alkyl or (2-3C)alkanoyl.

2. A compound according to paragraph 1, or a pharmaceutically acceptable salt thereof, wherein R₁ is selected from aryl or heteroaryl, wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
q1 is 0, 1 or 2; and
R_{1B} and R_{1C} are each independently selected from hydrogen, (1-4C)alkyl, (3-6C)cycloalkyl or (3-6C)cycloalkyl(1-2C)alkyl.

3. A compound according to any one of the preceding paragraphs, or a pharmaceutically acceptable salt thereof, wherein R₁ is selected from aryl or heteroaryl, wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B}(where p is 0, 1 or 2), SO₂N(R_{1C})R_{1B}, or N(R_{1C})SO₂R_{1B} and wherein:
q1 is 0, 1 or 2; and
R_{1B} and R_{1C} are each independently selected from hydrogen, (1-2C)alkyl or (3-4C)cycloalkyl.

4. A compound according to any one of the preceding paragraphs, or a pharmaceutically acceptable salt thereof, wherein R₁ is selected from phenyl, furyl, pyridyl or oxazolyl, wherein a phenyl, furyl, pyridyl or oxazolyl ring is optionally substituted by one or more of halo, (1-2C)alkyl, (1-2C)alkoxy or cyano.

5. A compound according to any one of the preceding paragraphs, or a pharmaceutically acceptable salt thereof, wherein R₁ is selected from phenyl, furyl, pyridyl, or oxazolyl, wherein a phenyl, furyl, pyridyl or oxazolyl ring is optionally substituted by halo or cyano.

6. A compound according to any one of the preceding paragraphs, or a pharmaceutically acceptable salt thereof, wherein R₁ is 3-cyanophenyl.

7. A compound according to any one of the preceding paragraphs, or a pharmaceutically acceptable salt thereof, wherein R₂ is selected from hydrogen, cyano, halo, (1-4C)alkyl, (1-4C)haloalkyl, C(O)OR_{2A}, C(O)NR_{2A}R_{2B}, aryl, heteroaryl, heterocyclyl, (2-6C)alkenyl, (2-6C)alkynyl or (1-4C)alkanoyl;
wherein R_{2A} and R_{2B} are each independently selected from hydrogen, (1-4C)alkyl, (1-4C)alkoxy, (3-6C)cycloalkyl or (3-6C)cycloalkyl(1-2C)alkyl,
or, in the CONR_{2A}R_{2B} group, R_{2A} and R_{2B} are linked such that, together with the nitrogen atom to which they are attached, they form a 4-7 membered heterocyclic ring, and
wherein any alkyl, alkenyl, alkynyl, alkanoyl, aryl, heteroaryl or heterocyclyl group is optionally substituted by one or more substituents independently selected from (1-4C)alkyl, halo, (1-2C)haloalkyl, (1-2C)haloalkoxy, cyano, oxo, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{12D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D},
wherein q2 is 0, 1, or 2; and
wherein R_{2D} and R_{2E} are each independently selected from hydrogen, (1-2C)alkyl, (3-4C)cycloalkyl or (3-4C)cycloalkyl(1-2C)alkyl.

8. A compound according to any one of the preceding paragraphs, or a pharmaceutically acceptable salt thereof, wherein R₂ is selected from cyano, halo, methyl, CF₃, C(O)OR_{2A}, C(O)NR_{2A}R_{2B}, a 5 or 6-membered heteroaryl or (2-4C)alkanoyl,
wherein R_{2A} and R_{2B} are each independently selected from hydrogen or (1-4C)alkyl,
wherein any heteroaryl group is optionally substituted by one or more substituents independently selected from (1-2C)alkyl, halo, (1-2C)haloalkyl, (1-2C)haloalkoxy, cyano, (CH₂)_{q2}NR_{2D}R_{2E}, OR_{2D}, C(O)R_{2D}, C(O)OR_{2D}, OC(O)R_{2D}, C(O)N(R_{2E})R_{2D}, N(R_{2E})C(O)R_{12D}, S(O)ₚR_{2D} (where p is 0, 1 or 2), SO₂N(R_{2E})R_{2D}, or N(R_{2E})SO₂R_{2D}, wherein q2 is 0 or 1; and wherein R_{2D} and R_{2E} are each independently selected from hydrogen or (1-2C)alkyl.

9. A compound according to any one of the preceding paragraphs, or a pharmaceutically acceptable salt thereof, wherein R₂ is selected from halo or a 5 or 6-membered heteroaryl which is optionally substituted as defined above in any one of paragraphs 7 or 8.

10. A compound according to any one of the preceding paragraphs, or a pharmaceutically acceptable salt thereof, wherein R₂ is either:

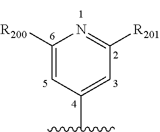

A)
wherein:
(i) R₂₀₀ and R₂₀₁ are each independently selected from (1-2C)alkyl, halo, (1-2C)haloalkyl, (1-2C)alkoxy, (1-2C)haloalkoxy, (1-2C)alkanoyl or cyano;
(ii) R₂₀₀ and R₂₀₁ are each independently selected from methyl, halo, di-fluoromethyl, trifluoromethyl, methoxy, acetyl or cyano; or
(iii) R₂₀₀ is methyl or chloro and R₂₀₁ is selected from methyl, halo, di-fluoromethyl, trifluoromethyl, methoxy, acetyl or cyano;

or

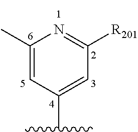

B)
wherein:
(i) R₂₀₁ is (1-2C)alkyl, halo, (1-2C)haloalkyl, (1-2C)alkoxy, (1-2C)haloalkoxy, (1-2C)alkanoyl or cyano;
(ii) R₂₀₁ is methyl, halo, di-fluoromethyl, trifluoromethyl, methoxy, acetyl or cyano; or
(iii) R₂₀₁ is methyl or chloro.

11. A compound according to any one of the preceding paragraphs, or a pharmaceutically acceptable salt thereof, wherein R₂ is:
bromo;
2-acetyl-6-methylpyridin-4-yl;
2,6-dimethylpyridin-4-yl;
2-Chloro-6-methylpyridin-4-yl
2-methyl-6-(trifluoromethyl)pyridine-4-yl;
2-methoxy-6-methyl-4-pyridyl;
2-(difluoromethyl)-6-methyl-4-pyridyl;
2-chloro-6-methyl-4-pyridyl;
2-chloro-6-methylpyridin-4-yl or 2,6-dimethylpyridin-4-yl.

12. A compound according to any one of the preceding paragraphs, or a pharmaceutically acceptable salt thereof, wherein R₃ is selected from hydrogen, halo, cyano or a group of the formula:

-L-Y-L_{q}-Q
wherein:
   L is absent or (1-4C)alkylene;
   Y is absent or O, S, SO, SO₂, N(Rₐ), C(O), C(O)O, OC(O), C(O)N(Rₐ), N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen, (1-6C)alkyl, (3-6C)cycloalkyl or (3-6C)cycloalkyl(1-2C)alkyl; and/or
   Q is optionally substituted by one or more group(s) of the formula:

-L₁-L_{Q1}-W₁
wherein:
   L₁ is absent or (1-3C)alkylene;
   L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and

W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;

and wherein any tertiary amine in a R₃ group is optionally in the form of a N-oxide.

13. A compound according to any one of the preceding paragraphs, or a pharmaceutically acceptable salt thereof, wherein R₃ is selected from hydrogen, halo, cyano or a group of the formula:

-L-Y-L_{q}-Q
wherein:
   L is absent or (1-2C)alkylene;
   Y is absent or O, N(Rₐ), C(O), C(O)O, C(O)N(Rₐ), N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
   L_{q} is absent or (1-4C)alkylene optionally substituted by one or more substituents selected from (1-2C)alkoxy, halo, cyano, amino or oxo; and
   Q is hydrogen, (1-6C)alkyl, aryl, (3-8)cycloalkyl, heteroaryl or heterocyclyl;
   wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R. and R_{d} are each independently selected from hydrogen or (1-6C)alkyl; and/or
   Q is optionally substituted by one or more group(s) of the formula:

-L₁-L_{Q1}-W₁
wherein:
   L₁ is absent or (1-2C)alkylene;
   L_{Q1} is absent; and
W₁ is hydrogen, (1-6C)alkyl, aryl, aryl(1-2C)alkyl, (3-8C)cycloalkyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), wherein Rₕ and Rᵢ are each independently selected from hydrogen or (1-4C)alkyl;
and wherein any tertiary amine in a R₃ group is optionally in the form of a N-oxide.

14. A compound according to any one of the preceding paragraphs, or a pharmaceutically acceptable salt thereof, wherein R₃ is a group of the formula:

-L-Y-L_{q}-Q
wherein:
   L is absent;
   Y is N(Rₐ) or C(O)N(Rₐ);
   L_{q} is absent; and
   Q is (1-6C)alkyl or (3-8C)cycloalkyl;
wherein Q is optionally further substituted by one or more substituent groups independently selected from halo, cyano, NR_{c}R_{d}, OR_{c}, C(O)Rₑ, C(O)ORₑ, C(O)N(R_{d})R_{c}, N(R_{d})C(O)R_{c}, S(O)ₚR_{c}(where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen or (1-6C)alkyl.

15. A compound according to any one of the preceding paragraphs, or a pharmaceutically acceptable salt thereof, wherein A is selected from CR₄ and N,
wherein R₄ is hydrogen, halo or (1-2C)alkyl optionally substituted by one or more substituents selected from halo, (1-2C)haloalkyl, (1-2C)haloalkoxy, amino, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and wherein R_{4A} and R_{4B} are each independently selected from hydrogen, (1-4C)alkyl, (3-4C)cycloalkyl or (3-4C)cycloalkyl(1-2C)alkyl.

16. A compound according to any one of the preceding paragraphs, or a pharmaceutically acceptable salt thereof, wherein A is selected from CR₄ and N, wherein R₄ is hydrogen, halo or (1-2C)alkyl optionally substituted by one or more substituents selected from halo.

17. A compound according to any one of the preceding paragraphs, or a pharmaceutically acceptable salt thereof, wherein A is selected from CR₄ and N, wherein R₄ is hydrogen, methyl or halo.

18. A compound of the formula:

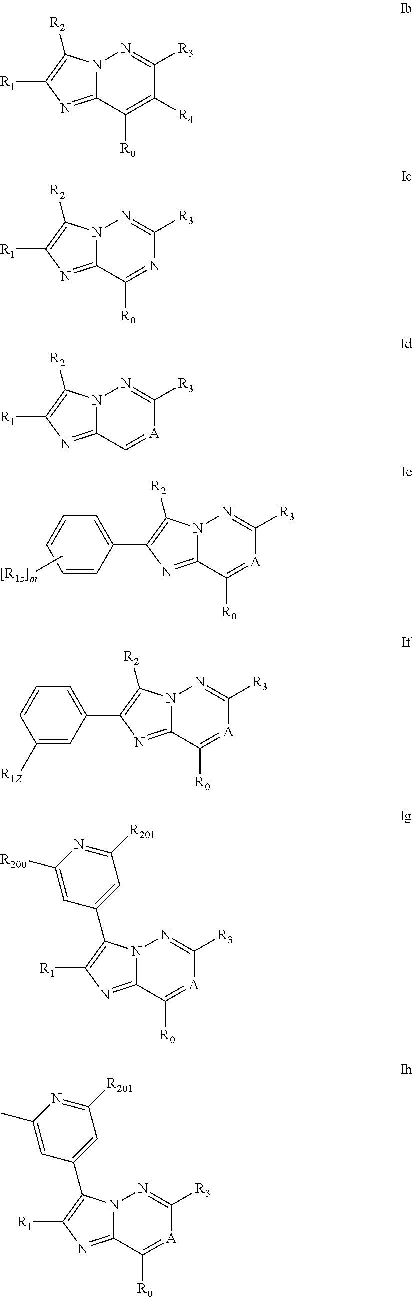

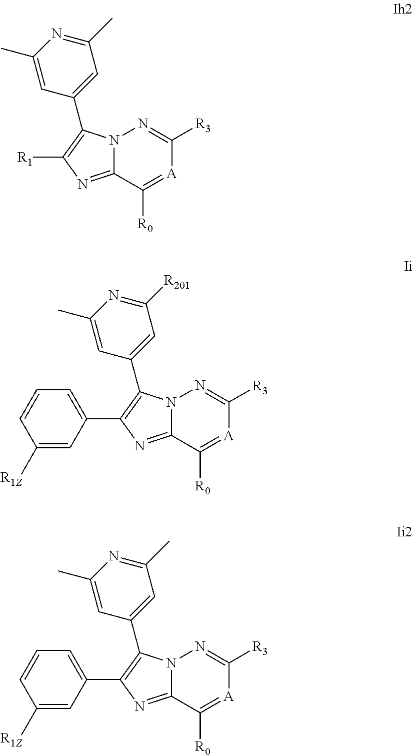

wherein A, R₀, R₁, R₂, R₃ and R_{1Z} are each as defined in any one of paragraphs 1 to 15;

m is 0, 1 or 2;

R₂₀₀ and R₂₀₁ are each as defined in paragraph 10;

or a pharmaceutically acceptable salt thereof.

19. A compound, or a pharmaceutically acceptable salt thereof, selected from any one of the following:
3-Bromo-2-(3-cyanophenyl)-N-(2-hydroxy-2-methyl-propyl)imidazo[1,2-b]pyridazine-6-carboxamide;
3-(2-Acetyl-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-3-(2,6-dimethyl-4-pyridyl)-N-(2-hydroxy-2-methyl-propyl)imidazo[1,2-b]pyridazine-6-carboxamide;
3-(2-Chloro-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-N-(2-hydroxy-2-methyl-propyl)-3-[2-methyl-6-(trifluoromethyl)-4-pyridyl]imidazo[1,2-b]pyridazine-6-carboxamide;
2-(3-Cyanophenyl)-N-(2-hydroxy-2-methyl-propyl)-3-(2-methoxy-6-methyl-4-pyridyl)imidazo[1,2-b]pyridazine-6-carboxamide;
2-(3-Cyanophenyl)-3-[2-(difluoromethyl)-6-methyl-4-pyridyl]-N-[(1S)-2-hydroxy-1,2-dimethyl-propyl]imidazo[1,2-b]pyridazine-6-carboxamide;
2-(3-Cyanophenyl)-N-[(1S)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1R)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1S)-2-hydroxy-1,2-dimethyl-propyl]-3-(2-methoxy-6-methyl-4-pyridyl)imidazo[1,2-b]pyridazine-6-carboxamide;
3-(2-Chloro-6-methyl-4-pyridyl)-2-(3-cyanophenyl)-N-[(1S)-2-hydroxy-1,2-dimethyl-propyl]imidazo[1,2-b]pyridazine-6-carboxamide;
2-(3-Cyanophenyl)-3-(2,6-dimethyl-4-pyridyl)-N-[(1S)-2-hydroxy-1,2-dimethyl-propyl]imidazo[1,2-b]pyridazine-6-carboxamide.

20. A pharmaceutical composition comprising a compound according to any one of paragraphs 1 to 19, or a pharmaceutically acceptable salt thereof, in admixture with a pharmaceutically acceptable diluent or carrier.

21. A compound according to any one of paragraphs 1 to 19, or a pharmaceutically acceptable salt thereof, or a pharmaceutical composition according to paragraph 20, for use in therapy.

22. A compound according to any one of paragraphs 1 to 19, or a pharmaceutically acceptable salt thereof, or a pharmaceutical composition according to paragraph 20, for use:
(i) in the treatment of a proliferative condition;
(ii) in the treatment of cancer;
(iii) in the treatment of cancer, wherein the compound or pharmaceutical composition is administered in combination with one or more additional anticancer agents;
(iv) in the treatment of cancer, wherein the compound or pharmaceutical composition is administered in combination with one or more additional anticancer agents selected from the group consisting of:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).

23. A method of treating a proliferative disorder in a patient in need of such treatment, the method comprising administering a therapeutically effective amount of a compound according to any one of paragraphs 1 to 19, or a pharmaceutically acceptable salt thereof, or a pharmaceutical composition according to paragraph 20.

24. A method of treating cancer in a patient in need of such treatment, the method comprising administering a therapeutically effective amount of a compound according to any one of paragraphs 1 to 19, or a pharmaceutically acceptable salt thereof, or a pharmaceutical composition according to paragraph 20.

25. A method of treating a proliferative disorder in a patient in need of such treatment, the method comprising administering a therapeutically effective amount of a compound according to any one of paragraphs 1 to 19, or a pharmaceutically acceptable salt thereof, or a pharmaceutical composition according to paragraph 20 in combination with one or more additional anticancer agents.

26. A method according to paragraph 25, wherein the one or more additional anticancer agents is selected from:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).

1. A compound, or pharmaceutically acceptable salt thereof, having the structural formula Ia shown below: 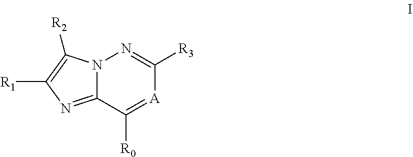
wherein:
R₀ is hydrogen or deuterium;
R₁ is selected from aryl or heteroaryl,
wherein R₁ is optionally substituted by one or more R_{1z} substituents independently selected from (1-4C)alkyl, halo, (1-4C)haloalkyl, (1-4C)haloalkoxy, cyano, (CH₂)_{q1}NR_{1B}R_{1C}, (CH₂)_{q1}OR_{1B}, (CH₂)_{q1}C(O)R_{1B}, (CH₂)_{q1}C(O)OR_{1B}, (CH₂)_{q1}OC(O)R_{1B}, (CH₂)_{q1}C(O)N(R_{1C})R_{1B}, (CH₂)_{q1}N(R_{1C})C(O)R_{1B}, (CH₂)_{q1}S(O)ₚR_{1B} (where p is 0, 1 or 2), (CH₂)_{q1}SO₂N(R_{1C})R_{1B}, or (CH₂)_{q1}N(R_{1C})SO₂R_{1B},
and wherein q1 is 0, 1, 2 or 3 and R_{1B} and R_{1C} are each independently selected from hydrogen, (1-4C)alkyl, (3-6C)cycloalkyl or (3-6C)cycloalkyl(1-2C)alkyl;
R₂ is selected from hydrogen, cyano, halo, (1-4C)alkyl, (1-4C)haloalkyl, C(O)OR_{2A}, C(O)NR_{2A}R_{2B}, aryl, heterocyclyl, heteroaryl, (2-6C)alkenyl, (2-6C)alkynyl or (1-4C)alkanoyl;
wherein R_{2A} and R_{2B} are each independently selected from hydrogen, (1-4C)alkyl, (1-4C)alkoxy, (3-6C)cycloalkyl or (3-6C)cycloalkyl(1-2C)alkyl, or, in the CONR_{2A}R_{2B} group, R_{2A} and R_{2B} are linked such that, together with the nitrogen atom to which they are attached, they form a heterocyclic ring, and
wherein any alkyl, alkenyl, alkynyl, alkanoyl, aryl, heteroaryl or heterocyclyl group is optionally substituted by one or more substituents independently selected from oxo, (1-4C)alkyl, halo, (1-4C)haloalkyl, (1-4C)haloalkoxy, amino, (1-4C)aminoalkyl, cyano, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{2D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D}, wherein q2 is 0, 1, 2 or 3; and wherein R_{2D} and R_{2E} are each independently selected from hydrogen, (1-4C)alkyl, (3-6C)cycloalkyl or (3-6C)cycloalkyl(1-2C)alkyl;
R₃ is selected from hydrogen, halo, cyano or a group of the formula:
-L-Y-L_{q}-Q
wherein:
L is absent or (1-4C)alkylene optionally substituted by one or more substituents selected from (1-2C)alkyl or oxo;
Y is absent or O, S, SO, SO₂, N(Rₐ), C(O), C(O)O, OC(O), C(O)N(Rₐ), N(Rₐ)C(O), C(O)N(Rₐ)—O—, N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, N(Rₐ)SO₂N(R_{b}) or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
L_{q} is absent or (1-4C)alkylene optionally substituted by one or more substituents selected from (1-2C)alkoxy, halo, cyano, amino or oxo; and
Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c}, R_{d} and Rₑ are each independently selected from hydrogen, (1-6C)alkyl, (3-6C)cycloalkyl or (3-6C)cycloalkyl(1-2C)alkyl; or
R_{c} and R_{d} are linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy; and/or
Q is optionally substituted by one or more group(s) of the formula:
-L₁-L_{Q1}-W₁
wherein:
L₁ is absent or (1-3C)alkylene optionally substituted by one or more substituents selected from (1-2C)alkyl or oxo;
L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
and wherein any alkyl, alkoxy, aryl, hereoaryl, heterocyclyl or cycloalkyl moiety in a substituent group present on W₁ is optionally further substituted by one or more halo, (1-4C)alkyl, (1-4C)haloalkyl, (1-4C)haloalkoxy, (1-4C)alkoxy, (1-4C)alkylamino, di-[(1-4C)alkyl]amino, amino, cyano or hydroxy groups; or
Rₕ and Rᵢ are linked such that, together with the nitrogen atom to which they are attached, they form a 4-7 membered heterocyclic ring which is optionally substituted by one or more substituents selected from oxo, (1-4C)alkyl, halo, (1-4C)haloalkyl, (1-4C)haloalkoxy, (1-4C)alkoxy, (1-4C)alkylamino, di-[(1-4C)alkyl]amino, amino, cyano or hydroxy;
A is selected from CR₄ and N,
wherein R₄ is hydrogen, halo or (1-4C)alkyl optionally substituted by one or more substituents selected from halo, (1-4C)haloalkyl, (1-4C)haloalkoxy, (1-4C)aminoalkyl, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and R_{4A} and R_{4B} are each independently selected from hydrogen, (1-6C)alkyl, (3-6C)cycloalkyl or (3-6C)cycloalkyl(1-2C)alkyl;
and wherein any tertiary amine in a compound of formula I is optionally in the form of a N-oxide and any nitrogen atom in a heteroaryl ring is optionally in the form of an N-oxide;
and wherein any S atoms present in the heterocyclic ring may optionally be present as S(═O), S(═O)₂ or S(═O)(═NR_{z}) wherein R_{z} is selected from hydrogen, (1-3C)alkyl or (2-3C)alkanoyl.

2. A compound according to claim 1, or a pharmaceutically acceptable salt thereof, wherein R₁ is selected from aryl or heteroaryl, wherein R₁ is optionally substituted by one or more R_{1z} substituents independently selected from (1-4C)alkyl, halo, (1-4C)haloalkyl, (1-4C)haloalkoxy, cyano, (CH₂)_{q}1NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B} (where p is 0, 1 or 2), SO₂N(R_{1C})R_{1B}, or N(R_{1C})SO₂R_{1B} and wherein:
q1 is 0, 1 or 2; and
R_{1B} and R_{1C} are each independently selected from hydrogen, (1-4C)alkyl, (3-6C)cycloalkyl or (3-6C)cycloalkyl(1-2C)alkyl.

3. A compound according to any one of the preceding claims, or a pharmaceutically acceptable salt thereof, wherein R₁ is selected from aryl or heteroaryl,
wherein R₁ is optionally substituted by one or more R_{1z} substituents independently selected from (1-2C)alkyl, halo, (1-2C)haloalkyl, (1-2C)haloalkoxy, cyano, (CH₂)_{q1}NR_{1B}R_{1C}, OR_{1B}, C(O)R_{1B}, C(O)OR_{1B}, OC(O)R_{1B}, C(O)N(R_{1C})R_{1B}, N(R_{1C})C(O)R_{1B}, S(O)ₚR_{1B}(where p is 0, 1 or 2), SO₂N(R_{1C})R_{1B}, or N(R_{1C})SO₂R_{1B} and wherein:
q1 is 0, 1 or 2; and
R_{1B} and R_{1C} are each independently selected from hydrogen, (1-2C)alkyl or (3-4C)cycloalkyl.

4. A compound according to any one of the preceding claims, or a pharmaceutically acceptable salt thereof, wherein R₁ is selected from phenyl, furyl, pyridyl or oxazolyl, wherein a phenyl, furyl, pyridyl or oxazolyl ring is optionally substituted by one or more of halo, (1-2C)alkyl, (1-2C)alkoxy or cyano.

5. A compound according to any one of the preceding claims, or a pharmaceutically acceptable salt thereof, wherein R₁ is selected from phenyl, furyl, pyridyl, or oxazolyl, wherein a phenyl, furyl, pyridyl or oxazolyl ring is optionally substituted by halo or cyano.

6. A compound according to any one of the preceding claims, or a pharmaceutically acceptable salt thereof, wherein R₁ is 3-cyanophenyl.

7. A compound according to any one of the preceding claims, or a pharmaceutically acceptable salt thereof, wherein R₂ is selected from hydrogen, cyano, halo, (1-4C)alkyl, (1-4C)haloalkyl, C(O)OR_{2A}, C(O)NR_{2A}R_{2B}, aryl, heteroaryl, heterocyclyl, (2-6C)alkenyl, (2-6C)alkynyl or (1-4C)alkanoyl;
wherein R_{2A} and R_{2B} are each independently selected from hydrogen, (1-4C)alkyl, (1-4C)alkoxy, (3-6C)cycloalkyl or (3-6C)cycloalkyl(1-2C)alkyl,
or, in the CONR_{2A}R_{2B} group, R_{2A} and R_{2B} are linked such that, together with the nitrogen atom to which they are attached, they form a 4-7 membered heterocyclic ring, and
wherein any alkyl, alkenyl, alkynyl, alkanoyl, aryl, heteroaryl or heterocyclyl group is optionally substituted by one or more substituents independently selected from (1-4C)alkyl, halo, (1-2C)haloalkyl, (1-2C)haloalkoxy, cyano, oxo, (CH₂)_{q2}NR_{2D}R_{2E}, (CH₂)_{q2}OR_{2D}, (CH₂)_{q2}C(O)R_{2D}, (CH₂)_{q2}C(O)OR_{2D}, (CH₂)_{q2}OC(O)R_{2D}, (CH₂)_{q2}C(O)N(R_{2E})R_{2D}, (CH₂)_{q2}N(R_{2E})C(O)R_{12D}, (CH₂)_{q2}S(O)ₚR_{2D} (where p is 0, 1 or 2), (CH₂)_{q2}SO₂N(R_{2E})R_{2D}, or (CH₂)_{q2}N(R_{2E})SO₂R_{2D},
wherein q2 is 0, 1, or 2; and
wherein R_{2D} and R_{2E} are each independently selected from hydrogen, (1-2C)alkyl, (3-4C)cycloalkyl or (3-4C)cycloalkyl(1-2C)alkyl.

8. A compound according to any one of the preceding claims, or a pharmaceutically acceptable salt thereof, wherein R₂ is selected from cyano, halo, methyl, CF₃, C(O)OR_{2A}, C(O)NR_{2A}R_{2B}, a 5 or 6-membered heteroaryl, a bicyclic heteroaryl or (2-4C)alkanoyl,
wherein R_{2A} and R_{2B} are each independently selected from hydrogen or (1-4C)alkyl,
wherein any heteroaryl group is optionally substituted by one or more substituents independently selected from (1-2C)alkyl, halo, (1-2C)haloalkyl, (1-2C)haloalkoxy, cyano, (CH₂)_{q2}NR_{2D}R_{2E}, (CH₂)_{q2}OR_{2D}, OR_{2D}, C(O)R_{2D}, C(O)OR_{2D}, OC(O)R_{2D}, C(O)N(R_{2E})R_{2D}, N(R_{2E})C(O)R_{12D}, S(O)ₚR_{2D} (where p is 0, 1 or 2), SO₂N(R_{2E})R_{2D}, or N(R_{2E})SO₂R_{2D}, wherein q2 is 0 or 1; and wherein R_{2D} and R_{2E} are each independently selected from hydrogen or (1-2C)alkyl.

9. A compound according to any one of the preceding claims, or a pharmaceutically acceptable salt thereof, wherein R₂ is selected from halo, a 5 or 6-membered heteroaryl, a bicyclic heteroaryl, wherein the 5 or 6-membered heteroaryl or bicyclic heteroaryl is optionally substituted as defined above in any one of claims 7 or 8.

10. A compound according to any one of the preceding claims, or a pharmaceutically acceptable salt thereof, wherein R₂ is either: 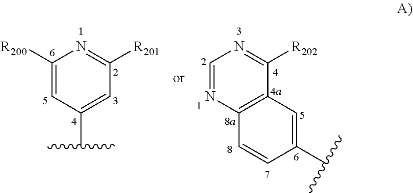
wherein:
(i) R₂₀₀ and R₂₀₁ are each independently selected from (1-2C)alkyl, halo, (1-2C)haloalkyl, (1-2C)alkoxy, (1-2C)haloalkoxy, (1-2C)hydroxyalkyl, (1-2C)alkanoyl or cyano; and
R₂₀₂ is selected from (1-2C)alkyl, halo, (1-2C)haloalkyl, (1-2C)alkoxy, (1-2C)haloalkoxy, (1-2C)hydroxyalkyl, (1-2C)alkanoyl or cyano;
(ii) R₂₀₀ and R₂₀₁ are each independently selected from methyl (including CD₃), halo, di-fluoromethyl, trifluoromethyl, methoxy, hydroxymethyl, acetyl or cyano; and
R₂₀₂ is selected from methyl (including CD₃), halo, di-fluoromethyl, trifluoromethyl, methoxy, hydroxymethyl, acetyl or cyano;
(iii) R₂₀₀ is methyl (including CD₃) or chloro and R₂₀₁ is selected from methyl (including CD₃), halo, di-fluoromethyl, trifluoromethyl, methoxy, hydroxymethyl, acetyl or cyano; and
R₂₀₂ is methyl or chloro
or 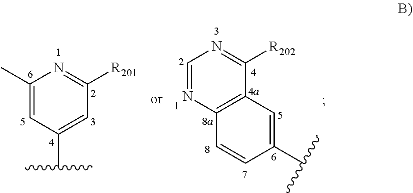
wherein:
(i) R₂₀₁ is (1-2C)alkyl, halo, (1-2C)haloalkyl, (1-2C)alkoxy, (1-2C)haloalkoxy, (1-2C)alkanoyl or cyano; and
R₂₀₂ is (1-2C)alkyl, halo, (1-2C)haloalkyl, (1-2C)alkoxy, (1-2C)haloalkoxy, (1-2C)alkanoyl or cyano;
(ii) R₂₀₁ is methyl (including CD₃), halo, di-fluoromethyl, trifluoromethyl, methoxy, acetyl or cyano;
R₂₀₂ is methyl (including CD₃), halo, di-fluoromethyl, trifluoromethyl, methoxy, acetyl or cyano;
(iii) R₂₀₁ is methyl (including CD₃) or chloro;
R₂₀₂ is methyl (including CD₃) or chloro.

11. A compound according to any one of the preceding claims, or a pharmaceutically acceptable salt thereof, wherein R₂ is:
bromo;
2-acetyl-6-methylpyridin-4-yl;
2,6-dimethylpyridin-4-yl;
2-Chloro-6-methylpyridin-4-yl
2-methyl-6-(trifluoromethyl)pyridine-4-yl;
2-methoxy-6-methyl-4-pyridyl;
2-(difluoromethyl)-6-methyl-4-pyridyl;
2-chloro-6-methyl-4-pyridyl;
2-chloro-6-methylpyridin-4-yl or 2,6-dimethylpyridin-4-yl;
2,6-bis(trideuteriomethyl)pyridyl;
4-methylquinazolin-6-yl;
2-(hydroxymethyl)-6-methyl-4-pyridyl.

12. A compound according to any one of the preceding claims, or a pharmaceutically acceptable salt thereof, wherein R₃ is selected from hydrogen, halo, cyano or a group of the formula:
-L-Y-L_{q}-Q
wherein:
L is absent or (1-4C)alkylene;
Y is absent or O, S, SO, SO₂, N(Rₐ), C(O), C(O)O, OC(O), C(O)N(Rₐ), N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
L_{q} is absent or (1-4C)alkylene optionally substituted by one or more substituents selected from (1-2C)alkoxy, halo, cyano, amino or oxo; and
Q is hydrogen, (1-6C)alkyl, (2-6C)alkenyl, (2-6C)alkynyl, aryl, (3-8)cycloalkyl, (3-8C)cycloalkenyl, heteroaryl or heterocyclyl;
wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, OC(O)R_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen, (1-6C)alkyl, (3-6C)cycloalkyl or (3-6C)cycloalkyl(1-2C)alkyl; and/or
Q is optionally substituted by one or more group(s) of the formula:
-L₁-L_{Q1}-W₁
wherein:
L₁ is absent or (1-3C)alkylene;
L_{Q1} is absent or selected from or O, S, SO, SO₂, N(R_{f}), C(O), C(O)O, OC(O), C(O)N(R_{f}), N(R_{f})C(O), N(R_{f})C(O)N(R_{g}), N(R_{f})C(O)O, OC(O)N(R_{f}), S(O)₂N(R_{f}), N(R_{f})SO₂ wherein R_{f} and R_{g} are each independently selected from hydrogen or (1-2C)alkyl; and
W₁ is hydrogen, (1-6C)alkyl, aryl, aryl(1-2C)alkyl, (3-8C)cycloalkyl, (3-8C)cycloalkenyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, aryl, heteroaryl, heterocycyl, (3-6C)cycloalkyl, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), SO₂N(Rᵢ)Rₕ, N(Rᵢ)SO₂Rₕ or (CH₂)ₛNRᵢRₕ (where s is 1, 2 or 3); wherein Rₕ and Rᵢ are each independently selected from hydrogen, (1-4C)alkyl, (3-6C)cycloalkyl or (3-6C)cycloalkyl(1-2C)alkyl;
and wherein any tertiary amine in a R₃ group is optionally in the form of a N-oxide.

13. A compound according to any one of the preceding claims, or a pharmaceutically acceptable salt thereof, wherein R₃ is selected from hydrogen, halo, cyano or a group of the formula:
-L-Y-L_{q}-Q
wherein:
L is absent or (1-2C)alkylene;
Y is absent or O, N(Rₐ), C(O), C(O)O, C(O)N(Rₐ), N(Rₐ)C(O), N(Rₐ)C(O)N(R_{b}), N(Rₐ)C(O)O, OC(O)N(Rₐ), C(═NR_{y})N(Rₐ), N(Rₐ)C(═NR_{y}), N(Rₐ)C(═NR_{y})N(R_{b}), S(O)₂N(Rₐ), N(Rₐ)SO₂, or C(O)N(Rₐ)SO₂, wherein Rₐ and R_{b} are each independently selected from hydrogen or (1-4C)alkyl and R_{y} is selected from hydrogen, (1-4C)alkyl, nitro or cyano;
L_{q} is absent or (1-4C)alkylene optionally substituted by one or more substituents selected from (1-2C)alkoxy, halo, cyano, amino or oxo; and
Q is hydrogen, (1-6C)alkyl, aryl, (3-8)cycloalkyl, heteroaryl or heterocyclyl;
wherein Q is optionally further substituted by one or more substituent groups independently selected from oxo, (1-4C)alkyl, halo, (1-4C)haloalkyl, (1-4C)haloalkoxy, (1-4C)aminoalkyl, (1-4C)hydroxyalkyl, cyano, NR_{c}R_{d}, OR_{c}, C(O)R_{c}, C(O)OR_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen or (1-6C)alkyl; and/or
Q is optionally substituted by one or more group(s) of the formula:
-L₁-L_{Q1}-W₁
wherein:
L₁ is absent or (1-2C)alkylene;
L_{Q1} is absent; and
W₁ is hydrogen, (1-6C)alkyl, aryl, aryl(1-2C)alkyl, (3-8C)cycloalkyl, heteroaryl or heterocyclyl; wherein W₁ is optionally substituted by one or more substituents selected from oxo, (1-4C)alkyl, halo, (1-4C)haloalkyl, (1-4C)haloalkoxy, (1-4C)alkoxy, (1-4C)alkylamino, cyano, NRₕRᵢ, ORₕ, C(O)Rₕ, C(O)ORₕ, OC(O)Rₕ, C(O)N(Rᵢ)Rₕ, N(Rᵢ)C(O)Rₕ, S(O)ᵣRₕ (where r is 0, 1 or 2), wherein Rₕ and Rᵢ are each independently selected from hydrogen or (1-4C)alkyl;
and wherein any tertiary amine in a R₃ group is optionally in the form of a N-oxide.

14. A compound according to any one of the preceding claims, or a pharmaceutically acceptable salt thereof, wherein R₃ is a group of the formula:
-L-Y-L_{q}-Q
wherein:
L is absent;
Y is N(Rₐ) or C(O)N(Rₐ);
L_{q} is absent; and
Q is (1-6C)alkyl or (3-8C)cycloalkyl;
wherein Q is optionally further substituted by one or more substituent groups independently selected from halo, cyano, NR_{c}R_{d}, OR_{c}, C(O)R_{c}, C(O)OR_{c}, C(O)N(R_{d})R_{c}, N(R_{d})C(O)R_{c}, S(O)ₚR_{c} (where p is 0, 1 or 2), SO₂N(R_{d})R_{c}, N(R_{d})SO₂R_{c}, or (CH₂)_{q}NR_{c}R_{d} (where q is 1, 2 or 3); wherein R_{c} and R_{d} are each independently selected from hydrogen or (1-6C)alkyl

15. A compound according to any one of the preceding claims, or a pharmaceutically acceptable salt thereof, wherein A is selected from CR₄ and N,
wherein R₄ is hydrogen, halo or (1-2C)alkyl optionally substituted by one or more substituents selected from halo, (1-2C)haloalkyl, (1-2C)haloalkoxy, amino, cyano, (CH₂)_{qa}NR_{4A}R_{4B}, (CH₂)_{qa}OR_{4A}, (CH₂)_{qa}C(O)R_{c4A}, (CH₂)_{qa}C(O)OR_{4A}, (CH₂)_{qa}OC(O)R_{4A}, (CH₂)_{qa}C(O)N(R_{4B})R_{4A}, (CH₂)_{qa}N(R_{4B})C(O)R_{4A}, (CH₂)_{qa}S(O)ₚR_{4A} (where p is 0, 1 or 2), (CH₂)_{qa}SO₂N(R_{4B})R_{4A}, or (CH₂)_{qa}N(R_{4B})SO₂R_{4A}, wherein qa is 0, 1, 2 or 3 and wherein R_{4A} and R_{4B} are each independently selected from hydrogen, (1-4C)alkyl, (3-4C)cycloalkyl or (3-4C)cycloalkyl(1-2C)alkyl.

16. A compound according to any one of the preceding claims, or a pharmaceutically acceptable salt thereof, wherein A is selected from CR₄ and N, wherein R₄ is hydrogen, halo or (1-2C)alkyl optionally substituted by one or more substituents selected from halo.

17. A compound according to any one of the preceding claims, or a pharmaceutically acceptable salt thereof, wherein A is selected from CR₄ and N, wherein R₄ is hydrogen, methyl or halo.

18. A compound of the formula: 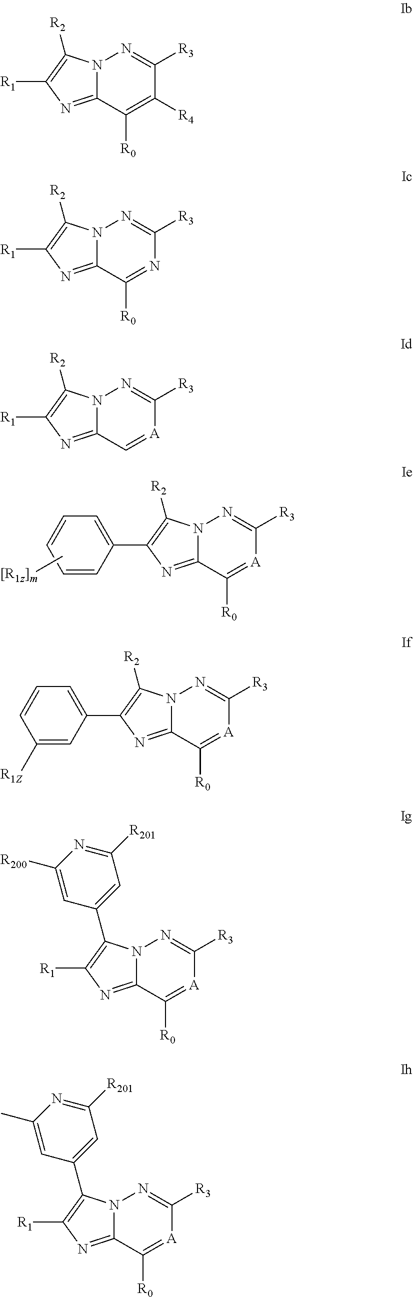 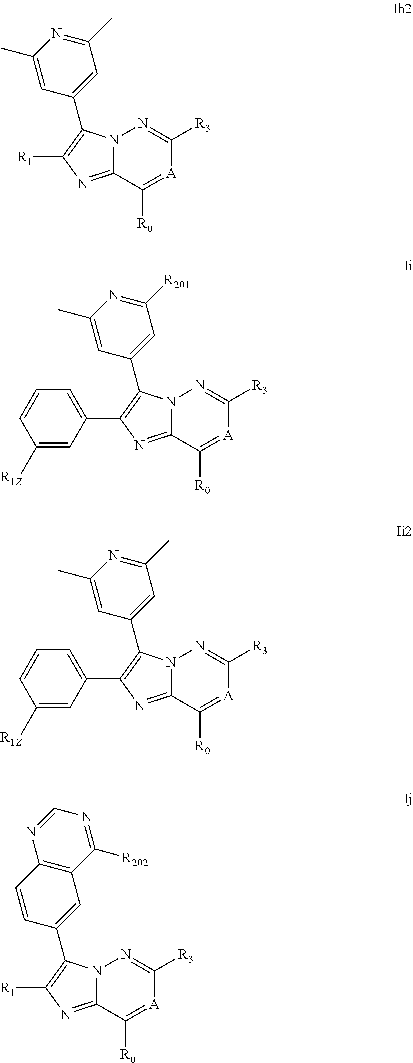
wherein A, R₀, R₁, R₂, R₃ and R_{1Z} are each as defined in any one of claims 1 to 15;
m is 0, 1 or 2;
R₂₀₀, R₂₀₁ and R₂₀₂ are each as defined in claim 10;
or a pharmaceutically acceptable salt thereof.

19. A compound, or a pharmaceutically acceptable salt thereof, selected from any one of the following:
3-Bromo-2-(3-cyanophenyl)-N-(2-hydroxy-2-methyl-propyl)imidazo[1,2-b]pyridazine-6-carboxamide;
3-(2-Acetyl-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-3-(2,6-dimethyl-4-pyridyl)-N-(2-hydroxy-2-methyl-propyl)imidazo[1,2-b]pyridazine-6-carboxamide;
3-(2-Chloro-6-methyl-4-pyridyl)-2-(3-cyanophenyl)-N-(2-hydroxy-2-methyl-propyl)imidazo[1,2-b]pyridazine-6-carboxamide;
2-(3-Cyanophenyl)-N-(2-hydroxy-2-methyl-propyl)-3-[2-methyl-6-(trifluoromethyl)-4-pyridyl]imidazo[1,2-b]pyridazine-6-carboxamide;
2-(3-Cyanophenyl)-N-(2-hydroxy-2-methyl-propyl)-3-(2-methoxy-6-methyl-4-pyridyl)imidazo[1,2-b]pyridazine-6-carboxamide;
2-(3-Cyanophenyl)-3-[2-(difluoromethyl)-6-methyl-4-pyridyl]-N-[(1S)-2-hydroxy-1,2-dimethyl-propyl]imidazo[1,2-b]pyridazine-6-carboxamide;
2-(3-Cyanophenyl)-N-[(1S)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1R)-2-hydroxy-1,2-dimethyl-propyl]-3-[2-methyl-6-(trifluoromethyl)-4-pyridyl]imidazo[1,2-b]pyridazine-6-carboxamide;
2-(3-Cyanophenyl)-N-[(1S)-2-hydroxy-1,2-dimethyl-propyl]-3-(2-methoxy-6-methyl-4-pyridyl)imidazo[1,2-b]pyridazine-6-carboxamide;
3-(2-Chloro-6-methyl-4-pyridyl)-2-(3-cyanophenyl)-N-[(1S)-2-hydroxy-1,2-dimethyl-propyl]imidazo[1,2-b]pyridazine-6-carboxamide;
2-(3-Cyanophenyl)-3-(2,6-dimethyl-4-pyridyl)-N-[(1S)-2-hydroxy-1,2-dimethyl-propyl]imidazo[1,2-b]pyridazine-6-carboxamide;
3-[2,6-Bis(trideuteriomethyl)-4-pyridyl]-2-(3-cyanophenyl)-N-[(1S)-2-hydroxy-1,2-dimethyl-propyl]imidazo[1,2-b]pyridazine-6-carboxamide;
2-(3-cyanophenyl)-N-[(1S)-2-hydroxy-1,2-dimethyl-propyl]-3-(4-methylquinazolin-6-yl)imidazo[1,2-b]pyridazine-6-carboxamide;
2-(3-cyanophenyl)-N-[(1S)-2-hydroxy-1,2-dimethyl-propyl]-3-[2-(hydroxymethyl)-6-methyl-4-pyridyl]imidazo[1,2-b]pyridazine-6-carboxamide.

20. A pharmaceutical composition comprising a compound according to any one of claims 1 to 19, or a pharmaceutically acceptable salt thereof, in admixture with a pharmaceutically acceptable diluent or carrier.

21. A compound according to any one of claims 1 to 19, or a pharmaceutically acceptable salt thereof, or a pharmaceutical composition according to claim 20, for use in therapy.

22. A compound according to any one of claims 1 to 19, or a pharmaceutically acceptable salt thereof, or a pharmaceutical composition according to claim 20, for use:
(i) in the treatment of a proliferative condition;
(ii) in the treatment of cancer;
(iii) in the treatment of cancer, wherein the compound or pharmaceutical composition is administered in combination with one or more additional anticancer agents;
(iv) in the treatment of cancer, wherein the compound or pharmaceutical composition is administered in combination with one or more additional anticancer agents selected from the group consisting of:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).

23. A method of treating a proliferative disorder in a patient in need of such treatment, the method comprising administering a therapeutically effective amount of a compound according to any one of claims 1 to 19, or a pharmaceutically acceptable salt thereof, or a pharmaceutical composition according to claim 20.

24. A method of treating cancer in a patient in need of such treatment, the method comprising administering a therapeutically effective amount of a compound according to any one of claims 1 to 19, or a pharmaceutically acceptable salt thereof, or a pharmaceutical composition according to claim 20.

25. A method of treating a proliferative disorder in a patient in need of such treatment, the method comprising administering a therapeutically effective amount of a compound according to any one of claims 1 to 19, or a pharmaceutically acceptable salt thereof, or a pharmaceutical composition according to claim 20 in combination with one or more additional anticancer agents.

26. A method according to claim 25, wherein the one or more additional anticancer agents is selected from:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).
